SEQUENCE LISTING (81 sequences)

SEQ ID NO: 1 - protein, 189 aa, Arabidopsis thaliana
  PEPTIDE 1..189
MVHSKKFRGVRQRQWGSWVSEIRHPLLKRRVWLGTFDTAETAARAYDQAAVLMNGQSAKTNFPVIKSNGSNSLEINSALRSPKSLSELLNAKLRKNCKDQTPYLTCLRLDNDSSHIGVWQKRAGSKTSPNWVKLVELGDKVNARPGGDIETNKMKVRNEDVQEDDQMAMQMIEELLNWTCPGSGSIAQV

SEQ ID NO: 2 - protein, 199 aa, Arabidopsis thaliana
MVQTKKFRGVRQRHWGSWVAEIRHPLLKRRIWLGTFETAEEAARAYDEAAVLMSGRNAKTNFPLNNNNTGETSEGKTDISASSTMSSSTSSSSLSSILSAKLRKCCKSPSPSLTCLRLDTASSHIGVWQKRAGSKSDSSWVMTVELGPASSSQETTSKASQDAILAPTTEVEIGGSREEVLDEEEKVALQMIEELLNTN

SEQ ID NO: 3 - protein, 189 aa, Arabidopsis thaliana
  PEPTIDE 1..189
MVHSRKFRGVRQRQWGSWVSEIRHPLLKRRVWLGTFETAEAAARAYDQAALLMNGQNAKTNFPVVKSEEGSDHVKDVNSPLMSPKSLSELLNAKLRKSCKDLTPSLTCLRLDTDSSHIGVWQKRAGSKTSPTWVMRLELGNVVNESAVDLGLTTMNKQNVEKEEEEEEAIISDEDQLAMEMIEELLNWS

SEQ ID NO: 4 - protein, 274 aa, Arabidopsis thaliana
  PEPTIDE 1..274
MGRSPCCEKDHTNKGAWTKEEDDKLISYIKAHGEGCWRSLPRSAGLQRCGKSCRLRWINYLRPDLKRGNFTLEEDDLIIKLHSLLGNKWSLIATRLPGRTDNEIKNYWNTHVKRKLLRKGIDPATHRPINETKTSQDSSDSSKTEDPLVKILSFGPQLEKIANFGDERIQKRVEYSVVEERCLDLNLELRISPPWQDKLHDERNLRFGRVKYRCSACRFGFGNGKECSCNNVKCQTEDSSSSSYSSTDISSSIGYDFLGLNNTRVLDFSTLEMK

SEQ ID NO: 5 - protein, 282 aa, Arabidopsis thaliana
  PEPTIDE 1..282
MGRSPCCEKAHTNKGAWTKEEDERLVAYIKAHGEGCWRSLPKAAGLLRCGKSCRLRWINYLRPDLKRGNFTEEEDELIIKLHSLLGNKWSLIAGRLPGRTDNEIKNYWNTHIRRKLINRGIDPTSHRPIQESSASQDSKPTQLEPVTSNTINISFTSAPKVETFHESISFPGKSEKISMLTFKEEKDECPVQEKFPDLNLELRISLPDDVDRLQGHGKSTTPRCFKCSLGMINGMECRCGRMRCDVVGGSSKGSDMSNGFDFLGLAKKETTSLLGFRSLEMK

SEQ ID NO: 6 - protein, 269 aa, Arabidopsis thaliana
  PEPTIDE 1..269
MGRSPCCEKEHMNKGAWTKEEDERLVSYIKSHGEGCWRSLPRAAGLLRCGKSCRLRWINYLRPDLKRGNFTHDEDELIIKLHSLLGNKWSLIAARLPGRTDNEIKNYWNTHIKRKLLSKGIDPATHRGINEAKISDLKKTKDQIVKDVSFVTKFEETDKSGDQKQNKYIRNGLVCKEERVVVEEKIGPDLNLELRISPPWQNQREISTCTASRFYMENDMECSSETVKCQTENSSSISYSSIDISSSNVGYDFLGLKTRILDFRSLEMK

SEQ ID NO: 7 - protein, 243 aa, Oryza sativa
  PEPTIDE 1..243
MGQSKKKFRGVRQRHWGSWVSEIRHPLLKRRVWLGTFETAEEAARAYDEAAILMSGRNAKTNFPVARNATGELTPAAAVAGRDGRVGGGSGSSSSMTANGGGNSLSQILSAKLRKCCKTPSPSLTCLRLDPEKSHIGVWQKRAGARADSSWVMTVELNKDTAVSSAATVAAATAVSSSDQPTPSDSTVTTTSTSTTGSPSPPPPAMDDEERIALQMIEELLGRSGPGSPSHGLLHGGEGSLVI

SEQ ID NO: 8 - protein, 243 aa, Oryza sativa; identical to SEQ ID NO: 7
  PEPTIDE 1..243

SEQ ID NO: 9 - protein, 243 aa, Oryza sativa
  PEPTIDE 1..243
MGRSPCCEKEHTNKGAWTKEEDERLVAYIRAHGEGCWRSLPKAAGLLRCGKSCRLRWINYLRPDLKRGNFTADEDDLIIKLHSLLGNKWSLIAARLPGRTDNEIKNYWNTHIRRKLLGRGIDPVTHRPVNAAAATISFHPQPPPTTKEEQLILSKPPKCPDLNLDLCISPPSCQEEDDDYEAKPAMIVRAPELQRRRGGLCFGCSLGLQKECKCSGGGAGAGAGNNFLGLRAGMLDFRSLPMK

SEQ ID NO: 10 - protein, 251 aa, Oryza sativa
  PEPTIDE 1..251
MGRSPCCEKAHTNKGAWTKEEDDRLIAYIKAHGEGCWRSLPKAAGLLRCGKSCRLRWINYLRPDLKRGNFTEEEDELIIKLHSLLGNKWSLIAGRLPGRTDNEIKNYWNTHIRRKLLSRGIDPVTHRPINDSASNITISFEAAAAAARDDKAAVFRREDHPHQPKAVTVAQEQQAAADWGHGKPLKCPDLNLDLCISLPSQEEPMMMKPVKRETGVCFSCSLGLPKSTDCKCSSFLGLRTAMLDFRSLEMK

SEQ ID NO: 11 - protein, 268 aa, Populus trichocarpa
  PEPTIDE 1..268
MGRSPCCEKAHTNKGAWTKEEDDRLIAYIRTHGEGCWRSLPKAAGLLRCGKSCRLRWINYLRPDLKRGNFTEEEDELIIKLHSLLGNKWSLIAGRLPGRTDNEIKNYWNTHIRRKLLNRGIDPATHRPLNEPAQEASTTISFSTTTSVKEESLSSVKEESNKEKIISAAAFICKEEKTPVQERCPDLNLELRISLPCQNQPDRHQAFKTGGSTSLCFACSLGLQNSKDCSCSVIVGTIGSSSSAGSKTGYDFLGMKSGVLDYRGLEMK

SEQ ID NO: 12 - protein, 271 aa, Populus trichocarpa
  PEPTIDE 1..271
MGRSPCCEKAHTNKGAWTKEEDDRLVAYIRAHGEGCWRSLPKAAGLLRCGKSCRLRWINYLRPDLKRGNFTEAEDELIIKLHSLLGNKWSLIAGRLPGRTDNEIKNYWNTHIRRKLLNRGIDPATHRPLNEPAVQEATTTISFTTTTTSVLEEESLGSIIKEENKEKIISATAFVCKEEKTQVQERCPDLNLELGISLPSQNQPDHHQPFKTGGSRSLCFACSLGLQNSKDCSCNVIVSTVGSSGSTSTKTGYDFLGMKSGVLDYRSLEMK

SEQ ID NO: 13 - protein, 105 aa, Sorghum bicolor
  PEPTIDE 1..105
MPTPTPTPTPTCGDGSLAGFALLLRGEKRVANGARGGRGIGGERAKIIRRRHAEKTHGRRERGGHRRSHRLAYPLWVLDIRSPNGIMLGIFRGAALWLWTLAWHM

SEQ ID NO: 14 - protein, 235 aa, Sorghum bicolor
  PEPTIDE 1..235
MVQSKKKFRGVRQRHWGSWVSEIRHPLLKRRVWLGTFETAEEAARAYDEAAVLMSGRNAKTNFPVPRTATGELAPVPAARDARGGGGSSSAAAAPGGGTSNLSQILSAKLRKCCKTPSPSLTCLRLDPEKSHIGVWQKRAGARADSSWVMTVQLNKDVPPPASSSGEEPVPSDGGAAATTPTSTSTSSTVTTTGSPPPAMMMDDEERIALQMIEELLGSSHSHGMFQGAAGSIVI

SEQ ID NO: 15 - protein, 258 aa, Sorghum bicolor
  PEPTIDE 1..258
MGRSPCCEKAHTNKGAWTKEEDDRLVAYIRAHGEGCWRSLPKAAGLMRCGKSCRLRWINYLRPDLKRGNFTADEDDLIIKLHSLLGNKWSLIAARLPGRTDNEIKNYWNTHIRRKLLGRGIDPVTHRPIADAGAGTVTTISFQPNKPNAAVAAQAPQHQPIKAVATAVVKVPRCPDLNLDLCISPPCQQKEDEELDLKPAVVVKREVLQAGHGGSLCFGCSLGIQKGAPGCSCSSSNSHHRFLGLRSGMLDFRGLEMK

SEQ ID NO: 16 - protein, 264 aa, Sorghum bicolor
  PEPTIDE 1..264
MGRSPCCEKAHTNKGAWTKEEDDRLVAYIKAHGEGCWRSLPKAAGLLRCGKSCRLRWINYLRPDLKRGNFTEEEDELIIKLHSLLGNKWSLIAGRLPGRTDNEIKNYWNTHIRRKLLSRGIDPVTHRPINEHTSNITISFEAAAAARDREENKGAVFRLEEHNKATAAAAAAIGRDHHQNHHPAGDWGQGKPLKCPDLNLDLCISPPAAPCQEEKAMVTMKPVKREAGLCFSCSLGLPKSADCKCSNFLGLRTAMLDFRSLEMK

SEQ ID NO: 17 - protein, 213 aa, Zea mays
  PEPTIDE 1..213
MTENLHSRKMVQPKKFRGVRQRHWGSWVSEIRHPLLKRRVWLGTFETAEEAARAYDEAAVLMSGRNAKTNFPIQRSSTGEPTPAAGRDARSNFSSGSSTTNLSQILSAKLRKCCKAPSPSLTCLRLDPEKSHIGVWQKRAGARADSNWVMTVELNKDAASTDAASQSTSATTAPPATPMDEEERIALQMIEELLSSSSPASPSNGDDQGRFII

SEQ ID NO: 18 - protein, 257 aa, Zea mays
  PEPTIDE 1..257
MGRSPCCEKAHTNRGAWTKEEDERLVAYVRAHGEGCWRSLPRAAGLLRCGKSCRLRWINYLRPDLKRGNFTADEDDLIVKLHSLLGNKWSLIAARLPGRTDNEIKNYWNTHIRRKLLGSGIDPVTHRRVAGGAATTISFQPSPNTAVAAAAETAAQAPIKAEETAAVKAPRCPDLNLDLCISPPCQHEDDGEEEEEELDLIKPAVVKREALQAGHGHGHGLCLGCGLGGQKGAAGCSCSNGHHFLGLRTSVLDFRGL

SEQ ID NO: 19 - protein, 273 aa, Zea mays
  PEPTIDE 1..273
MGRSPCCEKAHTNKGAWTKEEDERLVAHIRAHGEGCWRSLPKAAGLLRCGKSCRLRWINYLRPDLKRGNFTEEEDELIVKLHSVLGNKWSLIAGRLPGRTDNEIKNYWNTHIRRKLLSRGIDPVTHRPVTEHHASNITISFETEVAAAARDDKKGAVFRLEEEEERNKATMVVGRDRQSQSQSHSHPAGEWGQGKRPLKCPDLNLDLCISPPCQEEEEMEEAAMRVRPAVKREAGLCFGCSLGLPRTADCKCSSSSFLGLRTAMLDFRSLEMK

SEQ ID NO: 20 - protein, 256 aa, Panicum virgatum
  PEPTIDE 1..256
MGRSPCCEKAHTNKGAWTKEEDDRLVAYIRAHGEGCWRSLPKAAGLLRCGKSCRLRWINYLRPDLKRGNFTADEDDLIVKLHSLLGNKWSLIAARLPGRTDNEIKNYWNTHIKRKLLSRGIDPVTHRPIADAARNVTISFQPDAPSQQQLSDDAEAPPPPPPQQQQQLKPPPRCPDLNLDLCISPPCHKEEEDQELVKPAAVKREMLQAGHGTLGLCFGCSLGLQKGAAGCTCSSNSHFLGLRVGMLLDFRGLEMK

SEQ ID NO: 21 - DNA, 1014 bp, Arabidopsis thaliana
  regulatory 1..1000: note = tissue specific promoter; regulatory_class = promoter
  5'UTR 1001..1014
  source 1..1014: mol_type = genomic DNA
acgtacctcgtgtccaccggtgactctatccccggcgttagaagtgatgatagtctcgttcccaagggaaatcagccttcgaattggaattgatccctccggacattttgtgccgttcgtgtgccagacttgccatccatataatgcatcttcttctttttttcccgcagatggcatgtccgttggtctttcctgtatcatttatttacaaaagaaaaataaattaaacatttattaagttccccccgtaaaaaaaaaatatatatatatatatatatataacacatgcatcataattggtatgtccgtaggtgtttccttatcataactgaaccattggtaaactatcggttccgttaaagcataagactagaaaaggctcggtgcgactcgctaccacgtttctaaagattttatttagcaaattaaccccaatatatattttgctatgagggtctaaacaaactggtatatgagccatttacttaccacttattagttccaagtatttattttttgggttaattaatgtttaaattattggttgacaaaaaatataaaaataatggttaagttattgaaatgacttgagcaatctgatgcaactgcggataacatgaactcattcgaagtgacgtcccaaatatttgattctttgtttttattcctttttgtcaaggtcaagattggccaaacattttcaatatctaaatatattgacattcatagcctggaaaagaaaaaatatatggttaaattagttccaaagtattctagcagcaacaaaaccgctccaataaacgatttccaatttctatctcaaacttgtttcccaatcattagttataatccgtcccctaaaccaaaaaaaaatctaattgtaaaggtgttgcaatagattaaccatttttattttattttggtaaaatagattaaccattttgttagaaaaatgaagtttaaaacatttacagttctacgtgtacatgcttcgaccaatatgcttcgaccaat

SEQ ID NO: 22 - DNA, 1296 bp, Arabidopsis thaliana
  regulatory 1..1000: note = tissue specific promoter; regulatory_class = promoter
  5'UTR 1000..1296
  source 1..1296: mol_type = genomic DNA
atacatgtcatgattttataattatgtatatataaatactaattgatgtatgaagtacgtagataatgttacgatctattaatctatttacattaacttttaattagtgttgagtagggaaaattaacatataaacctttagcagttggttgtattattaaaaataatttgaacttaaaatccaccttcgaaaagataaatcaaacaagtataaaaaatgctataaatccagaatatttacctaaggtttttattcttctacttaataatgtaagataaaaccggcacaatacttgttacgtatgcatggtaggtaccgcaattgtgtaagcaaatcggcacaatactaaggttacatatactaactaaataaaacaatctgatttcagtgacaccgtatatctaacctttattcaaatccaagggaacatgacttgacttcttctgttggaactaactcgatccctcaaccatctccagggatagaagagttagtaaaatcaaacttgaagtgaggaagtaagcagtttaacgactccatatgactacagttatatacaaagttgggcacaaagtacaagtactaaatactcaaagtcagataataattttaataagtacaaactatatatatgcagtacaattattgagtatatataaacgagactggtgatttggggcattgtccaccagggtgttatatcccaattgaaatttgaaaatttaagtgtgtgagtgttacgacaaaaaaaagtgtgtgaattgtaggcgcggtgaaaaggtaaattaagattggaactagaaaaatagttgaatatcctttactaaaagttgtcaattccggttttagtaaaaaaaaattttaaaatagaaattttatccaaaagacttcaaacacacatattcgcatatataacataagatatcattttttgtaaacagttaaaaagaaaaacacatgtttttttttttaatttagaaaaaaacatgttattatacaaaacagagttttgcccacttttaatatgttatgaaaagaaaaatgattttcttgggtttggtcagagagattggttgtggtaagaatgggaatcttaattacaaagaattggattttgggtcgacctaccacctaaaacgacgtcgcctccatctctggtttccaaatctctttctcctctccctttataagcttgcgttggccagtcgctcatctcgaaaacagagagaaaaagactaaaaacacagtttaagaagaaggagagatagagagagaagagaaagatagagagggag

SEQ ID NO: 23 - DNA, 1000 bp, Arabidopsis thaliana
  regulatory 1..1000: note = tissue specific promoter; regulatory_class = promoter
  source 1..1000: mol_type = genomic DNA
aactagaacacttcagataaattttgtcgttctgttgacttcatttattctctaaacacaaagaactatagaccataatcgaaataaaaaccctaaaaaccaaatttatctatttaaaacaaacattagctatttgagtttcttttaggtaagttatttaaggttttggagactttaagatgttttcagcatttatggttgtgtcattaatttgtttagtttagtaaagaaagaaaagatagtaattaaagagttggttgtgaaatcatatttaaaacattaataggtatttatgtctaatttggggacaaaatagtggaattctttatcatatctagctagttcttatcgagtttgaactcgggttatgattatgttacatgcattggtccatataaatctatgagcaatcaatataattcgagcattttggtataacataatgagccaagtataacaaaagtatcaaacctatgcaggggagaagatgatgaaaagaagagtgtgagccaatacaaagcagatttgaggacatggcttacaagtcttgggtacagagtttggggagtgatgggtgcacaatggaacagcttctctggttgtccagttcccaagagaaccttcaagctccctaactccatctactatgtcgcctgattaaatcttatttactaacaaaacaataagatcagagtttcattctgattcttgagtcttttttttctctctccctcttttcatttctggtttatataaccaattcaaatgcttatgatccatgcatgaaccatgatcatctttgtgtttttttttccttctgtattaccattttgggcctttgtgaaattgattttgggcttttgttatataatctcctctttctctttctctacctgattggattcaagaacatagccagatttggtaaagtttataagatacaaaatattaagtaagactaaagtagaaatacataataacttgaaagctactctaagtt

SEQ ID NO: 24 - DNA, 1167 bp, Arabidopsis thaliana
  regulatory 1..1000: note = tissue specific promoter; regulatory_class = promoter
  5'UTR 1000..1167
  source 1..1167: mol_type = genomic DNA
tacatcagtttcatcatctatcttgtttcttatagaagctcacaatcttcttcctggtcgagtttagaaatgtcagagagagttgtttccacagagacgtagaaacccataactttagtattcttcaacccttacaacttatctgagcaaaatcagaaggtcgaatttgatggatggttttgctgtatttggtcaacggttttatttgagacagtagaccagaggaaactcagatgtgatgatgcaaagactgaattggttaagagtgtagattgatttgttctaacattgcaaatgtagagtagaattatgcaaaaaacgttaatgaacagagaagtgattaagcagaaacaaaattagagaagtgatattatatctcaaaatttatttttggtacagctaaagctcaaattgttatagagattagagatattaaaccaaatgacgagtgttttctttagtagtaaacggtgaaaattctcttctgacaaagacaattaaaattttaggtttaagactttaatatttgtcacaaattgtcatttacctaaataaaaaaaaaactaaatattttttttagatacatatgtgtcttataattttaactataaattttaattttatgtcttaaataattgtttacactataaatttaaatattttaatgctaaaattaatttgattcaaaaaagtgattttaattcttatttttcttatagaaagttggtgattgaaaagatttacttaaaaattataacaacttcaatggtgaataacccgacccgaataaaccggatataacaacttcaatgttagcttgatatagaaagtacggtgacgcttaggaggcaagcaagctagtatctgccgctggttagagacaaagaacatgtgtcactcctctcaactaaaactttccttcactttcccgcaaaatcatttcaaaaaagctccaaatttagcttacccatcagctttctcagaaaaccagtgaaagaaacttctcaacttccgatttttcacaatccaccaaactttttttaataactttttttcctcttattacaaaacctccactctcatggcttctcaaacttgttatccatccaaatctcaatccctaattagggttcatttctctgtttctccaaacaggggaattcgaag

SEQ ID NO: 25 - DNA, 1019 bp, Arabidopsis thaliana
  regulatory 1..1000: note = tissue specific promoter; regulatory_class = promoter
  5'UTR 1000..1019
  source 1..1019: mol_type = genomic DNA
tcttcttgcatcaatgatatcaacaacaatgggtaataaagaagctacttcgaaattatatattttttcgtattctatattgatcatcagtcttaagtggtttggtttgttgcagtgaagaagaactatgtatggatctacgccaccgttcagttcggttttgtggtccttttcgctcagcttttctacagagttgtaagatttgatgtaatgtcacagagaaaccttactttgttgtcacagagaaaccttactttgttgaagagtttttgattcctcacactctctctcattaacttgtgtgtaggtgaagcagccggtaatgtgcattgtcttagccactatgatcggatttggactcaccatgaccggcacaacagctattaacgagtatttgaaatggaggagaagcaattcccacctgccagaagagccagcaagtactcaggtggtttgacagcagcgtagatcttttgagtgaagctagagtccctaaagggttggatcggttttcaattaaccggtcgggattcggttttcggtttagctttaatcgacttgtctaggttgagatcagatttggttttcaatacttccaagtctttttttttttgccaactaaaatataaggaatgatgataggcacacacatgacacataaaatcataatgaacagtagtatgattagcaatccatatttcttggataacacttcttcacagcttttttgacaggtcactataacacctttttcagttcatttttcattttcaatcctcacccacccaaactctcccttcaaagcaatgtctctcctctctctttctcaattcaaacaaactttattaaacctaaaagaaacatttccaatctctaatgacttagttgatagaatctcatttagttacctagtaataatcttcacactagtaagagaatcctactcttcaccaaactacatctctctctatataacaaaccccaaaacatctcaacatacacacacaacaactacaaca

SEQ ID NO: 26 - DNA, 1093 bp, Arabidopsis thaliana
  regulatory 1..1000: note = tissue specific promoter; regulatory_class = promoter
  5'UTR 1000..1093
  source 1..1093: mol_type = genomic DNA
tgcgaacagtttgattctgtttttctttttcctttttttgggtaattttcttataacttttttcatagtttcgattatttggataaaattttcagattgaggatcattttatttatttattagtgtagtctaatttagttgtataactataaaattgttgtttgtttccgaatcataagttttttttttttttggttttgtattgataggtgcaagagactcaaaattctggtttcgatgttaacagaattcaagtagctgcccacttgattcgatttgttttgtatttggaaacaaccatggctggtcaaggcccagcccgttgtgcttctgaacctgcctagtcccatggactagatctttatccgcagactccaaaagaaaaaggattggcgcagaggaattgtcatggaaacagaatgaacaagaaagggtgaagaagatcaaaggcatatatgatctttacattctctttagcttatgtatgcagaaaattcacctaattaaggacagggaacgtaacttggcttgcactcctctcaccaaaccttaccccctaactaattttaattcaaaattactagtattttggccgatcactttatataataagataccagatttattatatttacgaattatcagcatgcatatactgtatatagttttttttttgttaaagggtaaaataataggatccttttgaataaaatgaacatatataattagtataatgaaaacagaaggaaatgagattaggacagtaagtaaaatgagagagacctgcaaaggataaaaaagagaagcttaaggaaaccgcgacgatgaaagaaagacatgtcatcagctgatggatgtgagtgatgagtttgttgcagttgtgtagaaatttttactaaaacagttgtttttacaaaaaagaaataatataaaacgaaagcttagcttgaaggcaatggagactctacaacaaactatgtaccatacagagagagaaactaaaagcttttcacacataaaaaccaaacttattcgtctctcattgatcaccgttttgttctctcaagatcgctgctaatctccggccgtccct

SEQ ID NO: 27 - DNA, 1146 bp, Arabidopsis thaliana
  source 1..1146: mol_type = genomic DNA
tctctaattgtcaagtatcttagtctagagttaattacttaaatactaaaaggctgtcgacaaaatcaagcttgaatctccttgtggtatcttcaactcttcgttgtctgcttacgagtggtttactcagtaattatctataatatgttattttttttccctcatcttttagttgttgtttcattacattgaaaagcttgtaatgtctttatatggtatatatggatcttatgagtgaggcaagatccatgatgtttttgatcttagaatgtatatgatgatcttagaatgtatttgaccgcccacaaattattgttcattgggattatatctctagtccaactccaagcaatcgaaatgggtcctgcttttaagaacaacagtatatgtttaagaataataactttatatattctcgattttaagatcttttgacaaaacctccttttcgttaggagcgtactaatttccaagtgtttgattagtggggtctccgtaaatttatttagagtttctatctatttattaatagctcaattaattaatctatactgtatctaaacatcaatttatatatttactcttgagaccaaaactgtcaatttataacattggatagtttcttaattcttattatatatttttcaaacacttttcaagactaatctccacattaggtactctctctagagataaaaatatttatcaaaaacatttttatttatttattaagtagtagataaactactgtggcaaaatcgtaaatgtctaaatgctgatgaattttttttgctgctccaatctggtttagtgctccatatacatccacggccaaaatgaatctatggcggcattaagattcattagtaagcaacgattatattaatataattgtttttagcaatgattttccgtaatttcccaaatatgtttcagttaatgtgttccaatcccaacaactggttgttgcaaaagaccaccaacgcaagcaatcatcaaacatcaaaataatcttaccttagcgaacaaacaataactacacaattctcataaagctcttatatatcactaacttcacacattttgttttccacaaaaataaaaacggaactcactcaagaaaccttcttccttgaagagagggtt

SEQ ID NO: 28 - DNA, 1000 bp, Oryza sativa
  source 1..1000: mol_type = genomic DNA
tacagggtctcaagccaggatgacctcctttgaaacgtacgagtggtaaaacagtacgaagaacatcaaattttcatgagaattttcataggagacaggttaagagagaacttcaagagattggaccttatgttaacttcttctagagattggaccttatgttaactttcttctataaaatattagtgaagtgaggaaacttctaaaacaattatatggagtgatgaaaaaaattatttggtcagacggtaactatgagtactccataatccgtataagataataacatggtaattctattaggcgttcgccaacgaagccccaagcagccacccaaggtagctaggcggtgcctttgtccgtgtatcaaaaatctccatgcacgggagccattccaaataaaattttgaagctccaagtttttgttccgaaggatcaaatagaaaaattatccgtagaaattgaatcctaacaaaaattccccacaattcctctaattaaaacgaggccgaagcggcttcctgatcggacggctggaaggccatacatgtcctggcattaattatcactcaccttagattattacagctcggagctagaaagccctgcaagttgcaattaatggtgagtatgatctgatctgcagcgaaatgatctatcgatgtccctagttaagcagtcattgtgtcccttacccacctaaatccacgagtgtcagagctaagcgcgatcccgatccttaaaccccaaccccactctcttgctgctccatcaagcaaccaaccccaatccacacaccatacataattacatactaccagctaattaattactaataatgacttaatattccatcatctcccagctcagttatcacttcttgatcacaccccctaccattgattaacctcttccatctcactctacacgcctatataattagcctaatgatctcactttgcaaagcatctcattgcactaaactgctcactgcatttgc

SEQ ID NO: 29 - DNA, 1000 bp, Oryza sativa
  source 1..1000: mol_type = genomic DNA
actcgattccattagattattcacaaacccatgtgaaccgtgactgtcagtcaggtgaggaaacagatattccaaaaatcatattctttccttttgtattaaccaaattcacacaaaggtgatatttaaaactgacgaagatgaaaattaagttgacgcacgtaaaatgaaaaagctatcggcatatgattaattaaatttaattattacaaacttgataaatgaatatatttgatattttaaggcaacttctatataaaacttttttatatgaagaggactgctacaaaacgggatcccgttgcaacgggataaggcatattaactattctcttgcatgagtatcacagatatcaggccctaagtatcataggtaccaggtaacaggtatcacaggtatcgggtaccatacagctcgtaccacaacaggtatcaggtactatctcataaaaggtaacgtgataccgttgtctaggttttctgttatatgaaacatattgtagtttaaaaaacgtgccaacgaaaatagagataaaaatctgaatcttgatgagaaaatcatgcccaaatttcaccctaaaacagtcaatttcccgcgaaaaaaagcaaaaaaaaaaaaactccagacagttgttaaaaggggaaaaaaaaagacagaatgctcagccgtcgagacacacacaacggcaacgtcttaccagctcggagctctctcgcttgctgcctcttctcttcttcctcccgccaccgacaccacctccaccagcagcttcgccttcgccgcgtcggcatctgcagttgccacttcgctttcttccactccctcctcctcctcgcttacctcacactcctcccccccaatttcatcccccacccaccaccagatcccccaccacctgccgcattctcccccccaagatccagatcgccggtcacccccacgaactcgctcgagatccagagagagagagagaggcagtttcttggttgattttcgagg

SEQ ID NO: 30 - DNA, 1141 bp, Oryza sativa
  source 1..1141: mol_type = genomic DNA
gagttaaattgatatggttttgtctgtcaaggtgccgtttctatggttggaggaggaaaatgaaattaggggaatagaacagtcgcaatccaaaagctattgttctctcttctacggtagttgatagttcgatacgtgtgtttgatgtgagagactgagaggagtgcacggtgcacctgccccgtaaggacgcggaactacattatcagaggctaggccggtactgttatacgcgacgttcacgaatcacgatgcgtaaaaaagtgaagcatgaaacagtactaggctctccacgggccacatcatgaaaaaactggtgcgacgccacgcttaacaatttgtcggtcgtaaactcgtaaagttaaaagccccacgacattgtcaaccaatatctcagcacatgaacttctctaacgagtacaacgaaaccgcatccgcaaaagcgccgtgaaccaaagctcttgccgtgctgtgccgtgccggtggccgtgaacatgtggaacacgaagaactcttgcgcgagatcggagcacctgacctcccacctcgcgtccggcccgtcgccgtcgcagcaagcgggctgtcaaaaacgacgccacagcgcgagcgctctcgccgatccggcggacccaccctcctcacctcgcgcaccaaccgcccgttcgctagtccgatcccccacccctcatcccccctacgccttgcaggttacgcgcctcgccgcggccaacgcaaaccaaaccaaatcccccgtcaccttcgcttcgaaaccccgcaaaaccccatggaagaaaacaccgaacacctgccgcgcgcacgcctcctcctcccccgcctctcctcctctctcccgttccatgtccgctcaaccttgcttccattctttccatccacccgccgatcgacgcgatgccgacgccccaaccccacccaccgcctgccagcgccaccccacctcgcgcctctgcggctatggctatatcaccatgcctccaacctccggtacgcttagcctctctctctctccccctcccattctgcgcattgctctctgcgcgcggtcgcgtgctgctgctcgcggcgccccggagcgtctcctttgggggagaggagaggagaggagaggagaggggggtgagcc

SEQ ID NO: 31 - DNA, 1000 bp, Oryza sativa
  source 1..1000: mol_type = genomic DNA
ttcaatgcaggatgacgccaagagggagaaaccaagcagaggtggacggtacaacgtgtaagtcaccgcaaaacgttgcagcttggatagtggccatcgagtggtgtgccgataccggcgcctgttctttacagcctcagctagtgttgttgtccgaggcaatttttccgacctattgtgttgctttcctctctgatagcttatggtaaaagatacaaagatgttgaggagtttgtacgccacttaattttgctcgtaacatacattgacaatcaagaggagccatggcattgcgatctgcttacacggcatattcttactggatggtgtacactacttaccctttttaatgcaagcatcaatccattgcttttctcactgcacacctgattcgtactgaaaacgtgaaacataaaaaaaaacaaaaatctagctgatgttggctctcggggcctcgagtctagtttgtcctagatggctaacctgatatgtgttggtcacgctcacgtttgaaccgagaaagagtgtgtgtgtgtgtgtgtcggcgtgctgctacaccagagcctccctgaatcgcaatgcgtgttaacgccagcatcgcaggatttcatctcacttgacaggttcagatggccttcctcctaccgtctgccatttatacacgcagtgacttaacgcttacacgagccggatggcccggatctcccccctgcaccatctcaccagaaaaacggtgaggcgtcaccgcaacccacccaccaaacacatccacgtcccttcaccgttggccttcgattttgcttcagctgcactacgacccctccaacacatttccctcgcgtctcgttgcgatctcaccttacgacgatctcgttccagcagcagcagcatcggcagcggcggcttgcttccgaagcgagcaatgcatggcgcgcgcggccgcgtgcgtgcgtgccttggcttgcgctctaatcaaaccgggacgccccaactcacggtt

SEQ ID NO: 32 - DNA, 1000 bp, Oryza sativa
  source 1..1000: mol_type = genomic DNA
acaacccctgttgcaccaaacttgcttttttaagttttaactgaaattaggatagcaaagagagtactttaggcttcatgctacgagctgcctacgaccatagacaggcttaatcttgtcttatagttgtatcttgttgatcattaggtgcctaactgcctacataggcataatgcatcctttcatctgatctttgtcactgaccccatgtacagagtcctataagttgcaatgttctaatccttgtttcgtcagtcatttcgtacacatggatgaaagtctggggtttaaccaccgatgcgatccaatcttctcctacagtcgatgataaggaaatcgtaacaaagaatgtgaatttttttgacgctacaaagaatgtgaatgatctgaccagtctatctttcaccgaactgaagcatatactctgaatgtctaagatcatacttagactgaactatatactctgaatatctaaagatctcatacattcatacttacgctgcaggttgcaaattctagtcattattacactcgagacctaaattatgattagtggggtgtactccgatagaacagtttacagttcagaactcaaaagctacgaatgaattcatgacaaaaggcgacaagtgatacgtattcgagaataaatgtgtgaacaaaggccgtctcaaaaaaaaaaaagaaaaaaaaaaagagaatccctttgcctgcactctaaaacccagcccgacccaactctttgtacatgaccagcaaaagcaccgtctgctgcgactttttttctcttgtgcaatctattgtcgggaaaaaagagaggagaattatcatatcatcacctaataaattgcaaccaccagaggtactgtcctctctatataactctttctcgggcattttgctggcacttgcctgttctagtatctatagctagctaactgttactgtacctcctcccatatatcatcttcatatttttgcagatcgataagcgagaaaa

SEQ ID NO: 33 - DNA, 1247 bp, Oryza sativa
  source 1..1247: mol_type = genomic DNA
catactttaccttgttgtataactgcatgcataagaatctgagagccattgctcaattcttttcaacgaagatgtgaactgttggaaggcaatgtaaaacgggaagcgctgtatgaaagaatatgacgcacatcgtcttttgttttttaagaattgagtatatattcgttgtggggaacagcctgatgatgggccccgggaattaacgctcgagcaacgttggaccattctgacatcgcgtttcctgattagcacaatgtttcgttttatttggaaattgaattgaatgtttctactgttattaattgcagacagtacaccaaacgaccaaatctatctgcaaacaattaaccaagaccaactggagaatttacagatgaatcactgtgttacacctgtaaactgtggctcctttgagaattgagttacaacaagagtttggagatgaacttgtagttcatctatatcatcttaattaaacaataatatttattcaggaatgcagttcagagactgcttaacacacacacacacaaaaaaaaaacctaaacctgaggcttgtactggaacaaggtcattagcaaggtgtcctctagactcccggaccgacactacccttggaagtcaaacgcagctggcacaaacaaacggagcctcggtgacgccggtaaaccgcaccaatcattgttaaaccaaaaaacgtgaacaacaaaccaaaaagaaactaaaaaaccgctaaaaagacgcaaaagagagagaaaaaaaatgaaagaagaaaagaaacgacgcggactcgctgacgacccgcggcccggtccaacccaacccccaccgcctctctcgccgaccccgtccactccgccgaattcccccccaaacccaaacccaacgcgacctcacctcacccgcacgacgacggcacgacgcgacgcgttgcccgagctgacggcttgacgacgcctccgtccccgtccggcaccaacccatcccaacccaacgctccccttttccactgaccaattgatagcccaacctcacctctcctctcctcctcccccctcctctcctcctcgcctccgcaccagcagtttcgtgcaccgcacttcacccacctacctcccccaacctccccatcaaaaactagtagtagcagtatccacccatccacgcacgcgaccgagcgcgatcgattcgaggcggcggcggcggaggaggaggagggggagtagatccggcgggcggcagcg

SEQ ID NO: 34 - DNA, 1000 bp, Zea mays
  source 1..1000: mol_type = genomic DNA
tttgtgctgagatcggcaccagctttcatttaatacagcctcagcttacctgaggcaattttcgcacctgttatgatgttgttttgctctcagataggtttatgtagcacaagaaagatatgttggagacgttgacgattttgtatgcaactaaatttctatcttaatatgccccgattcaacagcacccagtcgagtcattgcgttctggagattcttgcagcgcatttccatgtttaagaccttattatgaaatgtctggcattcgtggatccactgagcttctttctgcgaatgtgccatatcgtggcattggccgaagcaaccaaacatttgttgcccttttgtgtcggtgttttataaagtacctcaatgacgatacagcctcagggcgcttcctgcttttgcacttattcggagttcaggcgagttaacgaagttcagacggttctgaagagaggccgtgttgtgttttgtcggcgtggtatcgcgcaagcacatgtgtctttggtaagatggtctggatggctgtcctaccacctgccatttatacacacactgacttcaccgtcacactggcacgacatgagctcgccatcctaccagaaacgctgagacgtcaccggcaaccacccctctcgctcgctctggcctctgctcctgatttgatttggacagaaaactgggcagggcagggcgcgctcagcacgtttgcttcggaaacactgcgagtgtgcgacacatttcccggcttgatctcgaagcgagccctgatgtgtttgtcatgcacctgcctgccttggcttgtgctctaatcaacgccggactccccaactcacggttggtgcgggacgccaccccgccaccttaccgcccgcctcggcgcctcaccagtcaccacacctcgcgcctgccatcagctatatcaccgtggccacttccgtgtcccttcacggatacctcacccccacagcccccggtcgatcgctcggcaatcggc

SEQ ID NO: 35 - DNA, 1000 bp, Zea mays
  source 1..1000: mol_type = genomic DNA
aggcggggccggaggagggcaccagagaggctgctcaggagagagaaataatagaatatgtggtatagagtaaacatgagtgcggatgattgtggtatagagtaaagaattttgctgactaggacagaatattctttttagggtagaaatttagagtactatgagtgcggatagcctaaggaccactttaaatttgacacaatattgaaatttgaatggttttaacatttgaaggctgaaaaccaaaatactttgtagctaagtgttggaaacccgactcggccaataagtcgacagaccgtaaaataaggtcaatctaaactttatgataaatattcttgtttgatagcaatagcattgcaggaccaggacccaagggaagagaagatgccaaatcccatcgaggctaaagcaaaaacgatccaatttatgagcaaacccacactgaagtttcaaaattgttttctgaaaaaaaagtaaccagcaagttaaaaaatgagatggcgggaaagccaagtctcggttggtcgaggggttggttggggcgcagcctgacaagtgacaacggcagcaggatagtagcatcaggcgcaagccagcgcaggcggcagcgcgaggatttcgcttcacttagcggcaacggagacgctgcacccaaccaacacgagctccccctcacccgctgcgacgcgcgcgtcccacgagcggaagccccccgcgccgacgcgagcgcgggggctcgaccgaccgacccaacgcctccatctccaccgcgcgcaccaaatcgcactcccgtccgccccgccgatcgaacagccaccgctcacctctcccacccgccaaaaacctccggcctcctctcatattcatatagctagcccctgccacaaggtagagcgtcgctcacacctgcgtcgccctgcctcgcaatcgcgaatctgtcgagcacctgaggggtcggaggccgagagctagcctagcacgccggcctccgcgcgcg

SEQ ID NO: 36 - DNA, 1000 bp, Zea mays
  source 1..1000: mol_type = genomic DNA
catgtatataagtattcaatacataaatcagttctggtaaagagtgaattaagttaatggacgtgctgaaaatggtttggcttagtcttgttgtgttttatggaaaaattgtgtaggtcacgattacttctcaatgcaattgagaaaagttttaattgcaagccatttatggttatttattaaaaaaacaaggagtaacacgtcattgttcaaggcgccaagctaccacatcactatgattcaaagaaccacttcagattttactcaagattggaaattgaatggttttgatatttaaggttactttttaccgaaaaatattggaaacgatcagactgaaatcgacttgatctagaaattatgatagaatctcttgtttcatagcgggtccggtgggaagacaaaatgtgtaatcccgtcgatatgtgctttctaaatgctaaaaacgatccaatatacgacgtgtgctttctagcctgtttgtttgtctttaatctgtactagtttctatgttttttttctcattgaattacagctacagtagtctaaacaacagcgggctttaattcgaagcgaacaacacctgctgagtaagcaaacccacgctgaatagtttcagaaatgttttctggatgaatagcaaaattgtagtagcaacaggatagacggcgggaaagccaagtctcggttggtccggccgtccggacgcagcctgacaagggcagcagcatagcaatagcatcaggcgcaagccagcgcaggcggctttcgcttcacttagcggcaacggggacgcagcgcccgcacccaaccaacacgagctcctctcctcacccgccgcgacacgcgcgcggctccaacgcctccatctccatcgcgcgcaccaaatcgcactccgtccgccccgtcgatcgaacagccaccgctcacctctctcacccgccaaaacctcctcccctggcctcctctcatactcatatagctgagcagcccctgccac

SEQ ID NO: 37 - DNA, 570 bp, Arabidopsis thaliana
  source 1..570: mol_type = genomic DNA
atggtacattcgaagaagttccgaggtgtccgccagcgtcagtggggttcttgggtttctgagattcgtcatcctctcttgaagagaagagtgtggctaggaacattcgacacggcggaaacagcggctagagcctacgaccaagccgcggttctaatgaacggccagagcgcgaagactaacttccccgtcatcaaatcgaacggttcaaattccttggagattaactctgcgttaaggtctcccaaatcattatcggaactattgaacgctaagctaaggaagaactgtaaagaccagacaccgtatctgacgtgtctccgcctcgacaacgacagctcacacatcggcgtctggcagaaacgcgccgggtcaaaaacgagtccaaactgggtcaagcttgttgaactaggtgacaaagttaacgcacgtcccggtggtgatattgagactaataagatgaaggtacgaaacgaagacgttcaggaagatgatcaaatggcgatgcagatgatcgaggagttgcttaactggacctgtcctggatctggatccattgcacaggtctaa

SEQ ID NO: 38 - DNA, 600 bp, Arabidopsis thaliana
  source 1..600: mol_type = genomic DNA
atggtacagacgaagaagttcagaggtgtcaggcaacgccattggggttcttgggtcgctgagattcgtcatcctctcttgaaacggaggatttggctagggacgttcgagaccgcagaggaggcagcaagagcatacgacgaggccgccgttttaatgagcggccgcaacgccaaaaccaactttcccctcaacaacaacaacaccggagaaacttccgagggcaaaaccgatatttcagcttcgtccacaatgtcatcctcaacatcatcttcatcgctctcttccatcctcagcgccaaactgaggaaatgctgcaagtctccttccccatccctcacctgcctccgtcttgacacagccagctcccatatcggcgtctggcagaaacgggccggttcaaagtctgactccagctgggtcatgacggtggagctaggtcccgcaagctcctcccaagagactactagtaaagcttcacaagacgctattcttgctccgaccactgaagttgaaattggtggcagcagagaagaagtattggatgaggaagaaaaggttgctttgcaaatgatagaggagcttctcaatacaaactaa

SEQ ID NO: 39 - DNA, 570 bp, Arabidopsis thaliana
  source 1..570: mol_type = genomic DNA
atggtacattcgaggaagttccgaggtgtccgccagcgacaatggggttcttgggtctctgagattcgccatcctctattgaagagaagagtgtggcttggaactttcgaaacggcagaagcggctgcaagagcatacgaccaagcggctcttctaatgaacggccaaaacgctaagaccaatttccctgtcgtaaaatcagaggaaggctccgatcacgttaaagatgttaactctccgttgatgtcaccaaagtcattatctgagcttttgaacgctaagctaaggaagagctgcaaagacctaacgccttctttgacgtgtctccgtcttgatactgacagttcccacattggagtttggcagaaacgggccgggtcgaaaacaagtccgacttgggtcatgcgcctcgaacttgggaacgtagtcaacgaaagtgcggttgacttagggttgactacgatgaacaaacaaaacgttgagaaagaagaagaagaagaagaagctattattagtgatgaggatcagttagctatggagatgatcgaggagttgctgaattggagttga

SEQ ID NO: 40 - DNA, 825 bp, Arabidopsis thaliana
  source 1..825: mol_type = genomic DNA
atgggaaggtctccttgctgtgagaaagaccacacaaacaaaggagcttggactaaggaagaagacgataagctcatctcttacatcaaagctcacggtgaaggttgttggcgttctcttcctagatccgccggtcttcaacgttgcggaaaaagctgtcgtctccgatggattaactatctccgacctgatctcaagaggggtaacttcaccctcgaagaagatgatctcatcatcaaactacatagccttctcggtaacaagtggtctcttattgcgacgagattaccaggaagaacagataacgagattaagaattactggaacacacatgttaagaggaagctattaagaaaagggattgatccggcgactcatcgacctatcaacgagaccaaaacttctcaagattcgtctgattctagtaaaacagaggaccctcttgtcaagattctctcttttggtcctcagctggagaaaatagcaaatttcggggacgagagaattcaaaagagagttgagtactcagttgttgaagaaagatgtctggacttgaatcttgagcttaggatcagtccaccatggcaagacaagctccatgatgagaggaacctaaggtttgggagagtgaagtataggtgcagtgcgtgccgttttggattcgggaacggcaaggagtgtagctgtaataatgtgaaatgtcaaacagaggacagtagtagcagcagttattcttcaaccgacattagtagtagcattggttatgacttcttgggtctaaacaacactagggttttggattttagcactttggaaatgaaatga

SEQ ID NO: 41 - DNA, 849 bp, Arabidopsis thaliana
  source 1..849: mol_type = genomic DNA
atgggaaggtcaccgtgctgtgagaaagctcacacaaacaaaggagcatggacgaaagaagaggacgagaggctcgtcgcctacattaaagctcatggagaaggctgctggagatctctccccaaagccgccggacttcttcgctgtggcaagagctgccgtctccggtggatcaactatctccggcctgaccttaagcgtggaaacttcaccgaggaagaagacgaactcatcatcaagctccatagccttcttggcaacaaatggtcgcttattgccgggagattaccgggaagaacagataacgagataaagaactattggaacacgcatatacgaagaaagcttataaacagagggattgatccaacgagtcatagaccaatccaagaatcatcagcttctcaagattctaaacctacacaactagaaccagttacgagtaataccattaatatctcattcacttctgctccaaaggtcgaaacgttccatgaaagtataagctttccgggaaaatcagagaaaatctcaatgcttacgttcaaagaagaaaaagatgagtgcccagttcaagaaaagttcccagatttgaatcttgagctcagaatcagtcttcctgatgatgttgatcgtcttcaagggcatggaaagtcaacaacgccacgttgtttcaagtgcagcttagggatgataaacggcatggagtgcagatgcggaagaatgagatgcgatgtagtcggaggtagcagcaaggggagtgacatgagcaatggatttgattttttagggttggcaaagaaagagaccacttctcttttgggctttcgaagcttggagatgaaataa

SEQ ID NO: 42 - DNA, 810 bp, Arabidopsis thaliana
  source 1..810: mol_type = genomic DNA
atgggaagatctccttgctgcgagaaagaacacatgaacaaaggtgcttggactaaagaagaagatgagagactagtctcttacatcaagtctcacggtgaaggttgttggcgatctcttcctagagccgctggtctccttcgctgcggtaaaagctgccgtcttcggtggattaactatctccgacctgatctcaaaagaggaaactttacacatgatgaagatgaacttatcatcaagcttcatagcctcctaggcaacaagtggtctttgattgcggcgagattacctggaagaacagataacgagatcaagaactactggaacacacatataaagaggaagcttttgagcaaagggattgatccagccactcatagagggatcaacgaggcaaaaatttctgatttgaagaaaacaaaggaccaaattgtaaaagatgtttcttttgtgacaaagtttgaggaaacagacaagtctggggaccagaagcaaaataagtatattcgaaatgggttagtttgcaaagaagagagagttgttgttgaagaaaaaataggcccagatttgaatcttgagcttaggatcagtccaccatggcaaaaccagagagaaatatctacttgcactgcgtcccgtttttacatggaaaacgacatggagtgtagtagtgaaactgtgaaatgtcaaacagagaatagtagcagcattagctattcttctattgatattagtagtagtaacgttggttatgacttcttgggtttgaagacaagaattttggattttcgaagcttggaaatgaaataa

SEQ ID NO: 43 - DNA, 618 bp, Oryza sativa
  source 1..618: mol_type = genomic DNA
atggtacagccaaagaagaagtttcgtggagtcaggcagcggcactggggctcctgggtctctgagatcagacaccccctccttaaaaggagggtgtggctgggcacctttgagacggccgaggaggctgcgcgagcctacgatgaggctgctgtgctgatgagtggccgcaacgccaagaccaacttccccgtgcagaggaactccaccggtgatctcgccacggccgcagaccaggacgcccgtagcaatggcggtagcaggaactcctccgcgggcaacctgtcacagattctcagtgctaagctccgcaagtgctgcaaggcgccatctccgtccttaacctgcctccgcctcgaccccgagaagtcccacattggcgtgtggcaaaagcgcgcaggggcccgtgctgactccaactgggtgatgacggtggagctcaacaaagaggtagaaccaactgaacctgcagctcagcccacatcaacagcaacagcttcgcaagtgacaatggatgatgaggaaaagattgcgctgcaaatgatcgaggagttgctgagcaggagcagtccagcttcaccctcacatggagagggagagggtagctttgtcatctga

SEQ ID NO: 44 - DNA, 732 bp, Oryza sativa
  source 1..732: mol_type = genomic DNA
atgggacagtcgaagaagaagttccgcggagtcaggcagcgccactggggctcctgggtctccgagatcaggcaccctctccttaagaggagggtgtggctgggtacctttgagacggcggaggaggcggcgcgggcgtacgacgaggccgccatcctgatgagcggccgcaacgccaagaccaacttcccagtcgcgaggaacgccacgggggagctcacaccggcggctgcggtggcagggcgggatggccgtgtcggcggcggcagcggcagctcgtcctcaatgacggccaacggcggcgggaacagcctgtctcagatcctcagcgccaagctccgcaagtgctgcaagacgccgtcgccgtcgctcacctgcctccgccttgacccggagaagtcccacattggcgtctggcagaagcgcgccggcgcacgcgctgactccagctgggtcatgaccgtcgagctcaacaaggacacggccgtgtcgtcggctgcgacggtggcagcagcaacagcagtgtcgtccagcgaccagccgactccgagtgacagcacagtcacaacgacgtccacgtccaccacgggctcgccgtcgccaccacctccggcaatggacgacgaggagaggatcgcgctgcagatgatcgaggagctgctgggcaggagcggcccgggctcgccgtcacatgggctgctgcacggtggtgaaggtagcctcgtcatctga

SEQ ID NO: 45 - DNA, 732 bp, Oryza sativa
  source 1..732: mol_type = genomic DNA
atggggaggtcgccgtgctgcgagaaggagcacactaacaagggcgcgtggaccaaggaggaggacgagcgcctcgtcgcctacatccgcgcccacggcgagggctgctggcgctcgctccccaaggccgccggcctcctccgctgcggcaagagctgccgcctccgctggatcaactacctccgccccgacctcaagcgcggcaacttcaccgccgacgaggacgacctcatcatcaagctccacagcctcctcggcaacaagtggtctctgatcgcggcgaggctgccggggaggacggacaacgagatcaagaactactggaacacgcacatccgccggaagcttctcggcagggggatcgaccccgtcacgcaccgccccgtcaacgccgccgccgccaccatctccttccatccccagccgccgccaacgacgaaggaggagcagctcatactcagcaagccgcccaagtgccccgacctcaacctggacctctgcatcagcccgccgtcgtgccaggaagaagacgatgactatgaggcgaagccggcgatgatcgtgagggcgccggagctgcagcgccgccgcggcggcctctgcttcggctgcagcctcggcctccagaaggagtgcaagtgcagcggcggcggcgccggcgccggcgccggcaacaacttcctcggcctcagggctggcatgctcgacttcagaagcctccccatgaaatga

SEQ ID NO: 46 - DNA, 756 bp, Oryza sativa
  source 1..756: mol_type = genomic DNA
atggggaggtcaccgtgctgcgagaaggcacacaccaacaagggagcatggaccaaggaggaagatgaccggctcattgcctacatcaaggcgcacggcgaaggttgctggcgatcgctgcccaaggccgccggcctcctccgctgtggcaagagctgccgcctccggtggatcaactacctccggcctgacctcaagcgcggcaacttcaccgaggaggaggatgagctgatcatcaagcttcacagccttttaggcaacaaatggtctctgatagccgggaggttgccaggaagaacggacaacgagatcaagaactactggaacacgcacatcaggaggaagctgctgagccgtggcatcgacccggtgacacaccggccgatcaacgacagcgcgtccaacatcaccatatcattcgaggcggccgcggcggcggcgagggacgacaaggccgccgtgttccggcgagaggaccatcctcatcagccgaaggcggtgacagtggcacaggagcagcaggcagccgccgattggggccatgggaagccactcaagtgccctgacctcaatctggacctctgcatcagcctcccttcccaagaagagcccatgatgatgaagccggtgaagagggagaccggcgtctgcttcagctgcagcctggggctccccaagagcacagactgcaagtgcagcagcttcctgggactcaggacagccatgctcgacttcagaagcttggaaatgaaatga

SEQ ID NO: 47 - DNA, 807 bp, Populus trichocarpa
  source 1..807: mol_type = genomic DNA
atgggaaggtctccttgctgtgaaaaagctcatacaaacaaaggcgcatggactaaggaagaagatgatcgccttattgcttacattagaacccacggtgaaggttgctggcgttcacttcctaaagctgctggccttctaagatgcggcaagagctgcagacttcgttggatcaactatttaagacctgaccttaaacgtggcaattttactgaagaagaagatgagctcattatcaaactccatagtctcctcggcaacaaatggtcacttatagccggaaggttaccagggagaacagataatgagataaagaattattggaacacacatataagaaggaagctcttgaatagaggcatagatcctgcgactcataggccactcaatgaaccagcccaagaagcttcaacaacaatatctttcagcactactacctcagttaaagaagagtcgttgagttctgttaaagaggaaagtaataaggagaagataattagcgcagctgcttttatatgcaaagaagagaaaaccccagttcaagaaaggtgtccagacttgaatcttgaacttagaattagccttccttgccaaaaccagcctgatcgtcaccaggcattcaaaactggaggaagtacaagtctttgttttgcttgcagcttggggctacaaaacagcaaggactgcagttgcagtgtcattgtgggtactattggaagcagcagtagtgctggctccaaaactggctatgacttcttagggatgaaaagtggtgtgttggattatagaggtttggagatgaaatga

SEQ ID NO: 48 - DNA, 816 bp, Populus trichocarpa
  source 1..816: mol_type = genomic DNA
atgggaaggtctccttgctgtgaaaaagcccatacaaacaagggtgcgtggaccaaggaggaagacgatcgccttgttgcttacattagagctcacggtgaaggttgctggcgctcacttcctaaagccgctggccttcttagatgtggcaagagttgcagacttcgttggatcaactatttaagacctgaccttaaacgtggcaatttcaccgaagcagaagatgagctcattatcaaactccatagcctccttggaaacaaatggtcactcatagctggaagattaccagggagaacagataatgagataaagaattattggaacacacatataagaaggaagcttttgaacagaggcatagatcccgcaactcataggccactcaacgaaccagcagtacaagaagccacaacaacaatatctttcaccacgactactacttcagtacttgaagaagagtctctgggttctataattaaagaggaaaataaagagaagataattagcgcaactgctttcgtatgcaaagaagagaaaacccaagttcaagaaaggtgtccagacttgaatctcgagcttggaattagccttccttcccaaaaccagcctgatcatcaccagccattcaaaactggaggaagtagaagtctttgttttgcttgcagtttggggctacaaaacagcaaggattgcagctgcaatgttattgtgagcactgttgggagcagtggcagcactagcacaaagactggttatgacttcttgggcatgaaaagtggtgttttggattatagaagtttagagatgaaataa

SEQ ID NO: 49 - DNA, 642 bp, Sorghum bicolor
  source 1..642: mol_type = genomic DNA
atgacagagaatctccactccaagaaaatggtacagccaaagaagtttcgtggagtccggcagcgccactggggttcctgggtctccgagatcaggcatcccctccttaagaggagggtctggctgggcaccttcgagaccgctgaggaggcagcgagagcatatgacgaggctgccgtgctgatgagcggccgcaacgccaagaccaacttcccggtccaaaggagcagcacaggggagccaaccccagctgcgggaagggacgctcacagcaacgccggcagcggctcctctaccgccaacctgtcccagattctcagtgcgaagctccgcaaatgctgcaaggcgccatcgccctccctgacctgtctccgccttgaccctgagaagtcccacattggtgtttggcagaagcgtgcaggagcccgtgctgactccaactgggtcatgaccgtggagctcaacaaaggtgcagcatccactgatgctgcatcacagtccacatcagcaacaactgctccaccagccaccccgatggatgacgaggagaggatcgccctgcaaatgatcgaagagttgctgagcagcagcagcccagcttcaccctcgcacggagatgaccaaggtcgcttcatcatctga

SEQ ID NO: 50 - DNA, 708 bp, Sorghum bicolor
  source 1..708: mol_type = genomic DNA
atggtgcaatcaaagaagaagttccgcggcgtcaggcagcgccactggggctcctgggtctccgagatcaggcacccgctgcttaagaggagggtgtggctgggcaccttcgagacggcagaggaggcggcgcgggcgtacgacgaggccgccgtcctcatgagcggccgcaacgccaagaccaacttccccgtcccaaggaccgccaccggggagctggcccccgtgccggccgcgcgggacgcacgtggcggcggcggctcgtcctccgcggcagcagcgcccggcggcggcaccagcaacctgtcgcagatcctcagcgccaagctccgcaagtgctgcaagacgccgtcgccgtcgctcacctgcctccgcctcgacccggagaagtcccacattggcgtctggcagaagcgcgcgggcgcgcgcgccgactccagctgggtcatgaccgtccagctcaacaaggacgtgccgccgccggcgtcctcctccggcgaggagccggtgcccagcgacggaggcgcagcggccaccacgcccacgtccacttccacgtcgtccacggtcacgacgaccggctcgcctccacctgcgatgatgatggacgacgaggagaggattgcgctgcagatgatcgaggagctgctgggcagctcgcactcacatgggatgttccagggtgcagcgggcagcatcgtcatctga

SEQ ID NO: 51 - DNA, 777 bp, Sorghum bicolor
  source 1..777: mol_type = genomic DNA
atggggcggtcgccgtgctgcgagaaggcgcacacgaacaagggcgcgtggaccaaggaggaggacgaccgcctggtggcgtacatccgcgcgcacggcgaagggtgctggcggtcgctgcccaaggcggccggactgatgcgctgcggcaagagctgccgcctccgctggatcaactacctccgccccgacctcaagcgcggcaacttcaccgccgacgaggacgacctcatcatcaagctgcacagcctcctcggcaacaagtggtcgctcatcgccgcgcggctcccggggcggacggacaacgagatcaagaactactggaacacgcacatccggcggaagctgcttggcaggggcatcgaccccgtcacgcaccgccccatcgccgacgccggcgccggcaccgtcaccaccatctcgttccagcccaacaaacccaacgccgccgtcgcagcgcaggcgccacaacatcagccgatcaaggcggtggcgacggccgtcgttaaggtgcccaggtgccccgacctcaacctcgatctctgcatcagcccgccgtgccaacagaaggaagacgaggagctggacctcaagcccgccgtcgtcgtcaagcgggaggtgctgcaggccggccatggcggcagcctctgcttcggctgcagcctgggcatccaaaaaggagcccccgggtgcagctgcagcagcagcaacagccaccaccgcttcttggggctccggtccggcatgctcgacttcagaggcctcgagatgaagtga

SEQ ID NO: 52 - DNA, 795 bp, Sorghum bicolor
  source 1..795: mol_type = genomic DNA
atggggaggtcgccgtgctgcgagaaggcgcacaccaacaagggcgcgtggaccaaggaggaggacgaccgcctcgtggcgtacatcaaggcgcacggcgagggttgctggcgctcgctgcccaaggccgccggcctcctgcgctgcggcaagagctgccgcctccggtggatcaactacctccgccccgacctcaagcgcggcaacttcacggaagaggaggacgagctcatcatcaagctccacagcctcctcggcaacaaatggtccctgatcgctggaaggctgccgggaaggacggacaacgagatcaagaactactggaacacgcacatccggaggaagctgctgagcagggggatcgacccggtgacacaccgccccatcaacgagcacacgtccaacataaccatctcgttcgaggcggcggcggccgcgcgtgaccgtgaggagaataagggcgccgtgttccggctggaggagcacaacaaggcgacggcggcggcggccgccgcgatcggccgcgatcatcatcagaaccaccaccccgccggcgactggggccaggggaagccgctcaagtgccccgacctcaacctggacctctgcatcagcccgccggcggcgccgtgccaggaggagaaggccatggtgacgatgaagcccgtgaagcgggaggccgggctctgcttcagctgcagcctgggcctccccaagagcgccgactgcaagtgcagcaacttcctcggactcaggaccgccatgctcgacttcagaagcctcgagatgaaatga

SEQ ID NO: 53 - DNA, 642 bp, Zea mays
  source 1..642: mol_type = genomic DNA
atgacagagaatctccactccaggaaaatggtacagccaaagaagtttcgtggagtccggcagcgccactggggctcctgggtctctgagatcaggcatcccctccttaagaggagggtctggctgggtacctttgagacggctgaggaggcagcgagagcatatgatgaggctgctgtgctgatgagcggacgcaacgccaagaccaacttcccaatccaaagaagcagcacaggggagcctaccccagctgcgggaagggacgcccgcagcaacttcagcagcggctcctctaccaccaacctgtcccagattctcagtgcgaagctccgcaaatgctgcaaggcgccatcaccgtccctgacctgtctccgccttgaccctgagaagtcccacattggtgtttggcagaagcgtgcaggagcccgtgctgactccaactgggtcatgacagtggagctcaacaaagatgcagcatccactgatgctgcatcacagtccacatcagcaacaactgctccaccagccacgccgatggatgaggaggagaggatcgcactgcaaatgatcgaagagttgctgagcagcagcagcccagcttcaccctcaaacggagatgaccaaggtcgcttcatcatctga

SEQ ID NO: 54 - DNA, 783 bp, Zea mays
  source 1..783: mol_type = genomic DNA
atggggcggtcgccgtgctgcgagaaggcgcacaccaacaggggcgcgtggaccaaggaggaggacgagcggctggtggcctacgtccgcgcgcacggcgaagggtgctggcgctcgctgcccagggcggcgggcctgctgcgctgcggcaagagctgccgcctgcgctggatcaactacctccgcccggacctcaagcgaggcaacttcaccgccgacgaggacgacctcatcgtcaagctgcacagcctcctcgggaacaagtggtcgctcatcgccgcgcggctcccggggcggacggacaacgagatcaagaactactggaacacgcacatccggcgcaagctgctgggcagcggcatcgaccccgtcacgcaccgccgcgtcgcggggggcgccgcgaccaccatctcgttccagcccagccccaactccgccgccgccgccgccgccgcagaaacagcagcgcaggcgccgatcaaggccgaggagacggcggccgtcaaggcgcccaggtgccccgacctcaacctggacctctgcatcagcccgccgtgccagcatgaggacgacggcgaggaggaggacgaggagctggacctcaagcccgccttcgtcaagcgggaggcgctgcaggccggccacggccacggccacggcctctgcctcggctgcggcctgggcggacagaagggagcggccgggtgcagctgcagcaacggccaccacttcctggggctcaggaccagcgtgctcgacttcagaggcctggagatgaagtga

SEQ ID NO: 55 - DNA, 828 bp, Zea mays
  source 1..828: mol_type = genomic DNA
atggggaggtcgccgtgctgcgagaaggcgcacaccaacaagggcgcgtggaccaaggaggaggacgagcgcctggtcgcgcacatcagggcgcacggcgaggggtgctggcgctcgctgcccaaggccgccggcctcctgcgctgcggcaagagctgccgcctccgctggatcaactacctccgccccgacctcaagcgcggcaacttcacggaggaagaggacgagctcatcgtcaagctgcacagcgtcctcggcaacaagtggtccctgatcgccggaaggctgcccggcaggacggacaacgagatcaagaactactggaacacgcacatccggaggaagctgctgagcagggggatcgacccggtgacgcaccgcccggtcacggagcaccacgcgtccaacatcaccatatcgttcgagacggaagtggccgccgctgcccgtgatgataagaagggcgccgtcttccggttggaggacgaggaggaggaggagcgcaacaaggcgacgatggtcgtcggccgcgaccggcagagccagagccacagccacagccaccccgccggcgagtggggccaggggaagaggccgctcaagtgccccgacctcaacctggacctctgcatcagcccgccgtgccaggaggaggaggagatggaggaggctgcgatgagagtgagaccggcggtgaagcgggaggccgggctctgcttcggctgcagcctggggctccccaggaccgcggactgcaagtgcagcagcagcagcttcctcgggctcaggaccgccatgctcgacttcagaagcctcgagatgaaatga

SEQ ID NO: 56 - DNA, 771 bp, Panicum virgatum
  source 1..771: mol_type = genomic DNA
atggggcgatcgccgtgctgcgagaaggcgcacacgaacaagggcgcctggaccaaggaggaggacgaccgcctcgttgcctacatccgggcgcacggcgaggggtgctggcgctccctccccaaggccgcgggcctgctgcgctgcggcaagagctgccgcctgcgctggatcaactacctccgcccggacctcaagcgcggcaacttcaccgccgacgaggacgacctcatcgtcaagctccacagcctcctcggcaacaagtggtcgctcatcgccgcgcgcctccccggccgcaccgacaacgagatcaagaactactggaacacgcacatcaagcgcaagctcctcagccgcggcatcgaccccgtcacacaccgccccatcgccgacgcagccagaaacgtcaccatctccttccagcccgacgcgccgtcgcagcagcagctcagcgacgacgccgaggcgccgccgccgccgccgccgcagcagcagcagcagctcaagccgccgcccaggtgccccgacctcaatctcgacctctgcatcagcccgccctgccacaaggaagaagaggaccaggagctcgtcaagcccgccgccgtcaagcgcgagatgctgcaggccggccacggcactctaggactctgcttcggctgcagcctgggcctccagaagggcgccgccgggtgcacctgcagcagcaacagccacttcctggggctcagggtcggcatgctcctcgacttcagaggcctcgagatgaagtga

SEQ ID NO: 57 - DNA, 2496 bp, synthetic construct
  misc_feature 1..2496: note = Construct No. 001
  source 1..2496: mol_type = other DNA
acgtacctcgtgtccaccggtgactctatccccggcgttagaagtgatgatagtctcgttcccaagggaaatcagccttcgaattggaattgatccctccggacattttgtgccgttcgtgtgccagacttgccatccatataatgcatcttcttctttttttcccgcagatggcatgtccgttggtctttcctgtatcatttatttacaaaagaaaaataaattaaacatttattaagttccccccgtaaaaaaaaaatatatatatatatatatatataacacatgcatcataattggtatgtccgtaggtgtttccttatcataactgaaccattggtaaactatcggttccgttaaagcataagactagaaaaggctcggtgcgactcgctaccacgtttctaaagattttatttagcaaattaaccccaatatatattttgctatgagggtctaaacaaactggtatatgagccatttacttaccacttattagttccaagtatttattttttgggttaattaatgtttaaattattggttgacaaaaaatataaaaataatggttaagttattgaaatgacttgagcaatctgatgcaactgcggataacatgaactcattcgaagtgacgtcccaaatatttgattctttgtttttattcctttttgtcaaggtcaagattggccaaacattttcaatatctaaatatattgacattcatagcctggaaaagaaaaaatatatggttaaattagttccaaagtattctagcagcaacaaaaccgctccaataaacgatttccaatttctatctcaaacttgtttcccaatcattagttataatccgtcccctaaaccaaaaaaaaatctaattgtaaaggtgttgcaatagattaaccatttttattttattttggtaaaatagattaaccattttgttagaaaaatgaagtttaaaacatttacagttctacgtgtacatgcttcgaccaatatgcttcgaccaatatggcaaggtctccttgctgtgagaaagaccacacaaacaaaggagcttggactaaggaagaagacgataagctcatctcttacatcaaagctcacggtgaaggttgttggcgttctcttcctagatccgccggtcttcaacgttgcggaaaaagctgtcgtctccgatggattaactatctccgacctgatctcaagaggggtaacttcaccctcgaagaagatgatctcatcatcaaactacatagccttctcggtaacaagtggtctcttattgcgacgagattaccaggaagaacagataacgagattaagaattactggaacacacatgttaagaggaagctattaagaaaagggattgatccggcgactcatcgacctatcaacgagaccaaaacttctcaagattcgtctgattctagtaaaacagaggaccctcttgtcaagattctctcttttggtcctcagctggagaaaatagcaaatttcggggacgagagaattcaaaagagagttgagtactcagttgttgaagaaagatgtctggacttgaatcttgagcttaggatcagtccaccatggcaagacaagctccatgatgagaggaacctaaggtttgggagagtgaagtataggtgcagtgcgtgccgttttggattcgggaacggcaaggagtgtagctgtaataatgtgaaatgtcaaacagaggacagtagtagcagcagttattcttcaaccgacattagtagtagcattggttatgacttcttgggtctaaacaacactagggttttggattttagcactttggaaatgaaatgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 58 - DNA, 2778 bp, synthetic construct
  misc_feature 1..2778: note = Construct No. 002
  source 1..2778: mol_type = other DNA
atacatgtcatgattttataattatgtatatataaatactaattgatgtatgaagtacgtagataatgttacgatctattaatctatttacattaacttttaattagtgttgagtagggaaaattaacatataaacctttagcagttggttgtattattaaaaataatttgaacttaaaatccaccttcgaaaagataaatcaaacaagtataaaaaatgctataaatccagaatatttacctaaggtttttattcttctacttaataatgtaagataaaaccggcacaatacttgttacgtatgcatggtaggtaccgcaattgtgtaagcaaatcggcacaatactaaggttacatatactaactaaataaaacaatctgatttcagtgacaccgtatatctaacctttattcaaatccaagggaacatgacttgacttcttctgttggaactaactcgatccctcaaccatctccagggatagaagagttagtaaaatcaaacttgaagtgaggaagtaagcagtttaacgactccatatgactacagttatatacaaagttgggcacaaagtacaagtactaaatactcaaagtcagataataattttaataagtacaaactatatatatgcagtacaattattgagtatatataaacgagactggtgatttggggcattgtccaccagggtgttatatcccaattgaaatttgaaaatttaagtgtgtgagtgttacgacaaaaaaaagtgtgtgaattgtaggcgcggtgaaaaggtaaattaagattggaactagaaaaatagttgaatatcctttactaaaagttgtcaattccggttttagtaaaaaaaaattttaaaatagaaattttatccaaaagacttcaaacacacatattcgcatatataacataagatatcattttttgtaaacagttaaaaagaaaaacacatgtttttttttttaatttagaaaaaaacatgttattatacaaaacagagttttgcccacttttaatatgttatgaaaagaaaaatgattttcttgggtttggtcagagagattggttgtggtaagaatgggaatcttaattacaaagaattggattttgggtcgacctaccacctaaaacgacgtcgcctccatctctggtttccaaatctctttctcctctccctttataagcttgcgttggccagtcgctcatctcgaaaacagagagaaaaagactaaaaacacagtttaagaagaaggagagatagagagagaagagaaagatagagagggagatggcaaggtctccttgctgtgagaaagaccacacaaacaaaggagcttggactaaggaagaagacgataagctcatctcttacatcaaagctcacggtgaaggttgttggcgttctcttcctagatccgccggtcttcaacgttgcggaaaaagctgtcgtctccgatggattaactatctccgacctgatctcaagaggggtaacttcaccctcgaagaagatgatctcatcatcaaactacatagccttctcggtaacaagtggtctcttattgcgacgagattaccaggaagaacagataacgagattaagaattactggaacacacatgttaagaggaagctattaagaaaagggattgatccggcgactcatcgacctatcaacgagaccaaaacttctcaagattcgtctgattctagtaaaacagaggaccctcttgtcaagattctctcttttggtcctcagctggagaaaatagcaaatttcggggacgagagaattcaaaagagagttgagtactcagttgttgaagaaagatgtctggacttgaatcttgagcttaggatcagtccaccatggcaagacaagctccatgatgagaggaacctaaggtttgggagagtgaagtataggtgcagtgcgtgccgttttggattcgggaacggcaaggagtgtagctgtaataatgtgaaatgtcaaacagaggacagtagtagcagcagttattcttcaaccgacattagtagtagcattggttatgacttcttgggtctaaacaacactagggttttggattttagcactttggaaatgaaatgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 59 - DNA, 2482 bp, synthetic construct
  misc_feature 1..2482: note = Construct No. 003
  source 1..2482: mol_type = other DNA
aactagaacacttcagataaattttgtcgttctgttgacttcatttattctctaaacacaaagaactatagaccataatcgaaataaaaaccctaaaaaccaaatttatctatttaaaacaaacattagctatttgagtttcttttaggtaagttatttaaggttttggagactttaagatgttttcagcatttatggttgtgtcattaatttgtttagtttagtaaagaaagaaaagatagtaattaaagagttggttgtgaaatcatatttaaaacattaataggtatttatgtctaatttggggacaaaatagtggaattctttatcatatctagctagttcttatcgagtttgaactcgggttatgattatgttacatgcattggtccatataaatctatgagcaatcaatataattcgagcattttggtataacataatgagccaagtataacaaaagtatcaaacctatgcaggggagaagatgatgaaaagaagagtgtgagccaatacaaagcagatttgaggacatggcttacaagtcttgggtacagagtttggggagtgatgggtgcacaatggaacagcttctctggttgtccagttcccaagagaaccttcaagctccctaactccatctactatgtcgcctgattaaatcttatttactaacaaaacaataagatcagagtttcattctgattcttgagtcttttttttctctctccctcttttcatttctggtttatataaccaattcaaatgcttatgatccatgcatgaaccatgatcatctttgtgtttttttttccttctgtattaccattttgggcctttgtgaaattgattttgggcttttgttatataatctcctctttctctttctctacctgattggattcaagaacatagccagatttggtaaagtttataagatacaaaatattaagtaagactaaagtagaaatacataataacttgaaagctactctaagttatggcaaggtctccttgctgtgagaaagaccacacaaacaaaggagcttggactaaggaagaagacgataagctcatctcttacatcaaagctcacggtgaaggttgttggcgttctcttcctagatccgccggtcttcaacgttgcggaaaaagctgtcgtctccgatggattaactatctccgacctgatctcaagaggggtaacttcaccctcgaagaagatgatctcatcatcaaactacatagccttctcggtaacaagtggtctcttattgcgacgagattaccaggaagaacagataacgagattaagaattactggaacacacatgttaagaggaagctattaagaaaagggattgatccggcgactcatcgacctatcaacgagaccaaaacttctcaagattcgtctgattctagtaaaacagaggaccctcttgtcaagattctctcttttggtcctcagctggagaaaatagcaaatttcggggacgagagaattcaaaagagagttgagtactcagttgttgaagaaagatgtctggacttgaatcttgagcttaggatcagtccaccatggcaagacaagctccatgatgagaggaacctaaggtttgggagagtgaagtataggtgcagtgcgtgccgttttggattcgggaacggcaaggagtgtagctgtaataatgtgaaatgtcaaacagaggacagtagtagcagcagttattcttcaaccgacattagtagtagcattggttatgacttcttgggtctaaacaacactagggttttggattttagcactttggaaatgaaatgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 60 - DNA, 2546 bp, synthetic construct
  misc_feature 1..2546: note = Construct No. 004
  source 1..2546: mol_type = other DNA
aacccataactttagtattcttcaacccttacaacttatctgagcaaaatcagaaggtcgaatttgatggatggttttgctgtatttggtcaacggttttatttgagacagtagaccagaggaaactcagatgtgatgatgcaaagactgaattggttaagagtgtagattgatttgttctaacattgcaaatgtagagtagaattatgcaaaaaacgttaatgaacagagaagtgattaagcagaaacaaaattagagaagtgatattatatctcaaaatttatttttggtacagctaaagctcaaattgttatagagattagagatattaaaccaaatgacgagtgttttctttagtagtaaacggtgaaaattctcttctgacaaagacaattaaaattttaggtttaagactttaatatttgtcacaaattgtcatttacctaaataaaaaaaaaactaaatattttttttagatacatatgtgtcttataattttaactataaattttaattttatgtcttaaataattgtttacactataaatttaaatattttaatgctaaaattaatttgattcaaaaaagtgattttaattcttatttttcttatagaaagttggtgattgaaaagatttacttaaaaattataacaacttcaatggtgaataacccgacccgaataaaccggatataacaacttcaatgttagcttgatatagaaagtacggtgacgcttaggaggcaagcaagctagtatctgccgctggttagagacaaagaacatgtgtcactcctctcaactaaaactttccttcactttcccgcaaaatcatttcaaaaaagctccaaatttagcttacccatcagctttctcagaaaaccagtgaaagaaacttctcaacttccgatttttcacaatccaccaaactttttttaataactttttttcctcttattacaaaacctccactctcatggcttctcaaacttgttatccatccaaatctcaatccctaattagggttcatttctctgtttctccaaacaggggaattcgaagatggcaaggtctccttgctgtgagaaagaccacacaaacaaaggagcttggactaaggaagaagacgataagctcatctcttacatcaaagctcacggtgaaggttgttggcgttctcttcctagatccgccggtcttcaacgttgcggaaaaagctgtcgtctccgatggattaactatctccgacctgatctcaagaggggtaacttcaccctcgaagaagatgatctcatcatcaaactacatagccttctcggtaacaagtggtctcttattgcgacgagattaccaggaagaacagataacgagattaagaattactggaacacacatgttaagaggaagctattaagaaaagggattgatccggcgactcatcgacctatcaacgagaccaaaacttctcaagattcgtctgattctagtaaaacagaggaccctcttgtcaagattctctcttttggtcctcagctggagaaaatagcaaatttcggggacgagagaattcaaaagagagttgagtactcagttgttgaagaaagatgtctggacttgaatcttgagcttaggatcagtccaccatggcaagacaagctccatgatgagaggaacctaaggtttgggagagtgaagtataggtgcagtgcgtgccgttttggattcgggaacggcaaggagtgtagctgtaataatgtgaaatgtcaaacagaggacagtagtagcagcagttattcttcaaccgacattagtagtagcattggttatgacttcttgggtctaaacaacactagggttttggattttagcactttggaaatgaaatgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 61 - DNA, 2501 bp, synthetic construct
  misc_feature 1..2501: note = Construct No. 005
  source 1..2501: mol_type = other DNA
tcttcttgcatcaatgatatcaacaacaatgggtaataaagaagctacttcgaaattatatattttttcgtattctatattgatcatcagtcttaagtggtttggtttgttgcagtgaagaagaactatgtatggatctacgccaccgttcagttcggttttgtggtccttttcgctcagcttttctacagagttgtaagatttgatgtaatgtcacagagaaaccttactttgttgtcacagagaaaccttactttgttgaagagtttttgattcctcacactctctctcattaacttgtgtgtaggtgaagcagccggtaatgtgcattgtcttagccactatgatcggatttggactcaccatgaccggcacaacagctattaacgagtatttgaaatggaggagaagcaattcccacctgccagaagagccagcaagtactcaggtggtttgacagcagcgtagatcttttgagtgaagctagagtccctaaagggttggatcggttttcaattaaccggtcgggattcggttttcggtttagctttaatcgacttgtctaggttgagatcagatttggttttcaatacttccaagtctttttttttttgccaactaaaatataaggaatgatgataggcacacacatgacacataaaatcataatgaacagtagtatgattagcaatccatatttcttggataacacttcttcacagcttttttgacaggtcactataacacctttttcagttcatttttcattttcaatcctcacccacccaaactctcccttcaaagcaatgtctctcctctctctttctcaattcaaacaaactttattaaacctaaaagaaacatttccaatctctaatgacttagttgatagaatctcatttagttacctagtaataatcttcacactagtaagagaatcctactcttcaccaaactacatctctctctatataacaaaccccaaaacatctcaacatacacacacaacaactacaacaatggcaaggtctccttgctgtgagaaagaccacacaaacaaaggagcttggactaaggaagaagacgataagctcatctcttacatcaaagctcacggtgaaggttgttggcgttctcttcctagatccgccggtcttcaacgttgcggaaaaagctgtcgtctccgatggattaactatctccgacctgatctcaagaggggtaacttcaccctcgaagaagatgatctcatcatcaaactacatagccttctcggtaacaagtggtctcttattgcgacgagattaccaggaagaacagataacgagattaagaattactggaacacacatgttaagaggaagctattaagaaaagggattgatccggcgactcatcgacctatcaacgagaccaaaacttctcaagattcgtctgattctagtaaaacagaggaccctcttgtcaagattctctcttttggtcctcagctggagaaaatagcaaatttcggggacgagagaattcaaaagagagttgagtactcagttgttgaagaaagatgtctggacttgaatcttgagcttaggatcagtccaccatggcaagacaagctccatgatgagaggaacctaaggtttgggagagtgaagtataggtgcagtgcgtgccgttttggattcgggaacggcaaggagtgtagctgtaataatgtgaaatgtcaaacagaggacagtagtagcagcagttattcttcaaccgacattagtagtagcattggttatgacttcttgggtctaaacaacactagggttttggattttagcactttggaaatgaaatgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 62 - DNA, 2575 bp, synthetic construct
  misc_feature 1..2575: note = Construct No. 006
  source 1..2575: mol_type = other DNA
tgcgaacagtttgattctgtttttctttttcctttttttgggtaattttcttataacttttttcatagtttcgattatttggataaaattttcagattgaggatcattttatttatttattagtgtagtctaatttagttgtataactataaaattgttgtttgtttccgaatcataagttttttttttttttggttttgtattgataggtgcaagagactcaaaattctggtttcgatgttaacagaattcaagtagctgcccacttgattcgatttgttttgtatttggaaacaaccatggctggtcaaggcccagcccgttgtgcttctgaacctgcctagtcccatggactagatctttatccgcagactccaaaagaaaaaggattggcgcagaggaattgtcatggaaacagaatgaacaagaaagggtgaagaagatcaaaggcatatatgatctttacattctctttagcttatgtatgcagaaaattcacctaattaaggacagggaacgtaacttggcttgcactcctctcaccaaaccttaccccctaactaattttaattcaaaattactagtattttggccgatcactttatataataagataccagatttattatatttacgaattatcagcatgcatatactgtatatagttttttttttgttaaagggtaaaataataggatccttttgaataaaatgaacatatataattagtataatgaaaacagaaggaaatgagattaggacagtaagtaaaatgagagagacctgcaaaggataaaaaagagaagcttaaggaaaccgcgacgatgaaagaaagacatgtcatcagctgatggatgtgagtgatgagtttgttgcagttgtgtagaaatttttactaaaacagttgtttttacaaaaaagaaataatataaaacgaaagcttagcttgaaggcaatggagactctacaacaaactatgtaccatacagagagagaaactaaaagcttttcacacataaaaaccaaacttattcgtctctcattgatcaccgttttgttctctcaagatcgctgctaatctccggccgtccctatggcaaggtctccttgctgtgagaaagaccacacaaacaaaggagcttggactaaggaagaagacgataagctcatctcttacatcaaagctcacggtgaaggttgttggcgttctcttcctagatccgccggtcttcaacgttgcggaaaaagctgtcgtctccgatggattaactatctccgacctgatctcaagaggggtaacttcaccctcgaagaagatgatctcatcatcaaactacatagccttctcggtaacaagtggtctcttattgcgacgagattaccaggaagaacagataacgagattaagaattactggaacacacatgttaagaggaagctattaagaaaagggattgatccggcgactcatcgacctatcaacgagaccaaaacttctcaagattcgtctgattctagtaaaacagaggaccctcttgtcaagattctctcttttggtcctcagctggagaaaatagcaaatttcggggacgagagaattcaaaagagagttgagtactcagttgttgaagaaagatgtctggacttgaatcttgagcttaggatcagtccaccatggcaagacaagctccatgatgagaggaacctaaggtttgggagagtgaagtataggtgcagtgcgtgccgttttggattcgggaacggcaaggagtgtagctgtaataatgtgaaatgtcaaacagaggacagtagtagcagcagttattcttcaaccgacattagtagtagcattggttatgacttcttgggtctaaacaacactagggttttggattttagcactttggaaatgaaatgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 63 - DNA, 2628 bp, synthetic construct
  misc_feature 1..2628: note = Construct No. 007
  source 1..2628: mol_type = other DNA
tctctaattgtcaagtatcttagtctagagttaattacttaaatactaaaaggctgtcgacaaaatcaagcttgaatctccttgtggtatcttcaactcttcgttgtctgcttacgagtggtttactcagtaattatctataatatgttattttttttccctcatcttttagttgttgtttcattacattgaaaagcttgtaatgtctttatatggtatatatggatcttatgagtgaggcaagatccatgatgtttttgatcttagaatgtatatgatgatcttagaatgtatttgaccgcccacaaattattgttcattgggattatatctctagtccaactccaagcaatcgaaatgggtcctgcttttaagaacaacagtatatgtttaagaataataactttatatattctcgattttaagatcttttgacaaaacctccttttcgttaggagcgtactaatttccaagtgtttgattagtggggtctccgtaaatttatttagagtttctatctatttattaatagctcaattaattaatctatactgtatctaaacatcaatttatatatttactcttgagaccaaaactgtcaatttataacattggatagtttcttaattcttattatatatttttcaaacacttttcaagactaatctccacattaggtactctctctagagataaaaatatttatcaaaaacatttttatttatttattaagtagtagataaactactgtggcaaaatcgtaaatgtctaaatgctgatgaattttttttgctgctccaatctggtttagtgctccatatacatccacggccaaaatgaatctatggcggcattaagattcattagtaagcaacgattatattaatataattgtttttagcaatgattttccgtaatttcccaaatatgtttcagttaatgtgttccaatcccaacaactggttgttgcaaaagaccaccaacgcaagcaatcatcaaacatcaaaataatcttaccttagcgaacaaacaataactacacaattctcataaagctcttatatatcactaacttcacacattttgttttccacaaaaataaaaacggaactcactcaagaaaccttcttccttgaagagagggttatggcaaggtctccttgctgtgagaaagaccacacaaacaaaggagcttggactaaggaagaagacgataagctcatctcttacatcaaagctcacggtgaaggttgttggcgttctcttcctagatccgccggtcttcaacgttgcggaaaaagctgtcgtctccgatggattaactatctccgacctgatctcaagaggggtaacttcaccctcgaagaagatgatctcatcatcaaactacatagccttctcggtaacaagtggtctcttattgcgacgagattaccaggaagaacagataacgagattaagaattactggaacacacatgttaagaggaagctattaagaaaagggattgatccggcgactcatcgacctatcaacgagaccaaaacttctcaagattcgtctgattctagtaaaacagaggaccctcttgtcaagattctctcttttggtcctcagctggagaaaatagcaaatttcggggacgagagaattcaaaagagagttgagtactcagttgttgaagaaagatgtctggacttgaatcttgagcttaggatcagtccaccatggcaagacaagctccatgatgagaggaacctaaggtttgggagagtgaagtataggtgcagtgcgtgccgttttggattcgggaacggcaaggagtgtagctgtaataatgtgaaatgtcaaacagaggacagtagtagcagcagttattcttcaaccgacattagtagtagcattggttatgacttcttgggtctaaacaacactagggttttggattttagcactttggaaatgaaatgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 64 - DNA, 2506 bp, synthetic construct
  misc_feature 1..2506: note = Construct No. 008
  source 1..2506: mol_type = other DNA
aactagaacacttcagataaattttgtcgttctgttgacttcatttattctctaaacacaaagaactatagaccataatcgaaataaaaaccctaaaaaccaaatttatctatttaaaacaaacattagctatttgagtttcttttaggtaagttatttaaggttttggagactttaagatgttttcagcatttatggttgtgtcattaatttgtttagtttagtaaagaaagaaaagatagtaattaaagagttggttgtgaaatcatatttaaaacattaataggtatttatgtctaatttggggacaaaatagtggaattctttatcatatctagctagttcttatcgagtttgaactcgggttatgattatgttacatgcattggtccatataaatctatgagcaatcaatataattcgagcattttggtataacataatgagccaagtataacaaaagtatcaaacctatgcaggggagaagatgatgaaaagaagagtgtgagccaatacaaagcagatttgaggacatggcttacaagtcttgggtacagagtttggggagtgatgggtgcacaatggaacagcttctctggttgtccagttcccaagagaaccttcaagctccctaactccatctactatgtcgcctgattaaatcttatttactaacaaaacaataagatcagagtttcattctgattcttgagtcttttttttctctctccctcttttcatttctggtttatataaccaattcaaatgcttatgatccatgcatgaaccatgatcatctttgtgtttttttttccttctgtattaccattttgggcctttgtgaaattgattttgggcttttgttatataatctcctctttctctttctctacctgattggattcaagaacatagccagatttggtaaagtttataagatacaaaatattaagtaagactaaagtagaaatacataataacttgaaagctactctaagttatgggaaggtcaccgtgctgtgagaaagctcacacaaacaaaggagcatggacgaaagaagaggacgagaggctcgtcgcctacattaaagctcatggagaaggctgctggagatctctccccaaagccgccggacttcttcgctgtggcaagagctgccgtctccggtggatcaactatctccggcctgaccttaagcgtggaaacttcaccgaggaagaagacgaactcatcatcaagctccatagccttcttggcaacaaatggtcgcttattgccgggagattaccgggaagaacagataacgagataaagaactattggaacacgcatatacgaagaaagcttataaacagagggattgatccaacgagtcatagaccaatccaagaatcatcagcttctcaagattctaaacctacacaactagaaccagttacgagtaataccattaatatctcattcacttctgctccaaaggtcgaaacgttccatgaaagtataagctttccgggaaaatcagagaaaatctcaatgcttacgttcaaagaagaaaaagatgagtgcccagttcaagaaaagttcccagatttgaatcttgagctcagaatcagtcttcctgatgatgttgatcgtcttcaagggcatggaaagtcaacaacgccacgttgtttcaagtgcagcttagggatgataaacggcatggagtgcagatgcggaagaatgagatgcgatgtagtcggaggtagcagcaaggggagtgacatgagcaatggatttgattttttagggttggcaaagaaagagaccacttctcttttgggctttcgaagcttggagatgaaataacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 65 - DNA, 2570 bp, synthetic construct
  misc_feature 1..2570: note = Construct No. 009
  source 1..2570: mol_type = other DNA
aacccataactttagtattcttcaacccttacaacttatctgagcaaaatcagaaggtcgaatttgatggatggttttgctgtatttggtcaacggttttatttgagacagtagaccagaggaaactcagatgtgatgatgcaaagactgaattggttaagagtgtagattgatttgttctaacattgcaaatgtagagtagaattatgcaaaaaacgttaatgaacagagaagtgattaagcagaaacaaaattagagaagtgatattatatctcaaaatttatttttggtacagctaaagctcaaattgttatagagattagagatattaaaccaaatgacgagtgttttctttagtagtaaacggtgaaaattctcttctgacaaagacaattaaaattttaggtttaagactttaatatttgtcacaaattgtcatttacctaaataaaaaaaaaactaaatattttttttagatacatatgtgtcttataattttaactataaattttaattttatgtcttaaataattgtttacactataaatttaaatattttaatgctaaaattaatttgattcaaaaaagtgattttaattcttatttttcttatagaaagttggtgattgaaaagatttacttaaaaattataacaacttcaatggtgaataacccgacccgaataaaccggatataacaacttcaatgttagcttgatatagaaagtacggtgacgcttaggaggcaagcaagctagtatctgccgctggttagagacaaagaacatgtgtcactcctctcaactaaaactttccttcactttcccgcaaaatcatttcaaaaaagctccaaatttagcttacccatcagctttctcagaaaaccagtgaaagaaacttctcaacttccgatttttcacaatccaccaaactttttttaataactttttttcctcttattacaaaacctccactctcatggcttctcaaacttgttatccatccaaatctcaatccctaattagggttcatttctctgtttctccaaacaggggaattcgaagatgggaaggtcaccgtgctgtgagaaagctcacacaaacaaaggagcatggacgaaagaagaggacgagaggctcgtcgcctacattaaagctcatggagaaggctgctggagatctctccccaaagccgccggacttcttcgctgtggcaagagctgccgtctccggtggatcaactatctccggcctgaccttaagcgtggaaacttcaccgaggaagaagacgaactcatcatcaagctccatagccttcttggcaacaaatggtcgcttattgccgggagattaccgggaagaacagataacgagataaagaactattggaacacgcatatacgaagaaagcttataaacagagggattgatccaacgagtcatagaccaatccaagaatcatcagcttctcaagattctaaacctacacaactagaaccagttacgagtaataccattaatatctcattcacttctgctccaaaggtcgaaacgttccatgaaagtataagctttccgggaaaatcagagaaaatctcaatgcttacgttcaaagaagaaaaagatgagtgcccagttcaagaaaagttcccagatttgaatcttgagctcagaatcagtcttcctgatgatgttgatcgtcttcaagggcatggaaagtcaacaacgccacgttgtttcaagtgcagcttagggatgataaacggcatggagtgcagatgcggaagaatgagatgcgatgtagtcggaggtagcagcaaggggagtgacatgagcaatggatttgattttttagggttggcaaagaaagagaccacttctcttttgggctttcgaagcttggagatgaaataacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 66 - DNA, 2485 bp, synthetic construct
  misc_feature 1..2485: note = Construct No. 010
  source 1..2485: mol_type = other DNA
aggcggggccggaggagggcaccagagaggctgctcaggagagagaaataatagaatatgtggtatagagtaaacatgagtgcggatgattgtggtatagagtaaagaattttgctgactaggacagaatattctttttagggtagaaatttagagtactatgagtgcggatagcctaaggaccactttaaatttgacacaatattgaaatttgaatggttttaacatttgaaggctgaaaaccaaaatactttgtagctaagtgttggaaacccgactcggccaataagtcgacagaccgtaaaataaggtcaatctaaactttatgataaatattcttgtttgatagcaatagcattgcaggaccaggacccaagggaagagaagatgccaaatcccatcgaggctaaagcaaaaacgatccaatttatgagcaaacccacactgaagtttcaaaattgttttctgaaaaaaaagtaaccagcaagttaaaaaatgagatggcgggaaagccaagtctcggttggtcgaggggttggttggggcgcagcctgacaagtgacaacggcagcaggatagtagcatcaggcgcaagccagcgcaggcggcagcgcgaggatttcgcttcacttagcggcaacggagacgctgcacccaaccaacacgagctccccctcacccgctgcgacgcgcgcgtcccacgagcggaagccccccgcgccgacgcgagcgcgggggctcgaccgaccgacccaacgcctccatctccaccgcgcgcaccaaatcgcactcccgtccgccccgccgatcgaacagccaccgctcacctctcccacccgccaaaaacctccggcctcctctcatattcatatagctagcccctgccacaaggtagagcgtcgctcacacctgcgtcgccctgcctcgcaatcgcgaatctgtcgagcacctgaggggtcggaggccgagagctagcctagcacgccggcctccgcgcgcgatggggaggtcgccgtgctgcgagaaggcgcacaccaacaagggcgcgtggaccaaggaggaggacgagcgcctggtcgcgcacatcagggcgcacggcgaggggtgctggcgctcgctgcccaaggccgccggcctcctgcgctgcggcaagagctgccgcctccgctggatcaactacctccgccccgacctcaagcgcggcaacttcacggaggaagaggacgagctcatcgtcaagctgcacagcgtcctcggcaacaagtggtccctgatcgccggaaggctgcccggcaggacggacaacgagatcaagaactactggaacacgcacatccggaggaagctgctgagcagggggatcgacccggtgacgcaccgcccggtcacggagcaccacgcgtccaacatcaccatatcgttcgagacggaagtggccgccgctgcccgtgatgataagaagggcgccgtcttccggttggaggacgaggaggaggaggagcgcaacaaggcgacgatggtcgtcggccgcgaccggcagagccagagccacagccacagccaccccgccggcgagtggggccaggggaagaggccgctcaagtgccccgacctcaacctggacctctgcatcagcccgccgtgccaggaggaggaggagatggaggaggctgcgatgagagtgagaccggcggtgaagcgggaggccgggctctgcttcggctgcagcctggggctccccaggaccgcggactgcaagtgcagcagcagcagcttcctcgggctcaggaccgccatgctcgacttcagaagcctcgagatgaaatgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 67 - DNA, 2485 bp, synthetic construct
  misc_feature 1..2485: note = Construct No. 011
  source 1..2485: mol_type = other DNA
atactgaacattatgttgcataacatgtagataaggacacgaaaacatagaaagtttctcagttatatttacccatcaacatgaaataaaaaacaacaaagatgtcatagtgatgtttgtttcaacttaccaagggtgaccatgtcgtatttataataatattatatatttatatcgtcaatagaatattagtgttacggtgatattttagcacaccgattttttatatcatactgatgtttatcgttttgtatctatattttatatttgttttataataatattagatatttatttcgtcaatagaatattaatgttatgatgatactttactatattgattttacatatgatagtgatgttactccttccgtatctatattttatattagtttttatctcctggcaacacggtcacaacagaagagaagtttttcagaccgattccaggatcgattttttttttatatctgggctaagacatcaggtagagattgtttaacctttgcggctttccgcactgacggacccacccccaccgcatcaacggaacctaccaaccacccccgtgctccgaccccccatctgcccgtcttccaggttacgccccgcgcggccgcgcgcgcggaagctgtatcaccccacccgtcgacgtcgtcttcgcttcgaaaccccgcaaaaccccgcggaaaaaacccacctgctgcacgcacgcaccccctccctctccctccccatggcgcctcccctcacccaactctttgcttccattctttccatccacccgccaatgcgacgccgacgccgcaactccacccaccgcctgccagcgccacctcaccgcaccgcttccatcaccccgcgatcatgggctaccgctatatcaccacgcctccaacctccggcacgcttagcctctctctcccattctctcacacccaacacccagctatcacaccctgatccccgaggccgcgcgtcggggtgaggaggaggggccatggggaggtcgccgtgctgcgagaaggcgcacaccaacaagggcgcgtggaccaaggaggaggacgagcgcctggtcgcgcacatcagggcgcacggcgaggggtgctggcgctcgctgcccaaggccgccggcctcctgcgctgcggcaagagctgccgcctccgctggatcaactacctccgccccgacctcaagcgcggcaacttcacggaggaagaggacgagctcatcgtcaagctgcacagcgtcctcggcaacaagtggtccctgatcgccggaaggctgcccggcaggacggacaacgagatcaagaactactggaacacgcacatccggaggaagctgctgagcagggggatcgacccggtgacgcaccgcccggtcacggagcaccacgcgtccaacatcaccatatcgttcgagacggaagtggccgccgctgcccgtgatgataagaagggcgccgtcttccggttggaggacgaggaggaggaggagcgcaacaaggcgacgatggtcgtcggccgcgaccggcagagccagagccacagccacagccaccccgccggcgagtggggccaggggaagaggccgctcaagtgccccgacctcaacctggacctctgcatcagcccgccgtgccaggaggaggaggagatggaggaggctgcgatgagagtgagaccggcggtgaagcgggaggccgggctctgcttcggctgcagcctggggctccccaggaccgcggactgcaagtgcagcagcagcagcttcctcgggctcaggaccgccatgctcgacttcagaagcctcgagatgaaatgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 68 - DNA, 2626 bp, synthetic construct
  misc_feature 1..2626: note = Construct No. 012
  source 1..2626: mol_type = other DNA
gagttaaattgatatggttttgtctgtcaaggtgccgtttctatggttggaggaggaaaatgaaattaggggaatagaacagtcgcaatccaaaagctattgttctctcttctacggtagttgatagttcgatacgtgtgtttgatgtgagagactgagaggagtgcacggtgcacctgccccgtaaggacgcggaactacattatcagaggctaggccggtactgttatacgcgacgttcacgaatcacgatgcgtaaaaaagtgaagcatgaaacagtactaggctctccacgggccacatcatgaaaaaactggtgcgacgccacgcttaacaatttgtcggtcgtaaactcgtaaagttaaaagccccacgacattgtcaaccaatatctcagcacatgaacttctctaacgagtacaacgaaaccgcatccgcaaaagcgccgtgaaccaaagctcttgccgtgctgtgccgtgccggtggccgtgaacatgtggaacacgaagaactcttgcgcgagatcggagcacctgacctcccacctcgcgtccggcccgtcgccgtcgcagcaagcgggctgtcaaaaacgacgccacagcgcgagcgctctcgccgatccggcggacccaccctcctcacctcgcgcaccaaccgcccgttcgctagtccgatcccccacccctcatcccccctacgccttgcaggttacgcgcctcgccgcggccaacgcaaaccaaaccaaatcccccgtcaccttcgcttcgaaaccccgcaaaaccccatggaagaaaacaccgaacacctgccgcgcgcacgcctcctcctcccccgcctctcctcctctctcccgttccatgtccgctcaaccttgcttccattctttccatccacccgccgatcgacgcgatgccgacgccccaaccccacccaccgcctgccagcgccaccccacctcgcgcctctgcggctatggctatatcaccatgcctccaacctccggtacgcttagcctctctctctctccccctcccattctgcgcattgctctctgcgcgcggtcgcgtgctgctgctcgcggcgccccggagcgtctcctttgggggagaggagaggagaggagaggagaggggggtgagccatggggaggtcgccgtgctgcgagaaggcgcacaccaacaagggcgcgtggaccaaggaggaggacgagcgcctggtcgcgcacatcagggcgcacggcgaggggtgctggcgctcgctgcccaaggccgccggcctcctgcgctgcggcaagagctgccgcctccgctggatcaactacctccgccccgacctcaagcgcggcaacttcacggaggaagaggacgagctcatcgtcaagctgcacagcgtcctcggcaacaagtggtccctgatcgccggaaggctgcccggcaggacggacaacgagatcaagaactactggaacacgcacatccggaggaagctgctgagcagggggatcgacccggtgacgcaccgcccggtcacggagcaccacgcgtccaacatcaccatatcgttcgagacggaagtggccgccgctgcccgtgatgataagaagggcgccgtcttccggttggaggacgaggaggaggaggagcgcaacaaggcgacgatggtcgtcggccgcgaccggcagagccagagccacagccacagccaccccgccggcgagtggggccaggggaagaggccgctcaagtgccccgacctcaacctggacctctgcatcagcccgccgtgccaggaggaggaggagatggaggaggctgcgatgagagtgagaccggcggtgaagcgggaggccgggctctgcttcggctgcagcctggggctccccaggaccgcggactgcaagtgcagcagcagcagcttcctcgggctcaggaccgccatgctcgacttcagaagcctcgagatgaaatgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 69 - DNA, 2485 bp, synthetic construct
  misc_feature 1..2485: note = Construct No. 013
  source 1..2485: mol_type = other DNA
ttcaatgcaggatgacgccaagagggagaaaccaagcagaggtggacggtacaacgtgtaagtcaccgcaaaacgttgcagcttggatagtggccatcgagtggtgtgccgataccggcgcctgttctttacagcctcagctagtgttgttgtccgaggcaatttttccgacctattgtgttgctttcctctctgatagcttatggtaaaagatacaaagatgttgaggagtttgtacgccacttaattttgctcgtaacatacattgacaatcaagaggagccatggcattgcgatctgcttacacggcatattcttactggatggtgtacactacttaccctttttaatgcaagcatcaatccattgcttttctcactgcacacctgattcgtactgaaaacgtgaaacataaaaaaaaacaaaaatctagctgatgttggctctcggggcctcgagtctagtttgtcctagatggctaacctgatatgtgttggtcacgctcacgtttgaaccgagaaagagtgtgtgtgtgtgtgtgtcggcgtgctgctacaccagagcctccctgaatcgcaatgcgtgttaacgccagcatcgcaggatttcatctcacttgacaggttcagatggccttcctcctaccgtctgccatttatacacgcagtgacttaacgcttacacgagccggatggcccggatctcccccctgcaccatctcaccagaaaaacggtgaggcgtcaccgcaacccacccaccaaacacatccacgtcccttcaccgttggccttcgattttgcttcagctgcactacgacccctccaacacatttccctcgcgtctcgttgcgatctcaccttacgacgatctcgttccagcagcagcagcatcggcagcggcggcttgcttccgaagcgagcaatgcatggcgcgcgcggccgcgtgcgtgcgtgccttggcttgcgctctaatcaaaccgggacgccccaactcacggttatggggaggtcgccgtgctgcgagaaggcgcacaccaacaagggcgcgtggaccaaggaggaggacgagcgcctggtcgcgcacatcagggcgcacggcgaggggtgctggcgctcgctgcccaaggccgccggcctcctgcgctgcggcaagagctgccgcctccgctggatcaactacctccgccccgacctcaagcgcggcaacttcacggaggaagaggacgagctcatcgtcaagctgcacagcgtcctcggcaacaagtggtccctgatcgccggaaggctgcccggcaggacggacaacgagatcaagaactactggaacacgcacatccggaggaagctgctgagcagggggatcgacccggtgacgcaccgcccggtcacggagcaccacgcgtccaacatcaccatatcgttcgagacggaagtggccgccgctgcccgtgatgataagaagggcgccgtcttccggttggaggacgaggaggaggaggagcgcaacaaggcgacgatggtcgtcggccgcgaccggcagagccagagccacagccacagccaccccgccggcgagtggggccaggggaagaggccgctcaagtgccccgacctcaacctggacctctgcatcagcccgccgtgccaggaggaggaggagatggaggaggctgcgatgagagtgagaccggcggtgaagcgggaggccgggctctgcttcggctgcagcctggggctccccaggaccgcggactgcaagtgcagcagcagcagcttcctcgggctcaggaccgccatgctcgacttcagaagcctcgagatgaaatgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 70 - DNA, 2440 bp, synthetic construct
  misc_feature 1..2440: note = Construct No. 014
  source 1..2440: mol_type = other DNA
aggcggggccggaggagggcaccagagaggctgctcaggagagagaaataatagaatatgtggtatagagtaaacatgagtgcggatgattgtggtatagagtaaagaattttgctgactaggacagaatattctttttagggtagaaatttagagtactatgagtgcggatagcctaaggaccactttaaatttgacacaatattgaaatttgaatggttttaacatttgaaggctgaaaaccaaaatactttgtagctaagtgttggaaacccgactcggccaataagtcgacagaccgtaaaataaggtcaatctaaactttatgataaatattcttgtttgatagcaatagcattgcaggaccaggacccaagggaagagaagatgccaaatcccatcgaggctaaagcaaaaacgatccaatttatgagcaaacccacactgaagtttcaaaattgttttctgaaaaaaaagtaaccagcaagttaaaaaatgagatggcgggaaagccaagtctcggttggtcgaggggttggttggggcgcagcctgacaagtgacaacggcagcaggatagtagcatcaggcgcaagccagcgcaggcggcagcgcgaggatttcgcttcacttagcggcaacggagacgctgcacccaaccaacacgagctccccctcacccgctgcgacgcgcgcgtcccacgagcggaagccccccgcgccgacgcgagcgcgggggctcgaccgaccgacccaacgcctccatctccaccgcgcgcaccaaatcgcactcccgtccgccccgccgatcgaacagccaccgctcacctctcccacccgccaaaaacctccggcctcctctcatattcatatagctagcccctgccacaaggtagagcgtcgctcacacctgcgtcgccctgcctcgcaatcgcgaatctgtcgagcacctgaggggtcggaggccgagagctagcctagcacgccggcctccgcgcgcgatggggcggtcgccgtgctgcgagaaggcgcacaccaacaggggcgcgtggaccaaggaggaggacgagcggctggtggcctacgtccgcgcgcacggcgaagggtgctggcgctcgctgcccagggcggcgggcctgctgcgctgcggcaagagctgccgcctgcgctggatcaactacctccgcccggacctcaagcgaggcaacttcaccgccgacgaggacgacctcatcgtcaagctgcacagcctcctcgggaacaagtggtcgctcatcgccgcgcggctcccggggcggacggacaacgagatcaagaactactggaacacgcacatccggcgcaagctgctgggcagcggcatcgaccccgtcacgcaccgccgcgtcgcggggggcgccgcgaccaccatctcgttccagcccagccccaactccgccgccgccgccgccgccgcagaaacagcagcgcaggcgccgatcaaggccgaggagacggcggccgtcaaggcgcccaggtgccccgacctcaacctggacctctgcatcagcccgccgtgccagcatgaggacgacggcgaggaggaggacgaggagctggacctcaagcccgccttcgtcaagcgggaggcgctgcaggccggccacggccacggccacggcctctgcctcggctgcggcctgggcggacagaagggagcggccgggtgcagctgcagcaacggccaccacttcctggggctcaggaccagcgtgctcgacttcagaggcctggagatgaagtgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 71 - DNA, 2440 bp, synthetic construct
  misc_feature 1..2440: note = Construct No. 015
  source 1..2440: mol_type = other DNA
atactgaacattatgttgcataacatgtagataaggacacgaaaacatagaaagtttctcagttatatttacccatcaacatgaaataaaaaacaacaaagatgtcatagtgatgtttgtttcaacttaccaagggtgaccatgtcgtatttataataatattatatatttatatcgtcaatagaatattagtgttacggtgatattttagcacaccgattttttatatcatactgatgtttatcgttttgtatctatattttatatttgttttataataatattagatatttatttcgtcaatagaatattaatgttatgatgatactttactatattgattttacatatgatagtgatgttactccttccgtatctatattttatattagtttttatctcctggcaacacggtcacaacagaagagaagtttttcagaccgattccaggatcgattttttttttatatctgggctaagacatcaggtagagattgtttaacctttgcggctttccgcactgacggacccacccccaccgcatcaacggaacctaccaaccacccccgtgctccgaccccccatctgcccgtcttccaggttacgccccgcgcggccgcgcgcgcggaagctgtatcaccccacccgtcgacgtcgtcttcgcttcgaaaccccgcaaaaccccgcggaaaaaacccacctgctgcacgcacgcaccccctccctctccctccccatggcgcctcccctcacccaactctttgcttccattctttccatccacccgccaatgcgacgccgacgccgcaactccacccaccgcctgccagcgccacctcaccgcaccgcttccatcaccccgcgatcatgggctaccgctatatcaccacgcctccaacctccggcacgcttagcctctctctcccattctctcacacccaacacccagctatcacaccctgatccccgaggccgcgcgtcggggtgaggaggaggggccatggggcggtcgccgtgctgcgagaaggcgcacaccaacaggggcgcgtggaccaaggaggaggacgagcggctggtggcctacgtccgcgcgcacggcgaagggtgctggcgctcgctgcccagggcggcgggcctgctgcgctgcggcaagagctgccgcctgcgctggatcaactacctccgcccggacctcaagcgaggcaacttcaccgccgacgaggacgacctcatcgtcaagctgcacagcctcctcgggaacaagtggtcgctcatcgccgcgcggctcccggggcggacggacaacgagatcaagaactactggaacacgcacatccggcgcaagctgctgggcagcggcatcgaccccgtcacgcaccgccgcgtcgcggggggcgccgcgaccaccatctcgttccagcccagccccaactccgccgccgccgccgccgccgcagaaacagcagcgcaggcgccgatcaaggccgaggagacggcggccgtcaaggcgcccaggtgccccgacctcaacctggacctctgcatcagcccgccgtgccagcatgaggacgacggcgaggaggaggacgaggagctggacctcaagcccgccttcgtcaagcgggaggcgctgcaggccggccacggccacggccacggcctctgcctcggctgcggcctgggcggacagaagggagcggccgggtgcagctgcagcaacggccaccacttcctggggctcaggaccagcgtgctcgacttcagaggcctggagatgaagtgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 72 - DNA, 2581 bp, synthetic construct
  misc_feature 1..2581: note = Construct No. 016
  source 1..2581: mol_type = other DNA
gagttaaattgatatggttttgtctgtcaaggtgccgtttctatggttggaggaggaaaatgaaattaggggaatagaacagtcgcaatccaaaagctattgttctctcttctacggtagttgatagttcgatacgtgtgtttgatgtgagagactgagaggagtgcacggtgcacctgccccgtaaggacgcggaactacattatcagaggctaggccggtactgttatacgcgacgttcacgaatcacgatgcgtaaaaaagtgaagcatgaaacagtactaggctctccacgggccacatcatgaaaaaactggtgcgacgccacgcttaacaatttgtcggtcgtaaactcgtaaagttaaaagccccacgacattgtcaaccaatatctcagcacatgaacttctctaacgagtacaacgaaaccgcatccgcaaaagcgccgtgaaccaaagctcttgccgtgctgtgccgtgccggtggccgtgaacatgtggaacacgaagaactcttgcgcgagatcggagcacctgacctcccacctcgcgtccggcccgtcgccgtcgcagcaagcgggctgtcaaaaacgacgccacagcgcgagcgctctcgccgatccggcggacccaccctcctcacctcgcgcaccaaccgcccgttcgctagtccgatcccccacccctcatcccccctacgccttgcaggttacgcgcctcgccgcggccaacgcaaaccaaaccaaatcccccgtcaccttcgcttcgaaaccccgcaaaaccccatggaagaaaacaccgaacacctgccgcgcgcacgcctcctcctcccccgcctctcctcctctctcccgttccatgtccgctcaaccttgcttccattctttccatccacccgccgatcgacgcgatgccgacgccccaaccccacccaccgcctgccagcgccaccccacctcgcgcctctgcggctatggctatatcaccatgcctccaacctccggtacgcttagcctctctctctctccccctcccattctgcgcattgctctctgcgcgcggtcgcgtgctgctgctcgcggcgccccggagcgtctcctttgggggagaggagaggagaggagaggagaggggggtgagccatggggcggtcgccgtgctgcgagaaggcgcacaccaacaggggcgcgtggaccaaggaggaggacgagcggctggtggcctacgtccgcgcgcacggcgaagggtgctggcgctcgctgcccagggcggcgggcctgctgcgctgcggcaagagctgccgcctgcgctggatcaactacctccgcccggacctcaagcgaggcaacttcaccgccgacgaggacgacctcatcgtcaagctgcacagcctcctcgggaacaagtggtcgctcatcgccgcgcggctcccggggcggacggacaacgagatcaagaactactggaacacgcacatccggcgcaagctgctgggcagcggcatcgaccccgtcacgcaccgccgcgtcgcggggggcgccgcgaccaccatctcgttccagcccagccccaactccgccgccgccgccgccgccgcagaaacagcagcgcaggcgccgatcaaggccgaggagacggcggccgtcaaggcgcccaggtgccccgacctcaacctggacctctgcatcagcccgccgtgccagcatgaggacgacggcgaggaggaggacgaggagctggacctcaagcccgccttcgtcaagcgggaggcgctgcaggccggccacggccacggccacggcctctgcctcggctgcggcctgggcggacagaagggagcggccgggtgcagctgcagcaacggccaccacttcctggggctcaggaccagcgtgctcgacttcagaggcctggagatgaagtgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 73 - DNA, 2440 bp, synthetic construct
  misc_feature 1..2440: note = Construct No. 017
  source 1..2440: mol_type = other DNA
ttcaatgcaggatgacgccaagagggagaaaccaagcagaggtggacggtacaacgtgtaagtcaccgcaaaacgttgcagcttggatagtggccatcgagtggtgtgccgataccggcgcctgttctttacagcctcagctagtgttgttgtccgaggcaatttttccgacctattgtgttgctttcctctctgatagcttatggtaaaagatacaaagatgttgaggagtttgtacgccacttaattttgctcgtaacatacattgacaatcaagaggagccatggcattgcgatctgcttacacggcatattcttactggatggtgtacactacttaccctttttaatgcaagcatcaatccattgcttttctcactgcacacctgattcgtactgaaaacgtgaaacataaaaaaaaacaaaaatctagctgatgttggctctcggggcctcgagtctagtttgtcctagatggctaacctgatatgtgttggtcacgctcacgtttgaaccgagaaagagtgtgtgtgtgtgtgtgtcggcgtgctgctacaccagagcctccctgaatcgcaatgcgtgttaacgccagcatcgcaggatttcatctcacttgacaggttcagatggccttcctcctaccgtctgccatttatacacgcagtgacttaacgcttacacgagccggatggcccggatctcccccctgcaccatctcaccagaaaaacggtgaggcgtcaccgcaacccacccaccaaacacatccacgtcccttcaccgttggccttcgattttgcttcagctgcactacgacccctccaacacatttccctcgcgtctcgttgcgatctcaccttacgacgatctcgttccagcagcagcagcatcggcagcggcggcttgcttccgaagcgagcaatgcatggcgcgcgcggccgcgtgcgtgcgtgccttggcttgcgctctaatcaaaccgggacgccccaactcacggttatggggcggtcgccgtgctgcgagaaggcgcacaccaacaggggcgcgtggaccaaggaggaggacgagcggctggtggcctacgtccgcgcgcacggcgaagggtgctggcgctcgctgcccagggcggcgggcctgctgcgctgcggcaagagctgccgcctgcgctggatcaactacctccgcccggacctcaagcgaggcaacttcaccgccgacgaggacgacctcatcgtcaagctgcacagcctcctcgggaacaagtggtcgctcatcgccgcgcggctcccggggcggacggacaacgagatcaagaactactggaacacgcacatccggcgcaagctgctgggcagcggcatcgaccccgtcacgcaccgccgcgtcgcggggggcgccgcgaccaccatctcgttccagcccagccccaactccgccgccgccgccgccgccgcagaaacagcagcgcaggcgccgatcaaggccgaggagacggcggccgtcaaggcgcccaggtgccccgacctcaacctggacctctgcatcagcccgccgtgccagcatgaggacgacggcgaggaggaggacgaggagctggacctcaagcccgccttcgtcaagcgggaggcgctgcaggccggccacggccacggccacggcctctgcctcggctgcggcctgggcggacagaagggagcggccgggtgcagctgcagcaacggccaccacttcctggggctcaggaccagcgtgctcgacttcagaggcctggagatgaagtgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 74 - DNA, 2428 bp, synthetic construct
  misc_feature 1..2428: note = Construct No. 018
  source 1..2428: mol_type = other DNA
aggcggggccggaggagggcaccagagaggctgctcaggagagagaaataatagaatatgtggtatagagtaaacatgagtgcggatgattgtggtatagagtaaagaattttgctgactaggacagaatattctttttagggtagaaatttagagtactatgagtgcggatagcctaaggaccactttaaatttgacacaatattgaaatttgaatggttttaacatttgaaggctgaaaaccaaaatactttgtagctaagtgttggaaacccgactcggccaataagtcgacagaccgtaaaataaggtcaatctaaactttatgataaatattcttgtttgatagcaatagcattgcaggaccaggacccaagggaagagaagatgccaaatcccatcgaggctaaagcaaaaacgatccaatttatgagcaaacccacactgaagtttcaaaattgttttctgaaaaaaaagtaaccagcaagttaaaaaatgagatggcgggaaagccaagtctcggttggtcgaggggttggttggggcgcagcctgacaagtgacaacggcagcaggatagtagcatcaggcgcaagccagcgcaggcggcagcgcgaggatttcgcttcacttagcggcaacggagacgctgcacccaaccaacacgagctccccctcacccgctgcgacgcgcgcgtcccacgagcggaagccccccgcgccgacgcgagcgcgggggctcgaccgaccgacccaacgcctccatctccaccgcgcgcaccaaatcgcactcccgtccgccccgccgatcgaacagccaccgctcacctctcccacccgccaaaaacctccggcctcctctcatattcatatagctagcccctgccacaaggtagagcgtcgctcacacctgcgtcgccctgcctcgcaatcgcgaatctgtcgagcacctgaggggtcggaggccgagagctagcctagcacgccggcctccgcgcgcgatggggcgatcgccgtgctgcgagaaggcgcacacgaacaagggcgcctggaccaaggaggaggacgaccgcctcgttgcctacatccgggcgcacggcgaggggtgctggcgctccctccccaaggccgcgggcctgctgcgctgcggcaagagctgccgcctgcgctggatcaactacctccgcccggacctcaagcgcggcaacttcaccgccgacgaggacgacctcatcgtcaagctccacagcctcctcggcaacaagtggtcgctcatcgccgcgcgcctccccggccgcaccgacaacgagatcaagaactactggaacacgcacatcaagcgcaagctcctcagccgcggcatcgaccccgtcacacaccgccccatcgccgacgcagccagaaacgtcaccatctccttccagcccgacgcgccgtcgcagcagcagctcagcgacgacgccgaggcgccgccgccgccgccgccgcagcagcagcagcagctcaagccgccgcccaggtgccccgacctcaatctcgacctctgcatcagcccgccctgccacaaggaagaagaggaccaggagctcgtcaagcccgccgccgtcaagcgcgagatgctgcaggccggccacggcactctaggactctgcttcggctgcagcctgggcctccagaagggcgccgccgggtgcacctgcagcagcaacagccacttcctggggctcagggtcggcatgctcctcgacttcagaggcctcgagatgaagtgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 75 - DNA, 2428 bp, synthetic construct
  misc_feature 1..2428: note = Construct No. 019
  source 1..2428: mol_type = other DNA
atactgaacattatgttgcataacatgtagataaggacacgaaaacatagaaagtttctcagttatatttacccatcaacatgaaataaaaaacaacaaagatgtcatagtgatgtttgtttcaacttaccaagggtgaccatgtcgtatttataataatattatatatttatatcgtcaatagaatattagtgttacggtgatattttagcacaccgattttttatatcatactgatgtttatcgttttgtatctatattttatatttgttttataataatattagatatttatttcgtcaatagaatattaatgttatgatgatactttactatattgattttacatatgatagtgatgttactccttccgtatctatattttatattagtttttatctcctggcaacacggtcacaacagaagagaagtttttcagaccgattccaggatcgattttttttttatatctgggctaagacatcaggtagagattgtttaacctttgcggctttccgcactgacggacccacccccaccgcatcaacggaacctaccaaccacccccgtgctccgaccccccatctgcccgtcttccaggttacgccccgcgcggccgcgcgcgcggaagctgtatcaccccacccgtcgacgtcgtcttcgcttcgaaaccccgcaaaaccccgcggaaaaaacccacctgctgcacgcacgcaccccctccctctccctccccatggcgcctcccctcacccaactctttgcttccattctttccatccacccgccaatgcgacgccgacgccgcaactccacccaccgcctgccagcgccacctcaccgcaccgcttccatcaccccgcgatcatgggctaccgctatatcaccacgcctccaacctccggcacgcttagcctctctctcccattctctcacacccaacacccagctatcacaccctgatccccgaggccgcgcgtcggggtgaggaggaggggccatggggcgatcgccgtgctgcgagaaggcgcacacgaacaagggcgcctggaccaaggaggaggacgaccgcctcgttgcctacatccgggcgcacggcgaggggtgctggcgctccctccccaaggccgcgggcctgctgcgctgcggcaagagctgccgcctgcgctggatcaactacctccgcccggacctcaagcgcggcaacttcaccgccgacgaggacgacctcatcgtcaagctccacagcctcctcggcaacaagtggtcgctcatcgccgcgcgcctccccggccgcaccgacaacgagatcaagaactactggaacacgcacatcaagcgcaagctcctcagccgcggcatcgaccccgtcacacaccgccccatcgccgacgcagccagaaacgtcaccatctccttccagcccgacgcgccgtcgcagcagcagctcagcgacgacgccgaggcgccgccgccgccgccgccgcagcagcagcagcagctcaagccgccgcccaggtgccccgacctcaatctcgacctctgcatcagcccgccctgccacaaggaagaagaggaccaggagctcgtcaagcccgccgccgtcaagcgcgagatgctgcaggccggccacggcactctaggactctgcttcggctgcagcctgggcctccagaagggcgccgccgggtgcacctgcagcagcaacagccacttcctggggctcagggtcggcatgctcctcgacttcagaggcctcgagatgaagtgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 76 - DNA, 2569 bp, synthetic construct
  misc_feature 1..2569: note = Construct No. 020
  source 1..2569: mol_type = other DNA
gagttaaattgatatggttttgtctgtcaaggtgccgtttctatggttggaggaggaaaatgaaattaggggaatagaacagtcgcaatccaaaagctattgttctctcttctacggtagttgatagttcgatacgtgtgtttgatgtgagagactgagaggagtgcacggtgcacctgccccgtaaggacgcggaactacattatcagaggctaggccggtactgttatacgcgacgttcacgaatcacgatgcgtaaaaaagtgaagcatgaaacagtactaggctctccacgggccacatcatgaaaaaactggtgcgacgccacgcttaacaatttgtcggtcgtaaactcgtaaagttaaaagccccacgacattgtcaaccaatatctcagcacatgaacttctctaacgagtacaacgaaaccgcatccgcaaaagcgccgtgaaccaaagctcttgccgtgctgtgccgtgccggtggccgtgaacatgtggaacacgaagaactcttgcgcgagatcggagcacctgacctcccacctcgcgtccggcccgtcgccgtcgcagcaagcgggctgtcaaaaacgacgccacagcgcgagcgctctcgccgatccggcggacccaccctcctcacctcgcgcaccaaccgcccgttcgctagtccgatcccccacccctcatcccccctacgccttgcaggttacgcgcctcgccgcggccaacgcaaaccaaaccaaatcccccgtcaccttcgcttcgaaaccccgcaaaaccccatggaagaaaacaccgaacacctgccgcgcgcacgcctcctcctcccccgcctctcctcctctctcccgttccatgtccgctcaaccttgcttccattctttccatccacccgccgatcgacgcgatgccgacgccccaaccccacccaccgcctgccagcgccaccccacctcgcgcctctgcggctatggctatatcaccatgcctccaacctccggtacgcttagcctctctctctctccccctcccattctgcgcattgctctctgcgcgcggtcgcgtgctgctgctcgcggcgccccggagcgtctcctttgggggagaggagaggagaggagaggagaggggggtgagccatggggcgatcgccgtgctgcgagaaggcgcacacgaacaagggcgcctggaccaaggaggaggacgaccgcctcgttgcctacatccgggcgcacggcgaggggtgctggcgctccctccccaaggccgcgggcctgctgcgctgcggcaagagctgccgcctgcgctggatcaactacctccgcccggacctcaagcgcggcaacttcaccgccgacgaggacgacctcatcgtcaagctccacagcctcctcggcaacaagtggtcgctcatcgccgcgcgcctccccggccgcaccgacaacgagatcaagaactactggaacacgcacatcaagcgcaagctcctcagccgcggcatcgaccccgtcacacaccgccccatcgccgacgcagccagaaacgtcaccatctccttccagcccgacgcgccgtcgcagcagcagctcagcgacgacgccgaggcgccgccgccgccgccgccgcagcagcagcagcagctcaagccgccgcccaggtgccccgacctcaatctcgacctctgcatcagcccgccctgccacaaggaagaagaggaccaggagctcgtcaagcccgccgccgtcaagcgcgagatgctgcaggccggccacggcactctaggactctgcttcggctgcagcctgggcctccagaagggcgccgccgggtgcacctgcagcagcaacagccacttcctggggctcagggtcggcatgctcctcgacttcagaggcctcgagatgaagtgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 77 - DNA, 2428 bp, synthetic construct
  misc_feature 1..2428: note = Construct No. 021
  source 1..2428: mol_type = other DNA
ttcaatgcaggatgacgccaagagggagaaaccaagcagaggtggacggtacaacgtgtaagtcaccgcaaaacgttgcagcttggatagtggccatcgagtggtgtgccgataccggcgcctgttctttacagcctcagctagtgttgttgtccgaggcaatttttccgacctattgtgttgctttcctctctgatagcttatggtaaaagatacaaagatgttgaggagtttgtacgccacttaattttgctcgtaacatacattgacaatcaagaggagccatggcattgcgatctgcttacacggcatattcttactggatggtgtacactacttaccctttttaatgcaagcatcaatccattgcttttctcactgcacacctgattcgtactgaaaacgtgaaacataaaaaaaaacaaaaatctagctgatgttggctctcggggcctcgagtctagtttgtcctagatggctaacctgatatgtgttggtcacgctcacgtttgaaccgagaaagagtgtgtgtgtgtgtgtgtcggcgtgctgctacaccagagcctccctgaatcgcaatgcgtgttaacgccagcatcgcaggatttcatctcacttgacaggttcagatggccttcctcctaccgtctgccatttatacacgcagtgacttaacgcttacacgagccggatggcccggatctcccccctgcaccatctcaccagaaaaacggtgaggcgtcaccgcaacccacccaccaaacacatccacgtcccttcaccgttggccttcgattttgcttcagctgcactacgacccctccaacacatttccctcgcgtctcgttgcgatctcaccttacgacgatctcgttccagcagcagcagcatcggcagcggcggcttgcttccgaagcgagcaatgcatggcgcgcgcggccgcgtgcgtgcgtgccttggcttgcgctctaatcaaaccgggacgccccaactcacggttatggggcgatcgccgtgctgcgagaaggcgcacacgaacaagggcgcctggaccaaggaggaggacgaccgcctcgttgcctacatccgggcgcacggcgaggggtgctggcgctccctccccaaggccgcgggcctgctgcgctgcggcaagagctgccgcctgcgctggatcaactacctccgcccggacctcaagcgcggcaacttcaccgccgacgaggacgacctcatcgtcaagctccacagcctcctcggcaacaagtggtcgctcatcgccgcgcgcctccccggccgcaccgacaacgagatcaagaactactggaacacgcacatcaagcgcaagctcctcagccgcggcatcgaccccgtcacacaccgccccatcgccgacgcagccagaaacgtcaccatctccttccagcccgacgcgccgtcgcagcagcagctcagcgacgacgccgaggcgccgccgccgccgccgccgcagcagcagcagcagctcaagccgccgcccaggtgccccgacctcaatctcgacctctgcatcagcccgccctgccacaaggaagaagaggaccaggagctcgtcaagcccgccgccgtcaagcgcgagatgctgcaggccggccacggcactctaggactctgcttcggctgcagcctgggcctccagaagggcgccgccgggtgcacctgcagcagcaacagccacttcctggggctcagggtcggcatgctcctcgacttcagaggcctcgagatgaagtgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 78 - DNA, 2275 bp, synthetic construct
  misc_feature 1..2275: note = Construct No. 022
  source 1..2275: mol_type = other DNA
aggcggggccggaggagggcaccagagaggctgctcaggagagagaaataatagaatatgtggtatagagtaaacatgagtgcggatgattgtggtatagagtaaagaattttgctgactaggacagaatattctttttagggtagaaatttagagtactatgagtgcggatagcctaaggaccactttaaatttgacacaatattgaaatttgaatggttttaacatttgaaggctgaaaaccaaaatactttgtagctaagtgttggaaacccgactcggccaataagtcgacagaccgtaaaataaggtcaatctaaactttatgataaatattcttgtttgatagcaatagcattgcaggaccaggacccaagggaagagaagatgccaaatcccatcgaggctaaagcaaaaacgatccaatttatgagcaaacccacactgaagtttcaaaattgttttctgaaaaaaaagtaaccagcaagttaaaaaatgagatggcgggaaagccaagtctcggttggtcgaggggttggttggggcgcagcctgacaagtgacaacggcagcaggatagtagcatcaggcgcaagccagcgcaggcggcagcgcgaggatttcgcttcacttagcggcaacggagacgctgcacccaaccaacacgagctccccctcacccgctgcgacgcgcgcgtcccacgagcggaagccccccgcgccgacgcgagcgcgggggctcgaccgaccgacccaacgcctccatctccaccgcgcgcaccaaatcgcactcccgtccgccccgccgatcgaacagccaccgctcacctctcccacccgccaaaaacctccggcctcctctcatattcatatagctagcccctgccacaaggtagagcgtcgctcacacctgcgtcgccctgcctcgcaatcgcgaatctgtcgagcacctgaggggtcggaggccgagagctagcctagcacgccggcctccgcgcgcgatggtacagccaaagaagaagtttcgtggagtcaggcagcggcactggggctcctgggtctctgagatcagacaccccctccttaaaaggagggtgtggctgggcacctttgagacggccgaggaggctgcgcgagcctacgatgaggctgctgtgctgatgagtggccgcaacgccaagaccaacttccccgtgcagaggaactccaccggtgatctcgccacggccgcagaccaggacgcccgtagcaatggcggtagcaggaactcctccgcgggcaacctgtcacagattctcagtgctaagctccgcaagtgctgcaaggcgccatctccgtccttaacctgcctccgcctcgaccccgagaagtcccacattggcgtgtggcaaaagcgcgcaggggcccgtgctgactccaactgggtgatgacggtggagctcaacaaagaggtagaaccaactgaacctgcagctcagcccacatcaacagcaacagcttcgcaagtgacaatggatgatgaggaaaagattgcgctgcaaatgatcgaggagttgctgagcaggagcagtccagcttcaccctcacatggagagggagagggtagctttgtcatctgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 79 - DNA, 2275 bp, synthetic construct
  misc_feature 1..2275: note = Construct No. 023
  source 1..2275: mol_type = other DNA
atactgaacattatgttgcataacatgtagataaggacacgaaaacatagaaagtttctcagttatatttacccatcaacatgaaataaaaaacaacaaagatgtcatagtgatgtttgtttcaacttaccaagggtgaccatgtcgtatttataataatattatatatttatatcgtcaatagaatattagtgttacggtgatattttagcacaccgattttttatatcatactgatgtttatcgttttgtatctatattttatatttgttttataataatattagatatttatttcgtcaatagaatattaatgttatgatgatactttactatattgattttacatatgatagtgatgttactccttccgtatctatattttatattagtttttatctcctggcaacacggtcacaacagaagagaagtttttcagaccgattccaggatcgattttttttttatatctgggctaagacatcaggtagagattgtttaacctttgcggctttccgcactgacggacccacccccaccgcatcaacggaacctaccaaccacccccgtgctccgaccccccatctgcccgtcttccaggttacgccccgcgcggccgcgcgcgcggaagctgtatcaccccacccgtcgacgtcgtcttcgcttcgaaaccccgcaaaaccccgcggaaaaaacccacctgctgcacgcacgcaccccctccctctccctccccatggcgcctcccctcacccaactctttgcttccattctttccatccacccgccaatgcgacgccgacgccgcaactccacccaccgcctgccagcgccacctcaccgcaccgcttccatcaccccgcgatcatgggctaccgctatatcaccacgcctccaacctccggcacgcttagcctctctctcccattctctcacacccaacacccagctatcacaccctgatccccgaggccgcgcgtcggggtgaggaggaggggccatggtacagccaaagaagaagtttcgtggagtcaggcagcggcactggggctcctgggtctctgagatcagacaccccctccttaaaaggagggtgtggctgggcacctttgagacggccgaggaggctgcgcgagcctacgatgaggctgctgtgctgatgagtggccgcaacgccaagaccaacttccccgtgcagaggaactccaccggtgatctcgccacggccgcagaccaggacgcccgtagcaatggcggtagcaggaactcctccgcgggcaacctgtcacagattctcagtgctaagctccgcaagtgctgcaaggcgccatctccgtccttaacctgcctccgcctcgaccccgagaagtcccacattggcgtgtggcaaaagcgcgcaggggcccgtgctgactccaactgggtgatgacggtggagctcaacaaagaggtagaaccaactgaacctgcagctcagcccacatcaacagcaacagcttcgcaagtgacaatggatgatgaggaaaagattgcgctgcaaatgatcgaggagttgctgagcaggagcagtccagcttcaccctcacatggagagggagagggtagctttgtcatctgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 80 - DNA, 2416 bp, synthetic construct
  misc_feature 1..2416: note = Construct No. 024
  source 1..2416: mol_type = other DNA
gagttaaattgatatggttttgtctgtcaaggtgccgtttctatggttggaggaggaaaatgaaattaggggaatagaacagtcgcaatccaaaagctattgttctctcttctacggtagttgatagttcgatacgtgtgtttgatgtgagagactgagaggagtgcacggtgcacctgccccgtaaggacgcggaactacattatcagaggctaggccggtactgttatacgcgacgttcacgaatcacgatgcgtaaaaaagtgaagcatgaaacagtactaggctctccacgggccacatcatgaaaaaactggtgcgacgccacgcttaacaatttgtcggtcgtaaactcgtaaagttaaaagccccacgacattgtcaaccaatatctcagcacatgaacttctctaacgagtacaacgaaaccgcatccgcaaaagcgccgtgaaccaaagctcttgccgtgctgtgccgtgccggtggccgtgaacatgtggaacacgaagaactcttgcgcgagatcggagcacctgacctcccacctcgcgtccggcccgtcgccgtcgcagcaagcgggctgtcaaaaacgacgccacagcgcgagcgctctcgccgatccggcggacccaccctcctcacctcgcgcaccaaccgcccgttcgctagtccgatcccccacccctcatcccccctacgccttgcaggttacgcgcctcgccgcggccaacgcaaaccaaaccaaatcccccgtcaccttcgcttcgaaaccccgcaaaaccccatggaagaaaacaccgaacacctgccgcgcgcacgcctcctcctcccccgcctctcctcctctctcccgttccatgtccgctcaaccttgcttccattctttccatccacccgccgatcgacgcgatgccgacgccccaaccccacccaccgcctgccagcgccaccccacctcgcgcctctgcggctatggctatatcaccatgcctccaacctccggtacgcttagcctctctctctctccccctcccattctgcgcattgctctctgcgcgcggtcgcgtgctgctgctcgcggcgccccggagcgtctcctttgggggagaggagaggagaggagaggagaggggggtgagccatggtacagccaaagaagaagtttcgtggagtcaggcagcggcactggggctcctgggtctctgagatcagacaccccctccttaaaaggagggtgtggctgggcacctttgagacggccgaggaggctgcgcgagcctacgatgaggctgctgtgctgatgagtggccgcaacgccaagaccaacttccccgtgcagaggaactccaccggtgatctcgccacggccgcagaccaggacgcccgtagcaatggcggtagcaggaactcctccgcgggcaacctgtcacagattctcagtgctaagctccgcaagtgctgcaaggcgccatctccgtccttaacctgcctccgcctcgaccccgagaagtcccacattggcgtgtggcaaaagcgcgcaggggcccgtgctgactccaactgggtgatgacggtggagctcaacaaagaggtagaaccaactgaacctgcagctcagcccacatcaacagcaacagcttcgcaagtgacaatggatgatgaggaaaagattgcgctgcaaatgatcgaggagttgctgagcaggagcagtccagcttcaccctcacatggagagggagagggtagctttgtcatctgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

SEQ ID NO: 81 - DNA, 2275 bp, synthetic construct
  misc_feature 1..2275: note = Construct No. 025
  source 1..2275: mol_type = other DNA
ttcaatgcaggatgacgccaagagggagaaaccaagcagaggtggacggtacaacgtgtaagtcaccgcaaaacgttgcagcttggatagtggccatcgagtggtgtgccgataccggcgcctgttctttacagcctcagctagtgttgttgtccgaggcaatttttccgacctattgtgttgctttcctctctgatagcttatggtaaaagatacaaagatgttgaggagtttgtacgccacttaattttgctcgtaacatacattgacaatcaagaggagccatggcattgcgatctgcttacacggcatattcttactggatggtgtacactacttaccctttttaatgcaagcatcaatccattgcttttctcactgcacacctgattcgtactgaaaacgtgaaacataaaaaaaaacaaaaatctagctgatgttggctctcggggcctcgagtctagtttgtcctagatggctaacctgatatgtgttggtcacgctcacgtttgaaccgagaaagagtgtgtgtgtgtgtgtgtcggcgtgctgctacaccagagcctccctgaatcgcaatgcgtgttaacgccagcatcgcaggatttcatctcacttgacaggttcagatggccttcctcctaccgtctgccatttatacacgcagtgacttaacgcttacacgagccggatggcccggatctcccccctgcaccatctcaccagaaaaacggtgaggcgtcaccgcaacccacccaccaaacacatccacgtcccttcaccgttggccttcgattttgcttcagctgcactacgacccctccaacacatttccctcgcgtctcgttgcgatctcaccttacgacgatctcgttccagcagcagcagcatcggcagcggcggcttgcttccgaagcgagcaatgcatggcgcgcgcggccgcgtgcgtgcgtgccttggcttgcgctctaatcaaaccgggacgccccaactcacggttatggtacagccaaagaagaagtttcgtggagtcaggcagcggcactggggctcctgggtctctgagatcagacaccccctccttaaaaggagggtgtggctgggcacctttgagacggccgaggaggctgcgcgagcctacgatgaggctgctgtgctgatgagtggccgcaacgccaagaccaacttccccgtgcagaggaactccaccggtgatctcgccacggccgcagaccaggacgcccgtagcaatggcggtagcaggaactcctccgcgggcaacctgtcacagattctcagtgctaagctccgcaagtgctgcaaggcgccatctccgtccttaacctgcctccgcctcgaccccgagaagtcccacattggcgtgtggcaaaagcgcgcaggggcccgtgctgactccaactgggtgatgacggtggagctcaacaaagaggtagaaccaactgaacctgcagctcagcccacatcaacagcaacagcttcgcaagtgacaatggatgatgaggaaaagattgcgctgcaaatgatcgaggagttgctgagcaggagcagtccagcttcaccctcacatggagagggagagggtagctttgtcatctgacacgtgtgaattacaggtgaccagctcgaatttccccgatagctttcgttcgtatcatcggtttcgacaacgttcgtcaagttcaatgcatcagtttcattgcgcacacaccagaatcctactgagttcgagtattatggcattgggaaacatgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaaatataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttg

(19) United States
(12) Patent Application Publication
(10) Pub. No.: US 2025/0122515 A1
(43) Pub. Date: Apr. 17, 2025

(54) COMPOSITIONS AND METHODS FOR INCREASING PLANT GROWTH AND IMPROVING MULTIPLE YIELD-RELATED TRAITS

(71) Applicant: AFINGEN, INC., Emeryville, CA (US)

(72) Inventor: Ai OIKAWA, Emeryville, CA (US)

(73) Assignee: AFINGEN, INC., Emeryville, CA (US)

(21) Appl. No.: 18/990,303

(22) Filed: Dec. 20, 2024

Related U.S. Application Data
(63) Continuation of application No. 18/172,699, filed on Feb. 22, 2023, now Pat. No. 12,203,084.
Application No. 18/172,699 is a continuation of application No. 16/965,809, filed on Jul. 29, 2020, now Pat. No. 11,613,760.
(60) Provisional application No. 62/623,279, filed on Jan. 29, 2018.

(51) Int. Cl. C12N 15/82; C07K 14/47

(52) U.S. Cl. CPC C12N 15/8223; C07K 14/47

(57) ABSTRACT

Compositions and methods for generating transgenic plants with increased plant growth and improved multiple yield-related traits, which comprise vascular xylem tissue-targeting overexpression of transcription factors (TFs) involved in vascular xylem cell development, are provided. One of the methods includes introducing into a plant cell a nucleic acid construct consisting of a heterologous plant tissue-specific promoter which is operably linked to a polynucleotide encoding a polypeptide of subgroup 4 R2R3-MYB transcription factor that shares at least 60% sequence similarity with SEQ ID NO: 04, and obtaining transformed plant cells; obtaining transgenic plants expressing the polypeptide from the transformed plant cells; and selecting a transgenic plant from the transgenic plants that expresses the polypeptide. Expression of the polypeptide in the selected transgenic plant exhibits both increased biomass yield and lower insoluble lignin content, as compared to a non-transgenic or wild-type control plant of the same plant species.

### CROSS-REFERENCE TO RELATED APPLICATIONS

This application is a continuation of U.S. application Ser. No. 18/172,699, filed Feb. 22, 2023, which is a continuation of U.S. application Ser. No. 16/965,809, filed Jul. 29, 2020, now U.S. Pat. No. 11,613,760, issued Mar. 28, 2023, which is the U.S. national phase of PCT Application No. PCT/US2019/015688, filed Jan. 29, 2019, which claims the benefit of U.S. Provisional Application No. 62/623,279, filed Jan. 29, 2018, the disclosures of which are incorporated in their entirety by reference herein.

### STATEMENT REGARDING FEDERALLY SPONSORED RESEARCH OR DEVELOPMENT

This invention was made with government support under Award Number 2016-33610-25368 awarded by the U.S. Department of Agriculture, Award Number NNX17CK04P awarded by the National Aeronautics and Space Administration, and Award Number DE-SC0011309 awarded by the U.S. Department of Energy. The government has certain rights in this invention.

### SEQUENCE LISTING

The xml file AALB0101PUSA1.xml of size 179 KB created Apr. 24, 2023, filed herewith, is hereby incorporated by reference.

### FIELD OF THE INVENTION

The present invention relates to genetic constructs and transgenic plants with vascular xylem tissue-targeting overexpression of transcription factors (TFs) involved in vascular xylem cell development, as well as their methods of use for enhancing plant growth and yield.

### BACKGROUND OF THE INVENTION

Yield is commonly defined as the measurable economic value of agricultural product from a crop. This may be defined in terms of quantity or quality, or a combination of both. Yield is directly dependent on several factors, for example, the number and size of the organs, plant architecture (i.e. number of tillers or branches), seed production, nutrient content, assimilation of metabolic precursors, root development, nutrient uptake, stress tolerance, and early vigor. Optimizing the above-mentioned factors may, therefore, contribute to increasing crop and horticultural yield. Depending on the end use, the modification of certain yield traits may be favored over others. For example, for applications such as forage or wood production, or as a biofuel resource, an increase in the vegetative parts of a plant may be desirable, and for applications such as flour, starch, or oil production, an increase in seed parameters may be particularly desirable. Such plant growth and/or yield-related traits may be improved by enhancing vascular tissue meristematic activity.

In higher plants, vascular tissues are important for transporting water and nutrients throughout the plant and providing physical support for upright growth. Primary constituents of the vascular tissues, xylem and phloem, are derived from the meristematic vascular procambium and cambium (Esau, Plant Anatomy (1965); Esau, Anatomy of Seed Plants (1977)). Xylem cells are particularly important for developing secondary cell walls that form the largest part of plant lignocellulosic biomass that consists of cellulose, hemicellulose, and lignin. Histochemical studies have indicated that lignification of the secondary cell wall generally occurs after the initial deposition of the cellulosic and hemicellulosic components and that it is initiated in a spatially distinct manner, beginning with the lignification of the middle lamella. Comparative studies of the patterns of secondary cell wall deposition in xylem cells in many different vascular plants have shown that the patterning of secondary cell wall deposition in these cells is a highly conserved process across species (Esau, Plant Anatomy (1965); Meylan and Butterfield, Three-Dimensional Structure of Wood (1972)).

Traditional molecular biology approaches using mutant analysis have identified a series of enzymes involved in the formation and accumulation of secondary cell wall mass (Turner and Somerville, “Collapsed Xylem Phenotype of Arabidopsis Identifies Mutants Deficient in Cellulose Deposition in the Secondary Cell Wall,” Plant Cell 9(5):689-701 (1997); Brown et al., “Identification of Novel Genes in Arabidopsis Involved in Secondary Cell Wall Formation Using Expression Profiling and Reverse Genetics,” Plant Cell 17(8):2281-95 (2005); Oikawa et al., “An Integrative Approach to the Identification of Arabidopsis and Rice Genes Involved in Xylan and Secondary Wall Development,” PLoS ONE 5(11):e15481 (2010); Hirano et al., “Survey of Genes Involved in Rice Secondary Cell Wall Formation Through a Co-Expression Network,” Plant Cell Physiol. 54(1):1803-21 (2013); Hao and Mohnen, “A Review of Xylan and Lignin Biosynthesis: Foundation for Studying Arabidopsis Irregular Xylem Mutants with Pleiotropic Phenotypes,” Crit. Rev. Biochem. Mol. Biol. 49(3):212-41 (2014)). Namely, irregular xylem (irx) mutants, which show collapsed xylem vessels in Arabidopsis, and brittle culm (bc) mutants, which show two-fold decrease in breaking strength compared with wild-type in rice, implicate these genes in secondary cell wall formation. The genes identified from the mutant screening were IRX1: AT4G18780 (AtCesA8), IRX2: AT5G49720 (AtKOR1), IRX3: AT5G17420 (AtCesA7), IRX4: AT1G15950 (AtCCR1), IRX5: AT5G44030 (AtCesA4), IRX6: AT5G15630 (AtCOBL4), IRX7: AT2G28110 (AtFRA8), IRX8: AT5G54690 (AtGAUT12), IRX9: AT2G37090 (glycosyltransferases family 43), IRX10: AT1G27440 (AtGUT1), IRX11: AT1G62990 (AtKNAT7), IRX12: AT2G38080 (AtLAC4), IRX13: At5G03170 (AtFLA11), IRX14: AT4G36890 (glycosyltransferases family 43), IRX15: AT3G50220 (DUF579), BC1: Os03g0416200 (OsCOBL5), BC2: (rice COBRA-like proteins), BC3: Os02g0738900 (OsDRP2B), BC6: Os09g0422500 (OsCESA9), BC7: Os01g0750300 (OsCESA4), BC10: Os05g0170000 (DUF266), BC11:0.0 g0750300 (CESA4), BC12: Os09g0114500 (OsKIN4A), BC14: Os02g0614100 (OsNST1), and BC15: Os09g0494200 (OsCTL1), which mainly consist of gene members coding endomembrane enzyme/protein for the cellulose and hemicellulose deposition and/or lignification. Integrated analysis of the series of mutants and co-expression gene datasets, in particular, revealed that distinct subgroups of CesA genes and proteins involved in cellulose biosynthesis in secondary cell walls are also conserved across plant species. For example, the products of three gene sets, such as the AtCesA4 (IRX5), AtCesA7 (IRX3), and AtCesA8 (IRX1) genes in Arabidopsis, the OsCesA4 (BC7), OsCesA7, and OsCesA9 (BC6) genes in rice, and the ZmCesA10, ZmCesAI1, and ZmCesA12 genes in maize, appear to function non-redundantly to catalyze cellulose biosynthesis in secondary cell walls. Xylan is the most abundant hemicellulose found in the secondary cell walls of plants and is thought to function as the major cellulose cross-linking component in secondary cell walls. Several Golgi-localized glycosyltransferases, including protein members from glycosyltransferases family 43 (i.e. the above-mentioned IRX9 and IRX14), have been involved in the biosynthesis of the xylose sugar backbone in developing xylem cells. Many other orthologous genes co-expressed with the cellulose synthase and xylan synthase genes in vascular xylem tissues are found in both dicot and monocot species, which implies common biological functions (Oikawa et al., “An Integrative Approach to the Identification of Arabidopsis and Rice Genes Involved in Xylan and Secondary Wall Development,” PLoS ONE 5(11):e15481 (2010)).

Since the expression amount of genes coding endomembrane enzyme/protein for the secondary cell wall cellulose/xylan deposition is specific and enormous in the vascular xylem tissues, their upstream sequence regions, namely promoters and 5′-UTR sequences, would be useful to overexpress heterogonous genes within the xylem tissues (Oikawa et al., “Golgi-Localized Enzyme Complexes for Plant Cell Wall Biosynthesis,” Trends Plant Sci. 18:49-58, (2013); Oikawa et al., “An Integrative Approach to the Identification of Arabidopsis and Rice Genes Involved in Xylan and Secondary Wall Development,” PLoS ONE 5(11):e15481 (2010)). As an example of the applications, the transgenic rice plants that expressed a sucrose synthase gene, OsSUS3, driven by the AtCesA8 (the above-mentioned IRX1) promoter maintained a normal growth with slightly increased biomass yields, and also reduced cellulose crystallinity and increased wall thickness, therefore leading to large improvements of both biomass saccharification and lodging (Fan et al., “AtCesA8-driven OsSUS3 Expression Leads to Largely Enhanced Biomass Saccharification and Lodging Resistance by Distinctively Altering Lignocellulose Features in Rice,” Biotechnol. Biofuels 10:221 (2017)). Another recent example of the successful tailoring of biomass properties is tissue-specific overexpression of master TF (Loque and Scheller, “Spatially Modified Gene Expression in Plants,” PCT Publication No. WO 2012/103555; Yang et al., “Engineering Secondary Cell Wall Deposition in Plants,” Plant Biotechnol. J. 11(3):325-35 (2013)). Tissue-specific overexpression of TF operably linked to a heterologous promoter that induces expression of a gene that is a downstream target of the TF enabled a positive feedback manner that regulates the amplified production of the secondary cell wall production in woody tissue. To accomplish this, a tissue-specific promoter sequence such as an IRX1, IRX3, IRX5, IRX8, IRX9, IRX14, IRX7, and IRX10 promoter was linked in with NAC-MYB TFs such as NST1, NST2, SND1/NST3, SND2, SND3, MYB103, MYB85, MYB46, MYB83, MYB58, or MYB63. This strategy is, however, limited to the accumulation of specific secondary cell wall compounds such as cellulose, xylan, and lignin in xylem and fiber tissues. Overall yield and/or yield-related traits have yet to be improved since the protocol uses the series of TFs only involved in secondary cell wall development, not in the upstream process such as vascular xylem cell differentiation and/or cell development.

R2R3-MYB subfamily 4 and ERF/AP2 subfamily B-6 have been well known as TFs that modulate secondary metabolites, increase wax content, and enhance biotic/abiotic stress tolerances. For the function of R2R3-MYB subfamily 4 and its biotechnology applications, see: Tak et al., “Overexpression of MusaMYB31, a R2R3 type MYB Transcription Factor Gene Indicate its Role as a Negative Regulator of Lignin Biosynthesis in Banana,” PLoS ONE 12(2):e0172695 (2017); Agarwal et al., “MYB31/MYB42 Syntelogs Exhibit Divergent Regulation of Phenylpropanoid Genes in Maize, Sorghum and Rice,” Sci. Rep. 6:28502 (2016); Poovaiah et al., “Sugarcane Transgenics Expression MYB Transcription Factors Show Improved Glucose Release,” Biotechnol Biofuels 9:143 (2016); Zhou et al., “Changing a Conserved Amino Acid in R2R3-MYB Transcription Repressors Results in Cytoplasmic Accumulation and Abolishes Their Repressive Activity in Arabidopsis,” Plant J. 84(2):395-403 (2015); Martin and Butelli, “Methods for Increasing the Anthocyanin Content of Citrus Fruit,” U.S. Patent Publication 20140007287; Rouster et al., “Production of Plants Having Improved Water-Deficit Tolerance,” U.S. Patent Publication 20130298282; Handakumbura and Hazen, “Transcriptional Regulation of Grass Secondary Cell Wall Biosynthesis: Playing Catch-Up With Arabidopsis Thaliana,” Front. Plant Sci. 3:74 (2012); Guan et al., “Methods of Modifying Lignin Biosynthesis and Improving Digestability,” U.S. Patent Publication 20120272406; Shen et al., “Compositions and Methods for Improved Plant Feedstock,” U.S. Patent Publication 20120322122; Shen et al., “Functional Characterization of the Switchgrass (Panicum virgatum) R2R3-MYB Transcription Factor PvMYB4 for Improvement of Lignocellulosic Feedstocks,” New Phytol. 193:121-36 (2012); Wang and Dixon, “On-Off Switches for Secondary Cell Wall Biosynthesis,” Mol. Plant. 5(2):297-303 (2012); Bedon et al., “Subgroup 4 R2R3-MYBs in Conifer Trees: Gene Family Expansion and Contribution to the Isoprenoid—and Flavonoid—Oriented Responses,” Journal of Experimental Botany 61(14):3847-3864 (2010); Fornale et al., “ZmMYB31 Directly Represses Maize Lignin Genes and Redirects the Phenylpropanoid Metabolic Flux,” Plant J. 64(4):633-44 (2010); Sonbol et al., “The Maize ZmMYB42 Represses the Phenylpropanoid Pathway and Affects the Cell Wall Structure, Composition and Degradability in Arabidopsis Thaliana,” Plant Mol. Biol. 70:283-96 (2009); Legay et al., “Molecular Characterization of EgMYB1, a Putative Transcriptional Repressor of the Lignin Biosynthetic Pathway,” Plant Sci. 173:542-9 (2007); Fornald et al., “Down-Regulation of the Maize and Arabidopsis thaliana Caffeic Acid O-methyl-transferase Genes by Two New Maize R2R3-MYB Transcription Factors,” Plant Mol. Biol. 62(6):809-23 (2006); Coraggio et al., “Use of the Myb4 Transcriptional Factor From Rice to Increase the Production of Secondary Metabolites by Transformed Plants,” PCT Publication No. WO 2005/080580; Preston et al., “AtMYB32 is Required for Normal Pollen Development in Arabidopsis thaliana,” Plant J. 40(6):979-95 (2004). For the function of ERF/AP2 subfamily B-6 and its biotechnology applications, see Xu et al., “Overexpression of the Transcription Factors GmSHN1 and GmSHN9 Differentially Regulates Wax and Cutin Biosynthesis, Alters Cuticle Properties, and Changes Leaf Phenotypes in Arabidopsis,” Int. J. Mol. Sci. 17(4):E587 (2016); Djemal and Khoudi, “Isolation and Molecular Characterization of a Novel WIN/SHN1 Ethylene-Responsive Transcription Factor TdSHN1 From Durum Wheat (Triticum turgidum L. subsp. durum) Protoplasma 252(6):1461-73 (2015); Al-Abdallat et al., “Over-Expression of SISHNI Gene Improves Drought Tolerance by Increasing Cuticular Wax Accumulation in Tomato,” Int. J. Mol. Sci. 15(11):19499-515 (2014); Sela et al., “Overexpression of AtSHN1/WIN1 provokes Unique Defense Responses,” PLoS One 8(7):e70146 (2013); Loque and Scheller, “Spatially Modified Gene Expression in Plants,” PCT Publication No. WO 2012/103555; Wang et al., “An Ethylene Response Factor OsWR1 Responsive to Drought Stress Transcriptionally Activate Wax Synthesis Related Genes and Increases Wax Production in Rice,” Plant Mol Biol. 78(3):275-88 (2012); Shi et al., “SHINE Transcription Factors Act Redundantly to Pattern the Archetypal Surface of Arabidopsis Flower Organs,” PLoS Genet. 7(5):e1001388 (2011); Kannangara et al., “The Transcription Factor WIN1/SHN1 Regulates Cutin Biosynthesis in Arabidopsis thaliana,” Plant Cell 19(4):1278-94 (2007); Aharoni et al., “The SHINE Clade of Transcription Factors and Their Use,” PCT Publication WO 2005/120215; Aharoni et al., “The SHINE clade of AP2 Domain Transcription Factors Activates Wax Biosynthesis, Alters Cuticle Properties, and Confers Drought Tolerance When Overexpressed in Arabidopsis,” Plant Cell 16(9):2463-80 (2004). Recent overexpression studies by traditional constitutive promoters demonstrated that a series of orthologous TFs from the R2R3-MYB subfamily 4 and ERF/AP2 subfamily B-6 are also potential regulators of the secondary cell wall NAC-MYB TFs (Hussey et al., “Navigating the Transcriptional Roadmap Regulating Plant Secondary Cell Wall Deposition,” Front. Plant Sci. 4:325 (2013); Yang and Wang, “Molecular Mechanisms for Vascular Development and Secondary Cell wall Formation,” Front. Plant Sci. 7:356 (2016); Ambavaram et al., “Coordinated Activation of Cellulose and Repression of Lignin Biosynthesis Pathways in Rice,” Plant Physiol. 155(2):916-31 (2011); Legay et al., “EgMYB1, an R2R3 MYB Transcription Factor from Eucalyptus Negatively Regulates Secondary Cell Wall Formation in Arabidopsis and Poplar,” New Phytol. 188(3):774-86 (2010)).

Although gene expression of Arabidopsis R2R3-MYB subfamily 4, MYB4, MYB7, and MYB32, are positively regulated by secondary wall MYB TF, MYB46, they are also shown to be involved in fine-tuning the upstream transcriptional regulation of developing vascular xylem cells. Overexpression of two maize homologs from the R2R3-MYB subfamily 4, namely ZmMYB31 and ZmMYB42, in Arabidopsis results in down-regulation of the lignin pathway and a patchy secondary cell wall deposition phenotype in fiber cells, which supports a repressive role for these proteins. In addition, there is molecular evidence showing that MYB4, MYB7, and MYB32 repress not only their own promoters but also the promoter of the secondary cell wall NAC master TF SND1/NST3 that regulates MYB46. Such negative regulations suggests that R2R3-MYB subfamily 4 may fine-tune the expressions and activities of secondary wall NAC-MYB-based transcriptional regulatory network for vascular xylem and fiber cells development.

The secondary cell wall master regulators in the endothecium of anthers include NST2, which is co-expressed with the SHN/WIN genes from ERF/AP2 subfamily B-6 and also with WRKY DNA-Binding Protein 12 (Wang et al., “Mutation of WRKY Transcription Factors Initiates Pith Secondary Wall Formation and Increases Stem Biomass in Dicotyledonous Plants,” Proc. Natl. Acad. Sci. U.S.A 107(51):22338-43 (2010); Yang et al., “PtrWRKY19, a Novel WRKY Transcription Factor, Contributes to the Regulation of Pith Secondary Wall Formation in Populus trichocarpa,” Sci. Rep. 6:18643 (2016)) that are believed to be upstream transcriptional regulators. The OsSHN1 gene, a homolog of Arabidopsis AtSHN2 in rice, is also tightly co-expressed with TFs and biosynthetic genes associated with the formation of the secondary cell wall in xylem and fiber cells. Although both AtSHN2 and OsSHN1 genes are suggested to regulate wax and lipid biosynthesis, they can also (a) enhance cellulose synthase genes expression; and (b) suppress lignin biosynthetic gene expression when they are overexpressed in rice. Additional molecular evidence shows that AtSHN2 can bind the promoters of secondary cell wall NAC-MYB TFs in rice, indicating an upstream mechanism of transcriptional regulation by SHN gene family in monocots.

The expression modulation of the TFs for the fundamental studies, however, has been controlled by constitutive promoters, which often show detrimental effects as yield drag. The previously noted vascular xylem tissue-targeting overexpression with the above-mentioned promoters has not been applied to the TFs that can potentially act before the secondary wall NAC-MYB TFs.

The present invention seeks to cure these deficiencies through the combination of promoters which preferably target vascular xylem tissue and a series of DNA transcription factors (TFs) involved in the transcriptional regulation of developing vascular xylem cells to enhance multiple yield-related traits in plants.

### SUMMARY

A first aspect of the present invention is directed to a nucleic acid construct that includes a polynucleotide encoding a transcription factor polypeptide and a heterologous, tissue-specific promoter operably linked to the polynucleotide encoding the transcription factor polypeptide, wherein the promoter specifically directs expression of the transcription factor polypeptide in vascular xylem tissue of a plant.

A second aspect of the present invention is directed to an expression vector that includes a nucleic acid construct of the present invention.

A third aspect of the present invention is directed to a recombinant host cell that includes a nucleic acid construct according to the first aspect of the invention or a recombinant expression vector according to the second aspect of the invention. In certain embodiments, the recombinant host cells are bacterial cells or plant cells.

A fourth aspect of the present invention is directed to a transgenic plant or transgenic plant seed that includes a nucleic acid construct according to the first aspect of the invention or a recombinant host cell according to the third aspect of the invention.

A fifth aspect of the present invention is directed to a plant having (i.e., including) a transgene that includes a heterologous, tissue-specific promoter operably linked to a polynucleotide encoding a transcription factor involved in vascular xylem cell development, wherein the promoter specifically directs expression of the transcription factor in vascular xylem tissue of the plant.

A sixth aspect of the present invention is directed to a rootstock, cutting, or seed obtained from a transgenic plant according to the fourth aspect of the invention or a plant according to the fifth aspect of the invention.

A seventh aspect of the invention is directed to a method of enhancing plant growth or yield by providing a transgenic plant or transgenic plant seed that is transformed with a nucleic acid construct according to the first aspect of the invention (which includes a transgenic plant or transgenic plant seed according to the fourth aspect of the invention). In one embodiment, the transgenic plant is provided, and then grown under conditions effective to permit the nucleic acid construct to express the transcription factor polypeptide in vascular xylem tissue of the transgenic plant, and thereby enhance plant growth or yield. In another embodiment, the transgenic plant seed is provided, planted in a growth medium, and a transgenic plant is then propagated from the transgenic plant seed to permit the nucleic acid construct to express the transcription factor polypeptide in vascular xylem tissue of the transgenic plant, and thereby enhance plant growth or yield.

An eighth aspect of the invention is directed to a method of enhancing plant growth or yield by providing a rootstock, cutting, or seed according to the sixth aspect of the invention, which rootstock, cutting, or seed is planted in a growth medium, and a transgenic plant is then propagated from the rootstock, cutting, or seed to permit the nucleic acid construct (or transgene) to express the transcription factor polypeptide in vascular xylem tissue of the transgenic plant, and thereby enhance plant growth or yield.

A ninth aspect of the invention is directed to a method of enhancing plant growth or yield by providing a plant according to the fifth aspect of the invention, and growing the plant under conditions effective to permit the transgene to express the transcription factor polypeptide in vascular xylem tissue of the transgenic plant, and thereby enhance plant growth or yield.

A tenth aspect of the present invention is directed to a method of planting, cultivating, or harvesting a part or all of a plant according to the first aspect of the invention or fifth aspect of the invention.

An eleventh aspect of the present invention is direct to a method of making a plant according to the fourth aspect of the invention or a plant according to the fifth aspect of the present invention. The method includes introducing a nucleic acid construct or transgene of the invention into a plant cell and propagating the plant from the plant cell.

A twelfth aspect of the present invention is directed to a method of enhancing degradability of plant biomass by providing a transgenic plant or transgenic plant seed that is transformed with a nucleic acid construct according to the first aspect of the invention (which includes a transgenic plant or transgenic plant seed according to the fourth aspect of the invention). In one embodiment, the transgenic plant is provided, and then grown under conditions effective to permit the nucleic acid construct to express the transcription factor polypeptide in vascular xylem tissue of the transgenic plant, and thereby enhance degradability of plant biomass. In another embodiment, the transgenic plant seed is provided, planted in a growth medium, and a transgenic plant is then propagated from the transgenic plant seed to permit the nucleic acid construct to express the transcription factor polypeptide in vascular xylem tissue of the transgenic plant, and thereby enhance degradability of plant biomass.

An thirteenth aspect of the invention is directed to a method of enhancing degradability of plant biomass by providing a rootstock, cutting, or seed according to the sixth aspect of the invention, which rootstock, cutting, or seed is planted in a growth medium, and a transgenic plant is then propagated from the rootstock, cutting, or seed to permit the nucleic acid construct (or transgene) to express the transcription factor polypeptide in vascular xylem tissue of the transgenic plant, and thereby enhance degradability of plant biomass.

A fourteenth aspect of the invention is directed to a method of enhancing degradability of plant biomass by providing a plant according to the fifth aspect of the invention, and growing the plant under conditions effective to permit the transgene to express the transcription factor polypeptide in vascular xylem tissue of the transgenic plant, and thereby enhance degradability of plant biomass.

The minimal cis-genic combination of (i) a promoter sequence that preferably targets vascular xylem tissues, and (ii) a TF polypeptides from an R2R3-MYB subfamily 4 or ERF/AP2 subfamily B-6 is unique. The accompanying Examples surprisingly demonstrate that multiple yield-related traits could be introduced into plant cells by the vascular xylem tissue-targeting overexpression of the TFs that are believed (a) to be upstream regulators of secondary cell wall NAC master TFs (i.e., SND1/NST3, NST1, NST2, VND6, VND7), and (b) to be involved in the vascular xylem cell development. The tissue-targeting manner of the TF overexpression also enables reduced lignin in only the vascular xylem tissue and maintains lignin in other tissue cells that are vital to the structural supports of the plant. This invention generated significantly improved crops with a combination of three beneficial traits: (1) accelerated root growth, (2) increased seeds/grains and vegetative biomass yields, and (3) enhanced degradability of inedible/lignocellulosic biomass. These traits may contribute to enhancing U.S. agricultural production, self-sustainability, the economy, food security, and bioenergy. The invention has wide applicability across plant species, including both monocots and dicots.

### BRIEF DESCRIPTION OF THE DRAWINGS

FIG. 1 illustrates the map and nucleotide sequence for construct 001, pAtCTL2-AtMYB32-tRBS, which includes the promoter and 5′ UTR from AtCTL2, the open reading frame of AtMYB32 (shaded in sequence), and the 3′ RBS transcription terminator.

FIG. 2 illustrates the map and nucleotide sequence for construct 002, pAtLAC4-AtMYB32-tRBS, which includes the promoter from AtLAC4, the open reading frame of AtMYB32 (shaded in sequence), and the 3′ RBS transcription terminator.

FIG. 3 illustrates the map and nucleotide sequence for construct 003, pAtCesA4-AtMYB32-tRBS, which includes the promoter from AtCesA4, the open reading frame of AtMYB32 (shaded in sequence), and the 3′ RBS transcription terminator.

FIG. 4 illustrates the map and nucleotide sequence for construct 004, pAtCesA8-AtMYB32-tRBS, which includes the promoter and 5′ UTR from AtCesA8, the open reading frame of AtMYB32 (shaded in sequence), and the 3′ RBS transcription terminator.

FIG. 5 illustrates the map and nucleotide sequence for construct 005, pAtFLA11-AtMYB32-tRBS, which includes the promoter and 5′ UTR from AtFLA11, the open reading frame of AtMYB32 (shaded in sequence), and the 3′ RBS transcription terminator.

FIG. 6 illustrates the map and nucleotide sequence for construct 006, pAtCesA7-AtMYB32-tRBS, which includes the promoter and 5′ UTR from AtCesA7, the open reading frame of AtMYB32 (shaded in sequence), and the 3′ RBS transcription terminator.

FIG. 7 illustrates the map and nucleotide sequence for construct 007, pAtIRX9-AtMYB32-tRBS, which includes the promoter and 5′ UTR from AtIRX9, the open reading frame of AtMYB32 (shaded in sequence), and the 3′ RBS transcription terminator.

FIG. 8 illustrates the map and nucleotide sequence for construct 008, pAtCesA4-AtMYB4-tRBS, which includes the promoter from AtCesA4, the open reading frame of AtMYB4 (shaded in sequence), and the 3′ RBS transcription terminator.

FIG. 9 illustrates the map and nucleotide sequence for construct 009, pAtCesA8-AtMYB4-tRBS, which includes the promoter and 5′ UTR from AtCesA8, the open reading frame of AtMYB4 (shaded in sequence), and the 3′ RBS transcription terminator.

FIG. 10 illustrates the map and nucleotide sequence for construct 010, pZmCesA12-ZmMYB31-tRBS, which includes the promoter from ZmCesA12, the open reading frame of ZmMYB31 (shaded in sequence), and the 3′ RBS transcription terminator.

FIG. 11 illustrates the map and nucleotide sequence for construct 011, pZmCesA11-ZmMYB31-tRBS, which includes the promoter from ZmCesA11, the open reading frame of ZmMYB31 (shaded in sequence), and the 3′ RBS transcription terminator.

FIG. 12 illustrates the map and nucleotide sequence for construct 012, pOsCesA4-ZmMYB31-tRBS, which includes the promoter from OsCesA4, the open reading frame of ZmMYB31 (shaded in sequence), and the 3′ RBS transcription terminator.

FIG. 13 illustrates the map and nucleotide sequence for construct 013, pOsCesA7-ZmMYB31-tRBS, which includes the promoter from OsCesA7, the open reading frame of ZmMYB31 (shaded in sequence), and the 3′ RBS transcription terminator.

FIG. 14 illustrates the map and nucleotide sequence for construct 014, pZmCesA12-ZmMYB42-tRBS, which includes the promoter from ZmCesA12, the open reading frame of ZmMYB42 (shaded in sequence), and the 3′ RBS transcription terminator.

FIG. 15 illustrates the map and nucleotide sequence for construct 015, pZmCesA11-ZmMYB42-tRBS, which includes the promoter from ZmCesA11, the open reading frame of ZmMYB42 (shaded in sequence), and the 3′ RBS transcription terminator.

FIG. 16 illustrates the map and nucleotide sequence for construct 016, pOsCesA4-ZmMYB42-tRBS, which includes the promoter from OsCesA4, the open reading frame of ZmMYB42 (shaded in sequence), and the 3′ RBS transcription terminator.

FIG. 17 illustrates the map and nucleotide sequence for construct 017, pOsCesA7-ZmMYB42-tRBS, which includes the promoter from OsCesA7, the open reading frame of ZmMYB42 (shaded in sequence), and the 3′ RBS transcription terminator.

FIG. 18 illustrates the map and nucleotide sequence for construct 018, pZmCesA12-PvMYB4-tRBS, which includes the promoter from ZmCesA12, the open reading frame of PvMYB4 (shaded in sequence), and the 3′ RBS transcription terminator.

FIG. 19 illustrates the map and nucleotide sequence for construct 019, pZmCesA11-PvMYB4-tRBS, which includes the promoter from ZmCesA11, the open reading frame of PvMYB4 (shaded in sequence), and the 3′ RBS transcription terminator.

FIG. 20 illustrates the map and nucleotide sequence for construct 020, pOsCesA4-PvMYB4-tRBS, which includes the promoter from OsCesA4, the open reading frame of PvMYB4 (shaded in sequence), and the 3′ RBS transcription terminator.

FIG. 21 illustrates the map and nucleotide sequence for construct 021, pOsCesA7-PvMYB4-tRBS, which includes the promoter from OsCesA7, the open reading frame of PvMYB4 (shaded in sequence), and the 3′ RBS transcription terminator.

FIG. 22 illustrates the map and nucleotide sequence for construct 022, pZmCesA12-OsSHN1-tRBS, which includes the promoter from ZmCesA12, the open reading frame of OsSHN1 (shaded in sequence), and the 3′ RBS transcription terminator.

FIG. 23 illustrates the map and nucleotide sequence for construct 023, pZmCesA11-OsSHN1-tRBS, which includes the promoter from ZmCesA11, the open reading frame of OsSHN1 (shaded in sequence), and the 3′ RBS transcription terminator.

FIG. 24 illustrates the map and nucleotide sequence for construct 024, pOsCesA4-OsSHN1-tRBS, which includes the promoter from OsCesA4, the open reading frame of OsSHN1 (shaded in sequence), and the 3′ RBS transcription terminator.

FIG. 25 illustrates the map and nucleotide sequence for construct 025, pOsCesA7-OsSHN1-tRBS, which includes the promoter from OsCesA7, the open reading frame of OsSHN1 (shaded in sequence), and the 3′ RBS transcription terminator.

FIG. 26 is a map of an exemplary plasmid operable in dicots, which includes construct 003 shown in FIG. 3. Similar dicot-functional plasmids containing constructs 001, 002 and 004-009 were also prepared.

FIG. 27 is a map of an exemplary plasmid operable in monocots, which includes construct 018 shown in FIG. 18. Similar monocot-functional plasmids containing constructs 010-017 and 019-025 were also prepared.

FIG. 28 is an image of representative control (empty vector) and vector-transformed alfalfa (Medicago sativa L. cv Regen S) using construct No. 003 (center) and construct 008 (right). The height difference between the control and transgenic plants is evident.

FIG. 29 is an image of representative control (empty vector) and vector-transformed canola (Brassica napus L. cv Westar) using construct No. 004. The height difference between the control and transgenic plants is evident.

FIG. 30 is an image of representative control (empty vector) and vector-transformed sorghum (Sorghum bicolor P898012) using construct No. 018. The height difference between the control and transgenic plant is evident.

FIG. 31 is an image of representative control (empty vector) and vector-transformed switchgrass (Panicum virgatum Alamo) using construct No. 018. The height difference between the control and transgenic plants is evident.

### DETAILED DESCRIPTION

The present invention is directed to recombinant nucleic acid constructs and transgenes as well as expression vectors and host cells useful for generating transgenic plants that preferentially express the transgenes in vascular xylem tissue of the plant. Transgenic plant parts are also encompassed by the present invention, as are various methods for making the transgenic plants and plant parts. Also encompassed by the present invention are methods that utilize the transgenic plants or plant parts, including methods for enhancing plant growth, enhancing plant yield, modifying plant lignin content, promoting earlier reproductive maturation, and enhancing degradability of plant biomass. These recombinant materials and their use in practicing the various methods are described below.

One aspect of the present invention is directed to a nucleic acid construct that includes a polynucleotide encoding a transcription factor (“TF”) polypeptide and a heterologous, tissue-specific promoter operably linked to the polynucleotide encoding the TF polypeptide, wherein the promoter specifically directs expression of the TF polypeptide in vascular xylem tissue of a plant.

According to one embodiment, the nucleic acid construct takes the form of a transgene that includes a heterologous, tissue-specific promoter operably linked to a polynucleotide encoding the TF polypeptide involved in vascular xylem cell development, and a 3′ transcription termination sequence that is operably linked to the polynucleotide encoding the TF, wherein the promoter specifically directs expression of the TF in vascular xylem tissue of the plant.

Thus, this invention involves the formation and use of synthetic oligonucleotides or nucleotide sequences. A synthetic sequence is one that is initially produced or reproduced in a laboratory setting. The structure of the synthetic sequence is altered or different from that found in the sequence that is directly isolated from its natural setting. A polynucleotide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety). “Operably linked” is intended to mean a functional linkage between two or more elements.

In these and other aspects of the invention, the TF polypeptide encoded by the nucleic acid construct, or transgene, is one that modulates expression of at least one gene, and possibly a series of genes (i.e., two or more), involved with cell wall and secondary metabolite biosynthetic pathways. Transcription factors are proteins that are involved in the process of transcribing DNA into RNA. Transcription factors have DNA-binding domains that allow them to bind to specific DNA sequences (e.g., promoter sequences, enhancer sequences, and silencers). In certain embodiment of the present invention, the TF polypeptide is a polypeptide that can act as an upstream transcriptional regulator to secondary wall master TFs as an upstream transcriptional regulator.

Suitable classes of TF polypeptides include, without limitation, an R2R3-MYB subfamily 4 TF polypeptide, an ERF/AP2 subfamily B-6 TF polypeptide, and combinations thereof (i.e., when co-expressed). Both R2R3-MYB subfamily 4 TFs and ERF/AP2 subfamily B-6 TFs are widely conserved among both monocots and dicots, and therefore it is contemplated that any of a variety of TFs from these classes can be utilized.

Non-limiting examples of both the R2R3-MYB subfamily 4 TF polypeptide and an ERF/AP2 subfamily B-6 TF polypeptide are provided in the examples, and include those listed below.

ERF/AP2 subfamily B-6 Transcription Factors: Arabidopsis AtSHN3 (SEQ ID NOS:1,37); Arabidopsis AtSHN1/WIN1 (SEQ ID NOS: 2.38); Arabidopsis AtSHN2 (SEQ ID NOS: 3.39); rice OsSHN1 (OsEREB19) (SEQ ID NOS: 7.43); rice OsSHN2 (OsEREB114) (SEQ ID NOS: 8.44); sorghum SbEREB63 (SEQ ID NOS: 13.49); sorghum SbEREB150 (SEQ ID NOS: 14.50); and maize ZmEREB46 (SEQ ID NOS: 17.53).

R2R3-MYB Subfamily 4 Transcription Factors: Arabidopsis AtMYB32 (SEQ ID NOS: 4.40); Arabidopsis AtMYB4 (SEQ ID NOS: 5.41); Arabidopsis MYB7 (SEQ ID NOS: 6.42); rice OsMYB108-L (SEQ ID NOS: 9.45); rice OsMYB108 (SEQ ID NOS: 10.46); poplar PdMYB221 (SEQ ID NOS: 11.47); poplar PdMYB156 (SEQ ID NOS: 12.48); sorghum SbMYB86 (SEQ ID NOS: 15.51); sorghum SbMYB23 (SEQ ID NOS: 16.52); maize ZmMYB42 (SEQ ID NOS: 18.54); maize ZmMYB31 (SEQ ID NOS: 19.55); and switchgrass PvMYB4 (SEQ ID NOS: 20.56).

As will be appreciated by persons of skill in the art, polynucleotides encoding homologous TFs can be isolated from other monocots and dicots. Such homologous TFs can be substantially similar to one another at the protein level, and polynucleotides encoding those TFs can be substantially identical at the nucleic acid level. “Substantially identical,” as used in the context of two nucleic acids or polypeptides, refers to a sequence that has at least 60% sequence identity with a reference sequence. Alternatively, percent identity can be any integer from 60% to 100% inclusive. In some embodiments, this identity is at least: 60%, 65%, 70%, 75%, 80%, 85%, 90%, 91%, 92%, 93%, 94%, 95%, 96%, 97%, 98%, or 99% compared to a reference sequence using the programs described herein; preferably BLAST using standard parameters, as described below. Embodiments of the present invention provide for nucleic acids encoding polypeptides that are substantially identical to any of the provided TFs sequences. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. The BLAST algorithm also performs a statistical analysis of the similarity between two sequences. One measure of similarity provided by the BLAST algorithm is the smallest sum probability, which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵ (0.00001), and most preferably less than about 10⁻¹⁰ (0.0000000001).

The polynucleotides encoding such TFs can also be used to isolate corresponding sequences from other plants. In this manner, methods such as PCR and the like can be used to identify such sequences based on their sequence homology to the sequences set forth herein. Sequences isolated based on their sequence identity to the entire sequences set forth herein or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 75%, 80%, 85%, 90%, 91%, 92%, 93%, 94%, 95%, 96%, 97%, 98%, 99%, or greater sequence identity. Functions of orthologs are often highly conserved among species. Thus, isolated polynucleotides that have transcription activation or enhancer activities, and which hybridize under stringent conditions to the sequences disclosed herein, or to variants or fragments thereof, are encompassed by the present invention.

Variant sequences may also be identified by analysis of existing databases of sequenced genomes. In this manner, corresponding TF or enhancer sequences can be identified and used in the methods of the invention.

As noted above, the nucleic acid construct, or transgene, or the present invention includes tissue-specific promoters that specifically direct expression of the TF polypeptide in vascular xylem tissue of a plant, fiber tissues of a plant, or both.

A promoter is a polynucleotide sequence capable of driving transcription of a coding sequence in a cell. Thus, promoters used in the polynucleotide constructs of the invention include cis-acting transcriptional control elements and regulatory sequences that are involved in regulating or modulating the timing and/or rate of transcription of a gene, in this case the nucleic acid construct, or transgene, that includes a coding sequence for a TF of the type described above. A plant promoter is a promoter capable of initiating transcription in plant cells. Whereas a constitutive promoter is one that is capable of initiating transcription in nearly all tissue types, a tissue-specific promoter initiates transcription in one or a few particular tissue types, and a cell type-specific promoter initiates transcription only in one or a few particular cell types. As used herein, “tissue-specific” does not preclude the promoter from causing initiation of transcription in multiple different types of plant tissues. Rather, the term “tissue-specific” is intended to connote that the promoter causes preferential expression in one or more, but not all, plant tissues. In preferred embodiments, the tissue-specific promoter induces a high level of expression in the one or more plant tissues or, alternatively, where the tissue-specific promoter induces a high level of expression in the one or more plant tissues, the expression level is preferably elevated in vascular xylem and fiber tissues.

Expression levels of a TF can be increased (e.g., by up-regulation or overexpression) relative to the expression level of TF in a wild-type or control plant. With respect to the promoters of the present invention, “specifically directs expression in vascular xylem and fiber tissues” or “vascular xylem tissue-targeting expression” means that the promoter causes expression of a TF of the present invention that is at least 3-fold (e.g., 5-fold, 10-fold, 20-fold, 50-fold, etc.) greater in at least a portion of the vascular xylem tissue of a plant compared to other cell types (e.g., compared to epidermal or mesophyll cells). Vascular xylem tissues of a plant include plant procambium/cambium, xylem, and fiber cell types. In some embodiments, specific expression in plant vascular xylem tissues can be limited to a portion of the vasculature, e.g., above ground (aerial), below ground (roots), cambium cells only, xylem cells only, or both cambium and xylem cells. Further, in certain embodiment of the present invention, the tissue-specific promoter directs expression of the TF polypeptide in aerial parts of the plant, in roots of the plant, or in both the aerial parts and the roots of the plant.

The tissue-specific promoter directs expression of the TF polypeptide involved in developmental process of vascular xylem tissue cells that occurs before secondary wall thickening progresses with polysaccharide deposition and lignification.

The expression level of the TF may be measured, for example, by assaying for the level of the TF in the plant. Measurement of TF levels can be carried out directly using any of a variety of protein assays (e.g., by Western Blot) or indirectly by measuring the level of RNA transcripts (e.g., by northern blot).

Classes of suitable tissue-specific promoter include gene promoters for secondary cell wall development, an endomembrane protein gene promoter, or a secondary wall cellulose synthase (CesA) promoter. Exemplary tissue-specific promoters that induce elevated expression in vascular xylem and/or fiber tissues include, without limitation, Arabidopsis AtCTL2 promoter (SEQ ID NO:21); Arabidopsis AtLAC4 promoter (SEQ ID NO:22); Arabidopsis AtCesA4 promoter (SEQ ID NO:23); Arabidopsis AtCesA8 promoter (SEQ ID NO:24); Arabidopsis AtFLA11 promoter (SEQ ID NO:25); Arabidopsis AtCesA7 promoter (SEQ ID NO:26); Arabidopsis AtIRX9 promoter (SEQ ID NO:27); rice OsFLA9 promoter (SEQ ID NO:28); rice OsCTL1 promoter (SEQ ID NO:29); rice OsCesA4 promoter (SEQ ID NO:30); rice OcCesA7 promoter (SEQ ID NO:31); rice OsLac10 promoter (SEQ ID NO:32); rice OsGT43J promoter (SEQ ID NO:33); maize ZmCesA10 promoter (SEQ ID NO:34); maize ZmCesA12 promoter (SEQ ID NO:35); maize ZmCesA11 promoter (SEQ ID NO:36).

As will be appreciated by persons of skill in the art, promoters from homologous genes can be isolated from other monocots and dicots. Such homologous promoters can be substantially identical at the nucleic acid level as defined above.

Alternative tissue-specific promoters that induce elevated expression in vascular xylem and/or fiber tissues can be identified by examining native protein expression levels in the specified plant tissues over the course of development. See Oikawa et al., “An Integrative Approach to the Identification of Arabidopsis and Rice Genes Involved in Xylan and Secondary Wall Development,” PLoS ONE 5(11):e15481 (2010), which is hereby incorporated by reference in its entirety.

As noted above, the nucleic acid construct, or transgenes, of the invention include 5′ and 3′ regulatory sequences operably linked to a TF polynucleotide.

As noted above, the nucleic acid construct, or transgene, also includes an operable 3′ regulatory region, selected from among those which are capable of providing correct transcription termination and polyadenylation of mRNA for expression in the host cell of choice, operably linked to a modified trait nucleic acid molecule of the present invention. A number of 3′ regulatory regions are known to be operable in plants, and any suitable 3′ regulatory region can be used in accordance with the present invention.

Exemplary 3′ regulatory regions include, without limitation, the nopaline synthase (“nos”) 3′ regulatory region (Fraley et al., “Expression of Bacterial Genes in Plant Cells,” Proc. Nat'l Acad. Sci. USA 80:4803-4807 (1983), which is hereby incorporated by reference in its entirety) and the cauliflower mosaic virus (“CaMV”) 3′ regulatory region (Odell et al., “Identification of DNA Sequences Required for Activity of the Cauliflower Mosaic Virus 35S Promoter,” Nature 313(6005):810-812 (1985), which is hereby incorporated by reference in its entirety); and the pea Ribulose-1,5-Bisphosphate carboxylase/oxygenase Small subunit E9 (“RBS” or “E9”) 3′ regulatory region (Coruzzi et al., “Tissue-specific and light-regulated expression of a pea nuclear gene encoding the small subunit of ribulose-1,5-bisphosphate carboxylase,” EMBO J. 3(8):1671-1679 (1984), which is hereby incorporated by reference in its entirety). Virtually any 3′ regulatory region known to be operable in plants would be suitable for use in conjunction with the present invention.

Further aspects of the present invention include expression vectors including the nucleic acid constructs, or transgenes, described herein, as well as host cells, transgenic plants (plant cells and plant seeds produced from such transgenic plants), and transgenic plant seeds or plant parts transformed with the nucleic acid constructs described herein.

The nucleotide sequences used in the present invention may be inserted into any of the many available expression vectors and cell systems using reagents that are well known in the art. Suitable vectors include, but are not limited to, the following viral vectors such as lambda vector system gt11, gt WES.tB, Charon 4, and plasmid vectors such as pG-Cha, p35S-Cha, pBR322, pBR325, pACYC177, pACYC1084, pUC8, pUC9, pUC18, pUC19, pLG339, pR290, pKC37, pKC101, SV 40, pBluescript II SK+/− or KS+/−(see “Stratagene Cloning Systems” Catalog (1993) from Stratagene, La Jolla, CA, which is hereby incorporated by reference in its entirety), pQE, pIH821, pGEX, pET series (see Studier et al., “Use of T7 RNA Polymerase to Direct Expression of Cloned Genes,” Gene Expression Technology vol. 185 (1990), which is hereby incorporated by reference in its entirety), and any derivatives thereof. Recombinant molecules can be introduced into cells via transformation, particularly transduction, conjugation, mobilization, or electroporation.

The DNA sequences are cloned into the vector using standard cloning procedures in the art, as described by Sambrook et al., Molecular Cloning: A Laboratory Manual, Second Edition, Cold Spring Harbor, NY: Cold Spring Harbor Press (1989), and Ausubel et al., Current Protocols in Molecular Biology, New York, N.Y: John Wiley & Sons (1989), which are hereby incorporated by reference in their entirety.

In preparing a nucleic acid construct for expression, the various nucleic acid sequences may normally be inserted or substituted into a bacterial plasmid. Any convenient plasmid may be employed, which will be characterized by having a bacterial replication system, a marker which allows for selection in a bacterium, and generally one or more unique, conveniently located restriction sites. Numerous plasmids, referred to as transformation vectors, are available for plant transformation. The selection of a vector will depend on the preferred transformation technique and target species for transformation. A variety of vectors are available for stable transformation using Agrobacterium tumefaciens, a soilborne bacterium that causes crown gall. Crown gall is characterized by tumors or galls that develop on the lower stem and main roots of the infected plant. These tumors are due to the transfer and incorporation of part of the bacterium plasmid DNA into the plant chromosomal DNA. This transfer DNA (T-DNA) is expressed along with the normal genes of the plant cell. The plasmid DNA, pTi, or Ti-DNA, for “tumor inducing plasmid,” contains the vir genes necessary for movement of the T-DNA into the plant. The T-DNA carries genes that encode proteins involved in the biosynthesis of plant regulatory factors, and bacterial nutrients (opines). The T-DNA is delimited by two 25 bp imperfect direct repeat sequences called the “border sequences.” By removing the oncogene and opine genes, and replacing them with a gene of interest, it is possible to transfer foreign DNA into the plant without the formation of tumors or the multiplication of Agrobacterium tumefaciens (Fraley et al., “Expression of Bacterial Genes in Plant Cells,” Proc. Nat'l Acad. Sci. 80:4803-4807 (1983), which is hereby incorporated by reference in its entirety).

Further improvement of this technique led to the development of the binary vector system (Bevan, “Binary Agrobacterium Vectors for Plant Transformation,” Nucleic Acids Res. 12:8711-8721 (1984), which is hereby incorporated by reference in its entirety). In this system, all the T-DNA sequences (including the borders) are removed from the pTi, and a second vector containing T-DNA is introduced into Agrobacterium tumefaciens. This second vector has the advantage of being replicable in E. coli as well as A. tumefaciens, and contains a multiclonal site that facilitates the cloning of a transgene. An example of a commonly-used vector is pBin19 (Frisch et al., “Complete Sequence of the Binary Vector Bin19,” Plant Mol. Biol. 27:405-409 (1995), which is hereby incorporated by reference in its entirety). Any appropriate vectors now known or later described for genetic transformation are suitable for use with the present invention.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

The different components described above can be ligated together to produce the expression systems which contain the nucleic acid constructs used in the present invention, using well known molecular cloning techniques as described in Sambrook et al., Molecular Cloning: A Laboratory Manual, Second Edition Cold Spring Harbor, NY: Cold Spring Harbor Press (1989), and Ausubel et al. Current Protocols in Molecular Biology, New York, N.Y: John Wiley & Sons (1989), which are hereby incorporated by reference in their entirety.

Once the nucleic acid construct has been prepared, it is ready to be incorporated into a host cell. Basically, this method is carried out by transforming a host cell with the nucleic acid construct under conditions effective to achieve transcription of the nucleic acid molecule in the host cell. This is achieved with standard cloning procedures known in the art, such as described by Sambrook et al., Molecular Cloning: A Laboratory Manual, Second Edition, Cold Spring Harbor Laboratory, Cold Spring Harbor, New York (1989), which is hereby incorporated by reference in its entirety. Suitable host cells are plant cells. Suitable host cells also include bacterial cells. Methods of transformation may result in transient or stable expression of the nucleic acid under control of the promoter. Stable transformation is intended to mean that the nucleotide construct introduced into a plant integrates into the genome of the plant and is capable of being inherited by the progeny thereof. Transient transformation is intended to mean that the nucleotide construct introduced into a plant is not stably integrated into the genome of the plant, but is maintained in the plant cell for a sufficient period of time to allow for the expression of the introduced genes. Preferably, the nucleic acid construct of the present invention is stably inserted into the genome of the recombinant plant cell as a result of the transformation, although transient expression can serve an important purpose, particularly when the plant under investigation is slow-growing.

Plant tissue suitable for transformation includes leaf tissue, root tissue, meristems, zygotic and somatic embryos, callus, protoplasts, tassels, pollen, embryos, anthers, and the like. The means of transformation chosen is that most suited to the tissue to be transformed.

Transient expression in plant tissue can be achieved by particle bombardment (Klein et al., “High-Velocity Microprojectiles for Delivering Nucleic Acids Into Living Cells,” Nature 327:70-73 (1987), which is hereby incorporated by reference in its entirety), also known as biolistic transformation of the host cell, as disclosed in U.S. Pat. Nos. 4,945,050; 5,036,006; and 5,100,792, all to Sanford et al., and in Emerschad et al., “Somatic Embryogenesis and Plant Development from Immature Zygotic Embryos of Seedless Grapes (Vitis vinifera),” Plant Cell Reports 14:6-12 (1995), which are hereby incorporated by reference in their entirety.

In particle bombardment, tungsten or gold microparticles (1 to 2 μm in diameter) are coated with the DNA of interest and then bombarded at the tissue using high pressure gas. In this way, it is possible to deliver foreign DNA into the nucleus and obtain a temporal expression of the gene under the current conditions of the tissue. Biologically active particles (e.g., dried bacterial cells containing the vector and heterologous DNA) can also be propelled into plant cells. Other variations of particle bombardment, now known or hereafter developed, can also be used.

An appropriate method of stably introducing the nucleic acid construct into plant cells is to infect a plant cell with Agrobacterium tumefaciens or Agrobacterium rhizogenes previously transformed with the nucleic acid construct of the present invention. As described supra, the Ti (or RI) plasmid of Agrobacterium enables the highly successful transfer of a foreign nucleic acid molecule into plant cells. A variation of Agrobacterium transformation uses vacuum infiltration in which whole plants are used (Senior, “Uses of Plant Gene Silencing,” Biotechnology and Genetic Engineering Reviews 15:79-119 (1998), which is hereby incorporated by reference in its entirety).

Yet another method of introduction is fusion of protoplasts with other entities, either minicells, cells, lysosomes, or other fusible lipid-surfaced bodies (Fraley et al., “Liposome-mediated Delivery of Tobacco Mosaic Virus RNA Into Tobacco Protoplasts: A Sensitive Assay for Monitoring Liposome-protoplast Interactions,” Proc. Natl. Acad. Sci. USA 79:1859-63 (1982), which is hereby incorporated by reference in its entirety). The nucleic acid molecule may also be introduced into the plant cells by electroporation (Fromm et al., “Expression of Genes Transferred into Monocot and Dicot Plant Cells by Electroporation,” Proc. Natl. Acad. Sci. USA 82:5824 (1985), which is hereby incorporated by reference in its entirety). In this technique, plant protoplasts are electroporated in the presence of plasmids containing the expression cassette. Electrical impulses of high field strength reversibly permeabilize biomembranes allowing the introduction of the plasmids. Electroporated plant protoplasts reform the cell wall, divide, and regenerate. Other methods of transformation include polyethylene-mediated plant transformation, micro-injection, physical abrasives, and laser beams (Senior, “Uses of Plant Gene Silencing,” Biotechnology and Genetic Engineering Reviews 15:79-119 (1998), which is hereby incorporated by reference in its entirety). The precise method of transformation is not critical to the practice of the present invention. Any method that results in efficient transformation of the host cell of choice is appropriate for practicing the present invention.

Yet a further method for introduction is by use of known techniques for genome editing or alteration. Such techniques for targeted genomic insertion involve, for example, inducing a double stranded DNA break precisely at one or more targeted genetic loci followed by integration of a chosen transgene or nucleic acid molecule (or construct) during repair. Such techniques or systems include, for example, zinc finger nucleases (“ZFNs”) (Urnov et al., “Genome Editing with Engineered Zinc Finger Nucleases,” Nat Rev Genet. 11: 636-646 (2010), which is hereby incorporated by reference in its entirety), transcription activator-like effector nucleases (“TALENs”) (Joung & Sander, “TALENs: A Widely Applicable Technology for Targeted Genome Editing,” Nat Rev Mol Cell Biol. 14: 49-55 (2013), which is hereby incorporated by reference in its entirety), clustered regularly interspaced short palindromic repeat (“CRISPR”)-associated endonucleases (e.g., CRISPR/CRISPR-associated (“Cas”) 9 systems) (Wiedenheft et al., “RNA-Guided Genetic Silencing Systems in Bacteria and Archaea,” Nat 482:331-338 (2012); Zhang et al., “Multiplex Genome Engineering Using CRISPR/Cas Systems,” Science 339(6121): 819-23 (2013); and Gaj et al., “ZFN, TALEN, and CRISPR/Cas-based Methods for Genome Engineering,” Cell 31(7):397-405 (2013), each of which is hereby incorporated by reference in its entirety).

In certain embodiments, transformation described herein is carried out by microinjection, Agrobacterium-mediated transformation, direct gene transfer, ballistic particle acceleration, whisker method transformation, vacuum infiltration, biolistic transformation, electroporation, micro-injection, polyethylene-mediated transformation, or laser-beam transformation.

After transformation, the transformed plant cells must be regenerated. Plant regeneration from cultured protoplasts is described in Evans et al., Handbook of Plant Cell Cultures, Vol. 1, New York, New York: MacMillan Publishing Co. (1983); Vasil, ed., Cell Culture and Somatic Cell Genetics of Plants, Vol. I (1984) and Vol. III (1986), Orlando: Acad. Press; and Fitch et al., “Somatic Embryogenesis and Plant Regeneration from Immature Zygotic Embryos of Papaya (Carica papaya L.),” Plant Cell Rep. 9:320 (1990), which are hereby incorporated by reference in their entirety.

Means for regeneration vary from species to species of plants, but generally a suspension of transformed protoplasts or a petri plate containing explants is first provided. Callus tissue is formed and shoots may be induced from callus and subsequently rooted. Alternatively, embryo formation can be induced in the callus tissue. These embryos germinate as natural embryos to form plants. The culture media will generally contain various amino acids and hormones, such as auxin and cytokinins. Efficient regeneration will depend on the medium, on the genotype, and on the history of the culture. If these three variables are controlled, then regeneration is usually reproducible and repeatable.

Preferably, transformed cells are first identified using a selection marker simultaneously introduced into the host cells along with the nucleic acid construct of the present invention. Suitable selection markers include, without limitation, markers encoding for antibiotic resistance, such as the neomycin phosphotransferae II (“nptII”) gene which confers kanamycin resistance (Fraley et al., “Expression of Bacterial Genes in Plant Cells,” Proc. Natl. Acad. Sci. USA 80:4803-4807 (1983), which is hereby incorporated by reference in its entirety), and the genes which confer resistance to gentamycin, G418, hygromycin, streptomycin, spectinomycin, tetracycline, chloramphenicol, and the like. Cells or tissues are grown on a selection medium containing the appropriate antibiotic, whereby generally only those transformants expressing the antibiotic resistance marker continue to grow. Other types of markers are also suitable for inclusion in the expression cassette of the present invention. For example, a gene encoding for herbicide tolerance, such as tolerance to sulfonylurea is useful, or the dhfr gene, which confers resistance to methotrexate (Bourouis et al., “Vectors Containing a Prokaryotic Dihydrofolate Reductase Gene Transform Drosophila Cells to Methotrexate-resistance,” EMBO J. 2:1099-1104 (1983), which is hereby incorporated by reference in its entirety). Similarly, “reporter genes,” which encode for enzymes providing for production of an identifiable compound are suitable. The most widely used reporter gene for gene fusion experiments has been uidA, a gene from Escherichia coli that encodes the β-glucuronidase protein, also known as GUS (Jefferson et al., “GUS Fusions: β Glucuronidase as a Sensitive and Versatile Gene Fusion Marker in Higher Plants,” EMBO J. 6:3901-3907 (1987), which is hereby incorporated by reference in its entirety). Similarly, enzymes providing for production of a compound identifiable by luminescence, such as luciferase, are useful. The selection marker employed will depend on the target species; for certain target species, different antibiotics, herbicide, or biosynthesis selection markers are preferred.

Plant cells and tissues selected by means of an inhibitory agent or other selection marker are then tested for the acquisition of the transgene (Sambrook et al., Molecular Cloning: A Laboratory Manual, Cold Spring Harbor, New York: Cold Spring Harbor Press (1989), which is hereby incorporated by reference in its entirety).

After a transgene containing a nucleic acid construct is stably incorporated in transgenic plants, the transgene can be transferred to other plants by sexual crossing. Any of a number of standard breeding techniques can be used, depending upon the species to be crossed. Once transgenic plants of this type are produced, the plants themselves can be transplanted to a suitable growth medium and cultivated in accordance with conventional procedure so that the nucleic acid construct is present in the resulting plants. Alternatively, transgenic seeds are recovered from the transgenic plants. These seeds can then be planted in a suitable growth medium and cultivated using conventional procedures to produce transgenic plants.

In these embodiments, suitable growth medium includes soil, soil-less particulate medium, or a liquid growth medium. Conditions for cultivating and harvesting may different depending on the type of growth medium and location, e.g., field, greenhouse, hydroponic environment, etc.

During subsequent growth and cultivation of the transgenic plants of the invention, it is also contemplated that individual plants may be selected based on their exhibiting one or more of the following properties: faster vegetative growth including that which leads to early maturation, increased biomass yields, enhanced root development, increased seed/grain production, improved nutrient contents in biomass, increased release of glucose saccharides, increased release of xylose saccharides, reduced lignin composition, and any combinations thereof.

The present invention may be used for transformation of any plant species, including, but not limited to, monocots and dicots. Examples of plant species of interest include, but are not limited to, the genus Abies, Acacia, Acer, Aegilops, Aesculus, Agave, Ailanthus, Alnus, Amborella, Amelanchier, Arabidopsis, Arbutus, Arctostaphylos, Artemisia, Asiminia, Asparagus, Atriplex, Atropa, Aucuba, Avena, Berberis, Betula, Brachypodium, Brassica, Buddleia, Buxus, Calocedrus, Calotropis, Camellia, Camptotheca, Campsis, Cannabis, Capsicum, Capsella, Carpinus, Carya, Castanea, Catalpa, Ceanothus, Cedrus, Celastrus, Celtis, Cephalanthus, Cercidium, Cercis, Chaenomeles, Chamaecyparis, Chilopsis, Chionanthus, Chrysothamnus, Cicer, Cistus, Citrus, Citrullus, Cladrastis, Clematis, Coleogynia, Cornus, Corylus, Cotinus, Cotoneaster, Cowania, Crataegus, Crataegus, Cucumis, Cupressus, Cytisus, Daphne, Daucus, Deutzia, Diospyros, Dioscorea, Elaeagnus, Ephedra, Erythranthe, Escallonia, Eucalyptus, Euonymus, Eutrema, Fagus, Forsythia, Fragaria, Fraxinus, Gaultheria, Gelsemium, Genlisea, Ginkgo, Gleditsia, Glycine, Grevillea, Gymnocladus, Gossypium, Hamamelis, Hebe, Helianthus, Heliamphora, Hibiscus, Heterocallis, Hordeum, Hydrangea, Hyoscyamus, Hypericum, Lactuca, Linum, Lolium, Lycopersicon, Ilex, Ipomea, Juglans, Juniperus, Kalmia, Kerria, Koelreuteria, Lagerstroemia, Larix, Larrea, Libocedrus, Ligustrum, Liquidambar, Liriodendron, Lonicera, Lotus, Maclura, Magnolia, Mahonia, Malus, Manihot, Majorana, Medicago, Menispermum, Morus, Myrica, Nicotiana, Nyssa, Oryza, Osmanthus, Ostrya, Oxydendron, Panicum, Pannesetum, Parthenocissus, Papaver, Persea, Phaseolus, Philadelphus, Photinia, Physocarpus, Picea, Pisum, Pinus, Pittosporum, Platanus, Populus, Podophyllum, Prosopis, Prunus, Pseudotsuga, Ptelea, Purshia, Pyrus, Quercus, Raphanus, Rhamnus, Rhaphiolepis, Rhododendron, Rhus, Ribes, Ricinus, Robinia, Rosa, Rubus, Salix, Sambucus, Sassafras, Sequoia, Secale, Setaria, Senecio, Shepherdia, Smilax, Sinapis, Solanum, Sophora, Sorbus, Sorghum, Spiraea, Staphylea, Stevia, Stewartia, Symphoricarpos, Syringa, Taxodium, Taxus, Theobroma, Thuja, Tilia, Triticum, Trigonella, Tsuga, Ulmus, Umbellularia, Vaccinium, Viburnum, Vitis, Vigna, Zanthoxylum, Zea, or Zelkova.

Further aspects of the invention relates to the planting, cultivating, or harvesting a part or all of a transgenic plant of the present invention.

In addition to transgenic plants, the present invention also relates to transgenic plant parts including plant seeds, rootstock, and cuttings removed from the transgenic plant (including both woody and herbaceous cuttings). In certain embodiments, the plant, plant seed, rootstock, or cutting is (or is from) a monocot, including but not limited to those identified above. In other embodiments, the plant, plant seed, rootstock, or cutting is (or is from) a dicot, including but not limited to those identified above.

The present invention is also directed to one or more methods of enhancing plant growth or plant yield. As used herein, “yield” is defined as the measurement of the amount of a crop that was harvested per unit of land area. Crop yield is the measurement often used for grains or cereals and is typically measured as the amount of plant harvested per unit area for a given time, i.e., metric tons per hectare or kilograms per hectare. Crop yield can also refer to the actual seed or biomass produced or generated by the plant. Thus, an “enhanced yield” refers to an increase in yield relative to a non-transgenic control plant. As used herein, “enhanced plant growth” encompasses a number of aspects including, without limitation, faster vegetative growth including that which leads to early maturation, increased biomass yields, enhanced root development, increased seed/grain production, improved nutrient contents in biomass, and any combinations thereof.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; or (d) the subject plant or plant cell itself, under conditions in which the gene of interest is not expressed. A “subject plant or plant cell” is one in which genetic alteration, such as transformation, has been effected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell.

According to one embodiment, this method is carried out by providing a transgenic plant transformed with a nucleic acid construct of the present invention and growing the plant under conditions effective to permit the nucleic acid construct to express the TF polypeptide in vascular xylem tissue of the transgenic plant, and thereby enhance plant growth or yield.

According to a second embodiment, this method is carried out by providing a transgenic plant seed transformed with a nucleic acid construct of the present invention, planting the transgenic plant seed in a growth medium, and propagating a transgenic plant from the transgenic plant seed to permit the nucleic acid construct to express the TF polypeptide in vascular xylem tissue of the transgenic plant, and thereby enhance plant growth or yield.

According to a third embodiment, this method is carried out by providing a rootstock, cutting, or seed from a transgenic plant of the present invention, introducing the rootstock, cutting, or seed into a growth medium; and propagating a transgenic plant from the rootstock, cutting, or seed to permit the nucleic acid construct to express the TF polypeptide in vascular xylem tissue of the transgenic plant, and thereby enhance plant growth or yield.

According to a fourth embodiment, this method is carried out by providing a plant comprising a transgene that includes a heterologous, tissue-specific promoter operably linked to a polynucleotide encoding a TF involved in vascular xylem cell development, wherein the promoter specifically directs expression of the TF in vascular xylem tissue of the plant, and growing the plant under conditions effective to permit the transgene to express the TF polypeptide in vascular xylem tissue of the transgenic plant, and thereby enhance plant growth or yield.

According to a fifth embodiment, this method is carried out by providing a rootstock, cutting, or seed obtained from a plant comprising a transgene that includes a heterologous, tissue-specific promoter operably linked to a polynucleotide encoding a TF involved in vascular xylem cell development, wherein the promoter specifically directs expression of the TF in vascular xylem tissue of the plant, introducing the rootstock, cutting, or seed into a growth medium, and propagating a plant from the rootstock, cutting, or seed to permit the transgene to express the TF polypeptide in vascular xylem tissue of the transgenic plant, and thereby enhance plant growth or yield.

The present invention is also directed to one or more methods of enhancing degradability of plant biomass. As used herein, enhanced degradability of plant biomass refers to the rate of biomass degradation when otherwise exposed to similar environmental conditions, using comparable amounts of plant biomass, as compared to the biomass of a control plant. Enhanced degradability may refer to any one or more of: (i) increased release of glucose saccharides, (ii) increased release of xylose saccharides, (iii) reduced lignin composition, and any combinations thereof.

According to one embodiment, this method is carried out by providing a transgenic plant of the present invention and growing the plant under conditions effective to permit the nucleic acid construct to express the TF polypeptide in vascular xylem tissue of the transgenic plant, and thereby enhance degradability of plant biomass.

According to a second embodiment, this method is carried out by providing a transgenic plant seed of the present invention, planting the transgenic plant seed in a growth medium, and propagating a transgenic plant from the transgenic plant seed to permit the nucleic acid construct to express the TF polypeptide in vascular xylem tissue of the transgenic plant, and thereby enhance degradability of plant biomass.

According to a third embodiment, this method is carried out by providing a rootstock, cutting, or seed of the present invention, introducing the rootstock, cutting, or seed into a growth medium, and propagating a transgenic plant from the rootstock, cutting, or seed to permit the nucleic acid construct to express the TF polypeptide in vascular xylem tissue of the transgenic plant, and thereby enhance degradability of plant biomass.

According to a fourth embodiment, this method is carried out by providing a plant of the present invention and growing the plant under conditions effective to permit the transgene to express the TF polypeptide in vascular xylem tissue of the transgenic plant, and thereby enhance degradability of plant biomass.

According to a fifth embodiment, this method is carried out by providing a rootstock, cutting, or seed of the present invention, introducing the rootstock, cutting, or seed into a growth medium, and propagating a plant from the rootstock, cutting, or seed to permit the transgene to express the TF polypeptide in vascular xylem tissue of the transgenic plant, and thereby enhance degradability of plant biomass.

### EXAMPLES

The examples below are intended to exemplify the practice of embodiments of the disclosure but are by no means intended to limit the scope thereof.

### Example 1—Gene Combinations of a Promoter and a TF

A series of simple gene cassettes comprising TFs driven by promoters active in the target tissues were generated (see Tables 1 and 2). The promoters in Table 2 were selected based on their expression profile corresponding to the development of xylem tissue (Oikawa et al., “An Integrative Approach to the Identification of Arabidopsis and Rice Genes Involved in Xylan and Secondary Wall Development,” PLoS ONE 5(11):e15481 (2010), which is hereby incorporated by reference in its entirety).

TABLE 1
Examples of Transcription Factors for Gene Combination

| Referenced | | | | | | | |
|---|---|---|---|---|---|---|---|
| expression | | | SEQ ID NO | | | | |
| database¹ | Expression² | Gene name | AA | NT | TF family | Species | Gene ID |
| Arabidopsis root | 291.76 | AtSHN3 | 1 | 37 | ERF/AP2 | Arabidopsis | At5g25390 |
| transcripts | | | | | subfamily B-6 | | |
| Arabidopsis root | 611.33 | AtSHN1/WIN1 | 2 | 38 | ERF/AP2 | Arabidopsis | At1g15360 |
| transcripts | | | | | subfamily B-6 | | |
| Arabidopsis root | N/A | AtSHN2 | 3 | 39 | ERF/AP2 | Arabidopsis | At5g11190 |
| transcripts | | | | | subfamily B-6 | | |
| Arabidopsis root | 448.22 | AtMYB32 | 4 | 40 | R2R3-MYB | Arabidopsis | At4g34990 |
| transcripts | | | | | Subfamily 4 | | |
| Arabidopsis root | 2052.79 | AtMYB4 | 5 | 41 | R2R3-MYB | Arabidopsis | At4g38620 |
| transcripts | | | | | Subfamily 4 | | |
| Arabidopsis root | 2630.65 | MYB7 | 6 | 42 | R2R3-MYB | Arabidopsis | At2g16720 |
| transcripts | | | | | Subfamily 4 | | |
| Rice mas | N/A | OsSHN1 | 7 | 43 | ERF/AP2 | Rice | LOC_Os02g10760/ |
| transcripts | | (OsEREB19) | | | subfamily B-6 | | Os02g0202000 |
| Rice mas | 5636.25 | OsSHN2 | 8 | 44 | ERF/AP2 | Rice | LOC_Os06g40150/ |
| transcripts | | (OsEREB114) | | | subfamily B-6 | | Os06g0604000 |
| Rice mas | N/A | OsMYB108-L | 9 | 45 | R2R3-MYB | Rice | Os08g0549000 |
| transcripts | | | | | Subfamily 4 | | |
| Rice mas | 3694.68 | OsMYB108 | 10 | 46 | R2R3-MYB | Rice | LOC_Os09g36730/ |
| transcripts | | | | | Subfamily 4 | | Os09g0538400 |
| Poplar | 206.76 | PdMYB221 | 11 | 47 | R2R3-MYB | Poplar | POPTR_0004s18020 |
| development | | | | | Subfamily 4 | | |
| transcripts | | | | | | | |
| Poplar | 3436.2 | PdMYB156 | 12 | 48 | R2R3-MYB | Poplar | POPTR_0009s13640 |
| development | | | | | Subfamily 4 | | |
| transcripts | | | | | | | |
| N/A | N/A | SbEREB63 | 13 | 49 | ERF/AP2 | Sorghum | Sb04g006970 |
| | | | | | subfamily B-6 | | |
| N/A | N/A | SbEREB150 | 14 | 50 | ERF/AP2 | Sorghum | Sb10g023600 |
| | | | | | subfamily B-6 | | |
| N/A | N/A | SbMYB86 | 15 | 51 | R2R3-MYB | Sorghum | Sb07g024890 |
| | | | | | Subfamily 4 | | |
| N/A | N/A | SbMYB23 | 16 | 52 | R2R3-MYB | Sorghum | Sb02g031190 |
| | | | | | Subfamily 4 | | |
| Maize leaf | 23.74 | ZmEREB46 | 17 | 53 | ERF/AP2 | Maize | GRMZM2G085678 |
| gradient | | | | | subfamily B-6 | | |
| transcripts | | | | | | | |
| Maize leaf | 12.97 | ZmMYB42 | 18 | 54 | R2R3-MYB | Maize | GRMZM2G419239 |
| gradient | | | | | Subfamily 4 | | |
| transcripts | | | | | | | |
| Maize leaf | 64.76 | ZmMYB31 | 19 | 55 | R2R3-MYB | Maize | GRMZM2G050305 |
| gradient | | | | | Subfamily 4 | | |
| transcripts | | | | | | | |
| N/A | N/A | PvMYB4 | 20 | 56 | R2R3-MYB | Switchgrass | Pavir. J16675.1 |
| | | | | | Subfamily 4 | | |

¹The Bio-Analytic Resource for Plant Biology, available online at http://bar.utoronto.ca/ and described in Toufighi et al, “The Botany Array Resource: e-Northerns, Expression Angling, and Promoter Analyses,” The Plant Journal 43: 153-63 (2005), each of which is hereby incorporated by reference in its entirety.
²Relative gene expression value in vascular tissues or xylem-related organ.

The sequences referenced in Table 1 are set forth below.

SEQ ID NO: 1
Met Val His Ser Lys Lys Phe Arg Gly Val Arg Gln Arg Gln Trp Gly
Ser Trp Val Ser Glu Ile Arg His Pro Leu Leu Lys Arg Arg Val Trp
Leu Gly Thr Phe Asp Thr Ala Glu Thr Ala Ala Arg Ala Tyr Asp Gln
Ala Ala Val Leu Met Asn Gly Gln Ser Ala Lys Thr Asn Phe Pro Val
Ile Lys Ser Asn Gly Ser Asn Ser Leu Glu Ile Asn Ser Ala Leu Arg
Ser Pro Lys Ser Leu Ser Glu Leu Leu Asn Ala Lys Leu Arg Lys Asn
Cys Lys Asp Gln Thr Pro Tyr Leu Thr Cys Leu Arg Leu Asp Asn Asp
Ser Ser His Ile Gly Val Trp Gln Lys Arg Ala Gly Ser Lys Thr Ser
Pro Asn Trp Val Lys Leu Val Glu Leu Gly Asp Lys Val Asn Ala Arg
Pro Gly Gly Asp Ile Glu Thr Asn Lys Met Lys Val Arg Asn Glu Asp
Val Gln Glu Asp Asp Gln Met Ala Met Gln Met Ile Glu Glu Leu Leu
Asn Trp Thr Cys Pro Gly Ser Gly Ser Ile Ala Gln Val
SEQ ID NO: 2
Met Val Gln Thr Lys Lys Phe Arg Gly Val Arg Gln Arg His Trp Gly
Ser Trp Val Ala Glu Ile Arg His Pro Leu Leu Lys Arg Arg Ile Trp
Leu Gly Thr Phe Glu Thr Ala Glu Glu Ala Ala Arg Ala Tyr Asp Glu
Ala Ala Val Leu Met Ser Gly Arg Asn Ala Lys Thr Asn Phe Pro Leu
Asn Asn Asn Asn Thr Gly Glu Thr Ser Glu Gly Lys Thr Asp Ile Ser
Ala Ser Ser Thr Met Ser Ser Ser Thr Ser Ser Ser Ser Leu Ser Ser
Ile Leu Ser Ala Lys Leu Arg Lys Cys Cys Lys Ser Pro Ser Pro Ser
Leu Thr Cys Leu Arg Leu Asp Thr Ala Ser Ser His Ile Gly Val Trp
Gln Lys Arg Ala Gly Ser Lys Ser Asp Ser Ser Trp Val Met Thr Val
Glu Leu Gly Pro Ala Ser Ser Ser Gln Glu Thr Thr Ser Lys Ala Ser
Gln Asp Ala Ile Leu Ala Pro Thr Thr Glu Val Glu Ile Gly Gly Ser
Arg Glu Glu Val Leu Asp Glu Glu Glu Lys Val Ala Leu Gln Met Ile
Glu Glu Leu Leu Asn Thr Asn
SEQ ID NO: 3
Met Val His Ser Arg Lys Phe Arg Gly Val Arg Gln Arg Gln Trp Gly
Ser Trp Val Ser Glu Ile Arg His Pro Leu Leu Lys Arg Arg Val Trp
Leu Gly Thr Phe Glu Thr Ala Glu Ala Ala Ala Arg Ala Tyr Asp Gln
Ala Ala Leu Leu Met Asn Gly Gln Asn Ala Lys Thr Asn Phe Pro Val
Val Lys Ser Glu Glu Gly Ser Asp His Val Lys Asp Val Asn Ser Pro
Leu Met Ser Pro Lys Ser Leu Ser Glu Leu Leu Asn Ala Lys Leu Arg
Lys Ser Cys Lys Asp Leu Thr Pro Ser Leu Thr Cys Leu Arg Leu Asp
Thr Asp Ser Ser His Ile Gly Val Trp Gln Lys Arg Ala Gly Ser Lys
Thr Ser Pro Thr Trp Val Met Arg Leu Glu Leu Gly Asn Val Val Asn
Glu Ser Ala Val Asp Leu Gly Leu Thr Thr Met Asn Lys Gln Asn Val
Glu Lys Glu Glu Glu Glu Glu Glu Ala Ile Ile Ser Asp Glu Asp Gln
Leu Ala Met Glu Met Ile Glu Glu Leu Leu Asn Trp Ser
SEQ ID NO: 4
Met Gly Arg Ser Pro Cys Cys Glu Lys Asp His Thr Asn Lys Gly Ala
Trp Thr Lys Glu Glu Asp Asp Lys Leu Ile Ser Tyr Ile Lys Ala His
Gly Glu Gly Cys Trp Arg Ser Leu Pro Arg Ser Ala Gly Leu Gln Arg
Cys Gly Lys Ser Cys Arg Leu Arg Trp Ile Asn Tyr Leu Arg Pro Asp
Leu Lys Arg Gly Asn Phe Thr Leu Glu Glu Asp Asp Leu Ile Ile Lys
Leu His Ser Leu Leu Gly Asn Lys Trp Ser Leu Ile Ala Thr Arg Leu
Pro Gly Arg Thr Asp Asn Glu Ile Lys Asn Tyr Trp Asn Thr His Val
Lys Arg Lys Leu Leu Arg Lys Gly Ile Asp Pro Ala Thr His Arg Pro
Ile Asn Glu Thr Lys Thr Ser Gln Asp Ser Ser Asp Ser Ser Lys Thr
Glu Asp Pro Leu Val Lys Ile Leu Ser Phe Gly Pro Gln Leu Glu Lys
Ile Ala Asn Phe Gly Asp Glu Arg Ile Gln Lys Arg Val Glu Tyr Ser
Val Val Glu Glu Arg Cys Leu Asp Leu Asn Leu Glu Leu Arg Ile Ser
Pro Pro Trp Gln Asp Lys Leu His Asp Glu Arg Asn Leu Arg Phe Gly
Arg Val Lys Tyr Arg Cys Ser Ala Cys Arg Phe Gly Phe Gly Asn Gly
Lys Glu Cys Ser Cys Asn Asn Val Lys Cys Gln Thr Glu Asp Ser Ser
Ser Ser Ser Tyr Ser Ser Thr Asp Ile Ser Ser Ser Ile Gly Tyr Asp
Phe Leu Gly Leu Asn Asn Thr Arg Val Leu Asp Phe Ser Thr Leu Glu
Met Lys
SEQ ID NO: 5
Met Gly Arg Ser Pro Cys Cys Glu Lys Ala His Thr Asn Lys Gly Ala
Trp Thr Lys Glu Glu Asp Glu Arg Leu Val Ala Tyr Ile Lys Ala His
Gly Glu Gly Cys Trp Arg Ser Leu Pro Lys Ala Ala Gly Leu Leu Arg
Cys Gly Lys Ser Cys Arg Leu Arg Trp Ile Asn Tyr Leu Arg Pro Asp
Leu Lys Arg Gly Asn Phe Thr Glu Glu Glu Asp Glu Leu Ile Ile Lys
Leu His Ser Leu Leu Gly Asn Lys Trp Ser Leu Ile Ala Gly Arg Leu
Pro Gly Arg Thr Asp Asn Glu Ile Lys Asn Tyr Trp Asn Thr His Ile
Arg Arg Lys Leu Ile Asn Arg Gly Ile Asp Pro Thr Ser His Arg Pro
Ile Gln Glu Ser Ser Ala Ser Gln Asp Ser Lys Pro Thr Gln Leu Glu
Pro Val Thr Ser Asn Thr Ile Asn Ile Ser Phe Thr Ser Ala Pro Lys
Val Glu Thr Phe His Glu Ser Ile Ser Phe Pro Gly Lys Ser Glu Lys
Ile Ser Met Leu Thr Phe Lys Glu Glu Lys Asp Glu Cys Pro Val Gln
Glu Lys Phe Pro Asp Leu Asn Leu Glu Leu Arg Ile Ser Leu Pro Asp
Asp Val Asp Arg Leu Gln Gly His Gly Lys Ser Thr Thr Pro Arg Cys
Phe Lys Cys Ser Leu Gly Met Ile Asn Gly Met Glu Cys Arg Cys Gly
Arg Met Arg Cys Asp Val Val Gly Gly Ser Ser Lys Gly Ser Asp Met
Ser Asn Gly Phe Asp Phe Leu Gly Leu Ala Lys Lys Glu Thr Thr Ser
Leu Leu Gly Phe Arg Ser Leu Glu Met Lys
SEQ ID NO: 6
Met Gly Arg Ser Pro Cys Cys Glu Lys Glu His Met Asn Lys Gly Ala
Trp Thr Lys Glu Glu Asp Glu Arg Leu Val Ser Tyr Ile Lys Ser His
Gly Glu Gly Cys Trp Arg Ser Leu Pro Arg Ala Ala Gly Leu Leu Arg
Cys Gly Lys Ser Cys Arg Leu Arg Trp Ile Asn Tyr Leu Arg Pro Asp
Leu Lys Arg Gly Asn Phe Thr His Asp Glu Asp Glu Leu Ile Ile Lys
Leu His Ser Leu Leu Gly Asn Lys Trp Ser Leu Ile Ala Ala Arg Leu
Pro Gly Arg Thr Asp Asn Glu Ile Lys Asn Tyr Trp Asn Thr His Ile
Lys Arg Lys Leu Leu Ser Lys Gly Ile Asp Pro Ala Thr His Arg Gly
Ile Asn Glu Ala Lys Ile Ser Asp Leu Lys Lys Thr Lys Asp Gln Ile
Val Lys Asp Val Ser Phe Val Thr Lys Phe Glu Glu Thr Asp Lys Ser
Gly Asp Gln Lys Gln Asn Lys Tyr Ile Arg Asn Gly Leu Val Cys Lys
Glu Glu Arg Val Val Val Glu Glu Lys Ile Gly Pro Asp Leu Asn Leu
Glu Leu Arg Ile Ser Pro Pro Trp Gln Asn Gln Arg Glu Ile Ser Thr
Cys Thr Ala Ser Arg Phe Tyr Met Glu Asn Asp Met Glu Cys Ser Ser
Glu Thr Val Lys Cys Gln Thr Glu Asn Ser Ser Ser Ile Ser Tyr Ser
Ser Ile Asp Ile Ser Ser Ser Asn Val Gly Tyr Asp Phe Leu Gly Leu
Lys Thr Arg Ile Leu Asp Phe Arg Ser Leu Glu Met Lys
SEQ ID NO: 7
Met Gly Gln Ser Lys Lys Lys Phe Arg Gly Val Arg Gln Arg His Trp
Gly Ser Trp Val Ser Glu Ile Arg His Pro Leu Leu Lys Arg Arg Val
Trp Leu Gly Thr Phe Glu Thr Ala Glu Glu Ala Ala Arg Ala Tyr Asp
Glu Ala Ala Ile Leu Met Ser Gly Arg Asn Ala Lys Thr Asn Phe Pro
Val Ala Arg Asn Ala Thr Gly Glu Leu Thr Pro Ala Ala Ala Val Ala
Gly Arg Asp Gly Arg Val Gly Gly Gly Ser Gly Ser Ser Ser Ser Met
Thr Ala Asn Gly Gly Gly Asn Ser Leu Ser Gln Ile Leu Ser Ala Lys
Leu Arg Lys Cys Cys Lys Thr Pro Ser Pro Ser Leu Thr Cys Leu Arg
Leu Asp Pro Glu Lys Ser His Ile Gly Val Trp Gln Lys Arg Ala Gly
Ala Arg Ala Asp Ser Ser Trp Val Met Thr Val Glu Leu Asn Lys Asp
Thr Ala Val Ser Ser Ala Ala Thr Val Ala Ala Ala Thr Ala Val Ser
Ser Ser Asp Gln Pro Thr Pro Ser Asp Ser Thr Val Thr Thr Thr Ser
Thr Ser Thr Thr Gly Ser Pro Ser Pro Pro Pro Pro Ala Met Asp Asp
Glu Glu Arg Ile Ala Leu Gln Met Ile Glu Glu Leu Leu Gly Arg Ser
Gly Pro Gly Ser Pro Ser His Gly Leu Leu His Gly Gly Glu Gly Ser
Leu Val Ile
SEQ ID NO: 8
Met Gly Gln Ser Lys Lys Lys Phe Arg Gly Val Arg Gln Arg His Trp
Gly Ser Trp Val Ser Glu Ile Arg His Pro Leu Leu Lys Arg Arg Val
Trp Leu Gly Thr Phe Glu Thr Ala Glu Glu Ala Ala Arg Ala Tyr Asp
Glu Ala Ala Ile Leu Met Ser Gly Arg Asn Ala Lys Thr Asn Phe Pro
Val Ala Arg Asn Ala Thr Gly Glu Leu Thr Pro Ala Ala Ala Val Ala
Gly Arg Asp Gly Arg Val Gly Gly Gly Ser Gly Ser Ser Ser Ser Met
Thr Ala Asn Gly Gly Gly Asn Ser Leu Ser Gln Ile Leu Ser Ala Lys
Leu Arg Lys Cys Cys Lys Thr Pro Ser Pro Ser Leu Thr Cys Leu Arg
Leu Asp Pro Glu Lys Ser His Ile Gly Val Trp Gln Lys Arg Ala Gly
Ala Arg Ala Asp Ser Ser Trp Val Met Thr Val Glu Leu Asn Lys Asp
Thr Ala Val Ser Ser Ala Ala Thr Val Ala Ala Ala Thr Ala Val Ser
Ser Ser Asp Gln Pro Thr Pro Ser Asp Ser Thr Val Thr Thr Thr Ser
Thr Ser Thr Thr Gly Ser Pro Ser Pro Pro Pro Pro Ala Met Asp Asp
Glu Glu Arg Ile Ala Leu Gln Met Ile Glu Glu Leu Leu Gly Arg Ser
Gly Pro Gly Ser Pro Ser His Gly Leu Leu His Gly Gly Glu Gly Ser
Leu Val Ile
SEQ ID NO: 9
Met Gly Arg Ser Pro Cys Cys Glu Lys Glu His Thr Asn Lys Gly Ala
Trp Thr Lys Glu Glu Asp Glu Arg Leu Val Ala Tyr Ile Arg Ala His
Gly Glu Gly Cys Trp Arg Ser Leu Pro Lys Ala Ala Gly Leu Leu Arg
Cys Gly Lys Ser Cys Arg Leu Arg Trp Ile Asn Tyr Leu Arg Pro Asp
Leu Lys Arg Gly Asn Phe Thr Ala Asp Glu Asp Asp Leu Ile Ile Lys
Leu His Ser Leu Leu Gly Asn Lys Trp Ser Leu Ile Ala Ala Arg Leu
Pro Gly Arg Thr Asp Asn Glu Ile Lys Asn Tyr Trp Asn Thr His Ile
Arg Arg Lys Leu Leu Gly Arg Gly Ile Asp Pro Val Thr His Arg Pro
Val Asn Ala Ala Ala Ala Thr Ile Ser Phe His Pro Gln Pro Pro Pro
Thr Thr Lys Glu Glu Gln Leu Ile Leu Ser Lys Pro Pro Lys Cys Pro
Asp Leu Asn Leu Asp Leu Cys Ile Ser Pro Pro Ser Cys Gln Glu Glu
Asp Asp Asp Tyr Glu Ala Lys Pro Ala Met Ile Val Arg Ala Pro Glu
Leu Gln Arg Arg Arg Gly Gly Leu Cys Phe Gly Cys Ser Leu Gly Leu
Gln Lys Glu Cys Lys Cys Ser Gly Gly Gly Ala Gly Ala Gly Ala Gly
Asn Asn Phe Leu Gly Leu Arg Ala Gly Met Leu Asp Phe Arg Ser Leu
Pro Met Lys
SEQ ID NO: 10
Met Gly Arg Ser Pro Cys Cys Glu Lys Ala His Thr Asn Lys Gly Ala
Trp Thr Lys Glu Glu Asp Asp Arg Leu Ile Ala Tyr Ile Lys Ala His
Gly Glu Gly Cys Trp Arg Ser Leu Pro Lys Ala Ala Gly Leu Leu Arg
Cys Gly Lys Ser Cys Arg Leu Arg Trp Ile Asn Tyr Leu Arg Pro Asp
Leu Lys Arg Gly Asn Phe Thr Glu Glu Glu Asp Glu Leu Ile Ile Lys
Leu His Ser Leu Leu Gly Asn Lys Trp Ser Leu Ile Ala Gly Arg Leu
Pro Gly Arg Thr Asp Asn Glu Ile Lys Asn Tyr Trp Asn Thr His Ile
Arg Arg Lys Leu Leu Ser Arg Gly Ile Asp Pro Val Thr His Arg Pro
Ile Asn Asp Ser Ala Ser Asn Ile Thr Ile Ser Phe Glu Ala Ala Ala
Ala Ala Ala Arg Asp Asp Lys Ala Ala Val Phe Arg Arg Glu Asp His
Pro His Gln Pro Lys Ala Val Thr Val Ala Gln Glu Gln Gln Ala Ala
Ala Asp Trp Gly His Gly Lys Pro Leu Lys Cys Pro Asp Leu Asn Leu
Asp Leu Cys Ile Ser Leu Pro Ser Gln Glu Glu Pro Met Met Met Lys
Pro Val Lys Arg Glu Thr Gly Val Cys Phe Ser Cys Ser Leu Gly Leu
Pro Lys Ser Thr Asp Cys Lys Cys Ser Ser Phe Leu Gly Leu Arg Thr
Ala Met Leu Asp Phe Arg Ser Leu Glu Met Lys
SEQ ID NO: 11
Met Gly Arg Ser Pro Cys Cys Glu Lys Ala His Thr Asn Lys Gly Ala
Trp Thr Lys Glu Glu Asp Asp Arg Leu Ile Ala Tyr Ile Arg Thr His
Gly Glu Gly Cys Trp Arg Ser Leu Pro Lys Ala Ala Gly Leu Leu Arg
Cys Gly Lys Ser Cys Arg Leu Arg Trp Ile Asn Tyr Leu Arg Pro Asp
Leu Lys Arg Gly Asn Phe Thr Glu Glu Glu Asp Glu Leu Ile Ile Lys
Leu His Ser Leu Leu Gly Asn Lys Trp Ser Leu Ile Ala Gly Arg Leu
Pro Gly Arg Thr Asp Asn Glu Ile Lys Asn Tyr Trp Asn Thr His Ile
Arg Arg Lys Leu Leu Asn Arg Gly Ile Asp Pro Ala Thr His Arg Pro
Leu Asn Glu Pro Ala Gln Glu Ala Ser Thr Thr Ile Ser Phe Ser Thr
Thr Thr Ser Val Lys Glu Glu Ser Leu Ser Ser Val Lys Glu Glu Ser
Asn Lys Glu Lys Ile Ile Ser Ala Ala Ala Phe Ile Cys Lys Glu Glu
Lys Thr Pro Val Gln Glu Arg Cys Pro Asp Leu Asn Leu Glu Leu Arg
Ile Ser Leu Pro Cys Gln Asn Gln Pro Asp Arg His Gln Ala Phe Lys
Thr Gly Gly Ser Thr Ser Leu Cys Phe Ala Cys Ser Leu Gly Leu Gln
Asn Ser Lys Asp Cys Ser Cys Ser Val Ile Val Gly Thr Ile Gly Ser
Ser Ser Ser Ala Gly Ser Lys Thr Gly Tyr Asp Phe Leu Gly Met Lys
Ser Gly Val Leu Asp Tyr Arg Gly Leu Glu Met Lys
SEQ ID NO: 12
Met Gly Arg Ser Pro Cys Cys Glu Lys Ala His Thr Asn Lys Gly Ala
Trp Thr Lys Glu Glu Asp Asp Arg Leu Val Ala Tyr Ile Arg Ala His
Gly Glu Gly Cys Trp Arg Ser Leu Pro Lys Ala Ala Gly Leu Leu Arg
Cys Gly Lys Ser Cys Arg Leu Arg Trp Ile Asn Tyr Leu Arg Pro Asp
Leu Lys Arg Gly Asn Phe Thr Glu Ala Glu Asp Glu Leu Ile Ile Lys
Leu His Ser Leu Leu Gly Asn Lys Trp Ser Leu Ile Ala Gly Arg Leu
Pro Gly Arg Thr Asp Asn Glu Ile Lys Asn Tyr Trp Asn Thr His Ile
Arg Arg Lys Leu Leu Asn Arg Gly Ile Asp Pro Ala Thr His Arg Pro
Leu Asn Glu Pro Ala Val Gln Glu Ala Thr Thr Thr Ile Ser Phe Thr
Thr Thr Thr Thr Ser Val Leu Glu Glu Glu Ser Leu Gly Ser Ile Ile
Lys Glu Glu Asn Lys Glu Lys Ile Ile Ser Ala Thr Ala Phe Val Cys
Lys Glu Glu Lys Thr Gln Val Gln Glu Arg Cys Pro Asp Leu Asn Leu
Glu Leu Gly Ile Ser Leu Pro Ser Gln Asn Gln Pro Asp His His Gln
Pro Phe Lys Thr Gly Gly Ser Arg Ser Leu Cys Phe Ala Cys Ser Leu
Gly Leu Gln Asn Ser Lys Asp Cys Ser Cys Asn Val Ile Val Ser Thr
Val Gly Ser Ser Gly Ser Thr Ser Thr Lys Thr Gly Tyr Asp Phe Leu
Gly Met Lys Ser Gly Val Leu Asp Tyr Arg Ser Leu Glu Met Lys
SEQ ID NO: 13
Met Pro Thr Pro Thr Pro Thr Pro Thr Pro Thr Cys Gly Asp Gly Ser
Leu Ala Gly Phe Ala Leu Leu Leu Arg Gly Glu Lys Arg Val Ala Asn
Gly Ala Arg Gly Gly Arg Gly Ile Gly Gly Glu Arg Ala Lys Ile Ile
Arg Arg Arg His Ala Glu Lys Thr His Gly Arg Arg Glu Arg Gly Gly
His Arg Arg Ser His Arg Leu Ala Tyr Pro Leu Trp Val Leu Asp Ile
Arg Ser Pro Asn Gly Ile Met Leu Gly Ile Phe Arg Gly Ala Ala Leu
Trp Leu Trp Thr Leu Ala Trp His Met
SEQ ID NO: 14
Ser Lys Lys Lys Phe Arg Gly Val Arg Gln Arg His Trp Met Val Gln
Gly Ser Trp Val Ser Glu Ile Arg His Pro Leu Leu Lys Arg Arg Val
Trp Leu Gly Thr Phe Glu Thr Ala Glu Glu Ala Ala Arg Ala Tyr Asp
Glu Ala Ala Val Leu Met Ser Gly Arg Asn Ala Lys Thr Asn Phe Pro
Val Pro Arg Thr Ala Thr Gly Glu Leu Ala Pro Val Pro Ala Ala Arg
Asp Ala Arg Gly Gly Gly Gly Ser Ser Ser Ala Ala Ala Ala Pro Gly
Gly Gly Thr Ser Asn Leu Ser Gln Ile Leu Ser Ala Lys Leu Arg Lys
Cys Cys Lys Thr Pro Ser Pro Ser Leu Thr Cys Leu Arg Leu Asp Pro
Glu Lys Ser His Ile Gly Val Trp Gln Lys Arg Ala Gly Ala Arg Ala
Asp Ser Ser Trp Val Met Thr Val Gln Leu Asn Lys Asp Val Pro Pro
Pro Ala Ser Ser Ser Gly Glu Glu Pro Val Pro Ser Asp Gly Gly Ala
Ala Ala Thr Thr Pro Thr Ser Thr Ser Thr Ser Ser Thr Val Thr Thr
Thr Gly Ser Pro Pro Pro Ala Met Met Met Asp Asp Glu Glu Arg Ile
Ala Leu Gln Met Ile Glu Glu Leu Leu Gly Ser Ser His Ser His Gly
Met Phe Gln Gly Ala Ala Gly Ser Ile Val Ile
SEQ ID NO: 15
Met Gly Arg Ser Pro Cys Cys Glu Lys Ala His Thr Asn Lys Gly Ala
Trp Thr Lys Glu Glu Asp Asp Arg Leu Val Ala Tyr Ile Arg Ala His
Gly Glu Gly Cys Trp Arg Ser Leu Pro Lys Ala Ala Gly Leu Met Arg
Cys Gly Lys Ser Cys Arg Leu Arg Trp Ile Asn Tyr Leu Arg Pro Asp
Leu Lys Arg Gly Asn Phe Thr Ala Asp Glu Asp Asp Leu Ile Ile Lys
Leu His Ser Leu Leu Gly Asn Lys Trp Ser Leu Ile Ala Ala Arg Leu
Pro Gly Arg Thr Asp Asn Glu Ile Lys Asn Tyr Trp Asn Thr His Ile
Arg Arg Lys Leu Leu Gly Arg Gly Ile Asp Pro Val Thr His Arg Pro
Ile Ala Asp Ala Gly Ala Gly Thr Val Thr Thr Ile Ser Phe Gln Pro
Asn Lys Pro Asn Ala Ala Val Ala Ala Gln Ala Pro Gln His Gln Pro
Ile Lys Ala Val Ala Thr Ala Val Val Lys Val Pro Arg Cys Pro Asp
Leu Asn Leu Asp Leu Cys Ile Ser Pro Pro Cys Gln Gln Lys Glu Asp
Glu Glu Leu Asp Leu Lys Pro Ala Val Val Val Lys Arg Glu Val Leu
Gln Ala Gly His Gly Gly Ser Leu Cys Phe Gly Cys Ser Leu Gly Ile
Gln Lys Gly Ala Pro Gly Cys Ser Cys Ser Ser Ser Asn Ser His His
Arg Phe Leu Gly Leu Arg Ser Gly Met Leu Asp Phe Arg Gly Leu Glu
Met Lys
SEQ ID NO: 16
Met Gly Arg Ser Pro Cys Cys Glu Lys Ala His Thr Asn Lys Gly Ala
Trp Thr Lys Glu Glu Asp Asp Arg Leu Val Ala Tyr Ile Lys Ala His
Gly Glu Gly Cys Trp Arg Ser Leu Pro Lys Ala Ala Gly Leu Leu Arg
Cys Gly Lys Ser Cys Arg Leu Arg Trp Ile Asn Tyr Leu Arg Pro Asp
Leu Lys Arg Gly Asn Phe Thr Glu Glu Glu Asp Glu Leu Ile Ile Lys
Leu His Ser Leu Leu Gly Asn Lys Trp Ser Leu Ile Ala Gly Arg Leu
Pro Gly Arg Thr Asp Asn Glu Ile Lys Asn Tyr Trp Asn Thr His Ile
Arg Arg Lys Leu Leu Ser Arg Gly Ile Asp Pro Val Thr His Arg Pro
Ile Asn Glu His Thr Ser Asn Ile Thr Ile Ser Phe Glu Ala Ala Ala
Ala Ala Arg Asp Arg Glu Glu Asn Lys Gly Ala Val Phe Arg Leu Glu
Glu His Asn Lys Ala Thr Ala Ala Ala Ala Ala Ala Ile Gly Arg Asp
His His Gln Asn His His Pro Ala Gly Asp Trp Gly Gln Gly Lys Pro
Leu Lys Cys Pro Asp Leu Asn Leu Asp Leu Cys Ile Ser Pro Pro Ala
Ala Pro Cys Gln Glu Glu Lys Ala Met Val Thr Met Lys Pro Val Lys
Arg Glu Ala Gly Leu Cys Phe Ser Cys Ser Leu Gly Leu Pro Lys Ser
Ala Asp Cys Lys Cys Ser Asn Phe Leu Gly Leu Arg Thr Ala Met Leu
Asp Phe Arg Ser Leu Glu Met Lys
SEQ ID NO: 17
Met Thr Glu Asn Leu His Ser Arg Lys Met Val Gln Pro Lys Lys Phe
Arg Gly Val Arg Gln Arg His Trp Gly Ser Trp Val Ser Glu Ile Arg
His Pro Leu Leu Lys Arg Arg Val Trp Leu Gly Thr Phe Glu Thr Ala
Glu Glu Ala Ala Arg Ala Tyr Asp Glu Ala Ala Val Leu Met Ser Gly
Arg Asn Ala Lys Thr Asn Phe Pro Ile Gln Arg Ser Ser Thr Gly Glu
Pro Thr Pro Ala Ala Gly Arg Asp Ala Arg Ser Asn Phe Ser Ser Gly
Ser Ser Thr Thr Asn Leu Ser Gln Ile Leu Ser Ala Lys Leu Arg Lys
Cys Cys Lys Ala Pro Ser Pro Ser Leu Thr Cys Leu Arg Leu Asp Pro
Glu Lys Ser His Ile Gly Val Trp Gln Lys Arg Ala Gly Ala Arg Ala
Asp Ser Asn Trp Val Met Thr Val Glu Leu Asn Lys Asp Ala Ala Ser
Thr Asp Ala Ala Ser Gln Ser Thr Ser Ala Thr Thr Ala Pro Pro Ala
Thr Pro Met Asp Glu Glu Glu Arg Ile Ala Leu Gln Met Ile Glu Glu
Leu Leu Ser Ser Ser Ser Pro Ala Ser Pro Ser Asn Gly Asp Asp Gln
Gly Arg Phe Ile Ile
SEQ ID NO: 18
Met Gly Arg Ser Pro Cys Cys Glu Lys Ala His Thr Asn Arg Gly Ala
Trp Thr Lys Glu Glu Asp Glu Arg Leu Val Ala Tyr Val Arg Ala His
Gly Glu Gly Cys Trp Arg Ser Leu Pro Arg Ala Ala Gly Leu Leu Arg
Cys Gly Lys Ser Cys Arg Leu Arg Trp Ile Asn Tyr Leu Arg Pro Asp
Leu Lys Arg Gly Asn Phe Thr Ala Asp Glu Asp Asp Leu Ile Val Lys
Leu His Ser Leu Leu Gly Asn Lys Trp Ser Leu Ile Ala Ala Arg Leu
Pro Gly Arg Thr Asp Asn Glu Ile Lys Asn Tyr Trp Asn Thr His Ile
Arg Arg Lys Leu Leu Gly Ser Gly Ile Asp Pro Val Thr His Arg Arg
Val Ala Gly Gly Ala Ala Thr Thr Ile Ser Phe Gln Pro Ser Pro Asn
Thr Ala Val Ala Ala Ala Ala Glu Thr Ala Ala Gln Ala Pro Ile Lys
Ala Glu Glu Thr Ala Ala Val Lys Ala Pro Arg Cys Pro Asp Leu Asn
Leu Asp Leu Cys Ile Ser Pro Pro Cys Gln His Glu Asp Asp Gly Glu
Glu Glu Glu Glu Glu Leu Asp Leu Ile Lys Pro Ala Val Val Lys Arg
Glu Ala Leu Gln Ala Gly His Gly His Gly His Gly Leu Cys Leu Gly
Cys Gly Leu Gly Gly Gln Lys Gly Ala Ala Gly Cys Ser Cys Ser Asn
Gly His His Phe Leu Gly Leu Arg Thr Ser Val Leu Asp Phe Arg Gly
Leu
SEQ ID NO: 19
Met Gly Arg Ser Pro Cys Cys Glu Lys Ala His Thr Asn Lys Gly Ala
Trp Thr Lys Glu Glu Asp Glu Arg Leu Val Ala His Ile Arg Ala His
Gly Glu Gly Cys Trp Arg Ser Leu Pro Lys Ala Ala Gly Leu Leu Arg
Cys Gly Lys Ser Cys Arg Leu Arg Trp Ile Asn Tyr Leu Arg Pro Asp
Leu Lys Arg Gly Asn Phe Thr Glu Glu Glu Asp Glu Leu Ile Val Lys
Leu His Ser Val Leu Gly Asn Lys Trp Ser Leu Ile Ala Gly Arg Leu
Pro Gly Arg Thr Asp Asn Glu Ile Lys Asn Tyr Trp Asn Thr His Ile
Arg Arg Lys Leu Leu Ser Arg Gly Ile Asp Pro Val Thr His Arg Pro
Val Thr Glu His His Ala Ser Asn Ile Thr Ile Ser Phe Glu Thr Glu
Val Ala Ala Ala Ala Arg Asp Asp Lys Lys Gly Ala Val Phe Arg Leu
Glu Glu Glu Glu Glu Arg Asn Lys Ala Thr Met Val Val Gly Arg Asp
Arg Gln Ser Gln Ser Gln Ser His Ser His Pro Ala Gly Glu Trp Gly
Gln Gly Lys Arg Pro Leu Lys Cys Pro Asp Leu Asn Leu Asp Leu Cys
Ile Ser Pro Pro Cys Gln Glu Glu Glu Glu Met Glu Glu Ala Ala Met
Arg Val Arg Pro Ala Val Lys Arg Glu Ala Gly Leu Cys Phe Gly Cys
Ser Leu Gly Leu Pro Arg Thr Ala Asp Cys Lys Cys Ser Ser Ser Ser
Phe Leu Gly Leu Arg Thr Ala Met Leu Asp Phe Arg Ser Leu Glu Met
Lys
SEQ ID NO: 20
Met Gly Arg Ser Pro Cys Cys Glu Lys Ala His Thr Asn Lys Gly Ala
Trp Thr Lys Glu Glu Asp Asp Arg Leu Val Ala Tyr Ile Arg Ala His
Gly Glu Gly Cys Trp Arg Ser Leu Pro Lys Ala Ala Gly Leu Leu Arg
Cys Gly Lys Ser Cys Arg Leu Arg Trp Ile Asn Tyr Leu Arg Pro Asp
Leu Lys Arg Gly Asn Phe Thr Ala Asp Glu Asp Asp Leu Ile Val Lys
Leu His Ser Leu Leu Gly Asn Lys Trp Ser Leu Ile Ala Ala Arg Leu
Pro Gly Arg Thr Asp Asn Glu Ile Lys Asn Tyr Trp Asn Thr His Ile
Lys Arg Lys Leu Leu Ser Arg Gly Ile Asp Pro Val Thr His Arg Pro
Ile Ala Asp Ala Ala Arg Asn Val Thr Ile Ser Phe Gln Pro Asp Ala
Pro Ser Gln Gln Gln Leu Ser Asp Asp Ala Glu Ala Pro Pro Pro Pro
Pro Pro Gln Gln Gln Gln Gln Leu Lys Pro Pro Pro Arg Cys Pro Asp
Leu Asn Leu Asp Leu Cys Ile Ser Pro Pro Cys His Lys Glu Glu Glu
Asp Gln Glu Leu Val Lys Pro Ala Ala Val Lys Arg Glu Met Leu Gln
Ala Gly His Gly Thr Leu Gly Leu Cys Phe Gly Cys Ser Leu Gly Leu
Gln Lys Gly Ala Ala Gly Cys Thr Cys Ser Ser Asn Ser His Phe Leu
Gly Leu Arg Val Gly Met Leu Leu Asp Phe Arg Gly Leu Glu Met Lys
SEQ ID NO: 37
atg gta cat tcg aag aag ttc cga ggt gtc cgc cag cgt cag tgg ggt
tct tgg gtt tct gag att cgt cat cct ctc ttg aag aga aga gtg tgg
cta gga aca ttc gac acg gcg gaa aca gcg gct aga gcc tac gac caa
gcc gcg gtt cta atg aac ggc cag agc gcg aag act aac ttc ccc gtc
atc aaa tcg aac ggt tca aat tcc ttg gag att aac tct gcg tta agg
tct ccc aaa tca tta tcg gaa cta ttg aac gct aag cta agg aag aac
tgt aaa gac cag aca ccg tat ctg acg tgt ctc cgc ctc gac aac gac
agc tca cac atc ggc gtc tgg cag aaa cgc gcc ggg tca aaa acg agt
cca aac tgg gtc aag ctt gtt gaa cta ggt gac aaa gtt aac gca cgt
ccc ggt ggt gat att gag act aat aag atg aag gta cga aac gaa gac
gtt cag gaa gat gat caa atg gcg atg cag atg atc gag gag ttg ctt
aac tgg acc tgt cct gga tct gga tcc att gca cag gtc taa
SEQ ID NO: 38
atg gta cag acg aag aag ttc aga ggt gtc agg caa cgc cat tgg ggt
tct tgg gtc gct gag att cgt cat cct ctc ttg aaa cgg agg att tgg
cta ggg acg ttc gag acc gca gag gag gca gca aga gca tac gac gag
gcc gcc gtt tta atg agc ggc cgc aac gcc aaa acc aac ttt ccc ctc
aac aac aac aac acc gga gaa act tcc gag ggc aaa acc gat att tca
gct tcg tcc aca atg tca tcc tca aca tca tct tca tcg ctc tct tcc
atc ctc agc gcc aaa ctg agg aaa tgc tgc aag tct cct tcc cca tcc
ctc acc tgc ctc cgt ctt gac aca gcc agc tcc cat atc ggc gtc tgg
cag aaa cgg gcc ggt tca aag tct gac tcc agc tgg gtc atg acg gtg
gag cta ggt ccc gca agc tcc tcc caa gag act act agt aaa gct tca
caa gac gct att ctt gct ccg acc act gaa gtt gaa att ggt ggc agc
aga gaa gaa gta ttg gat gag gaa gaa aag gtt gct ttg caa atg ata
gag gag ctt ctc aat aca aac taa
SEQ ID NO: 39
atg gta cat tcg agg aag ttc cga ggt gtc cgc cag cga caa tgg ggt
tct tgg gtc tct gag att cgc cat cct cta ttg aag aga aga gtg tgg
ctt gga act ttc gaa acg gca gaa gcg gct gca aga gca tac gac caa
gcg gct ctt cta atg aac ggc caa aac gct aag acc aat ttc cct gtc
gta aaa tca gag gaa ggc tcc gat cac gtt aaa gat gtt aac tct ccg
ttg atg tca cca aag tca tta tct gag ctt ttg aac gct aag cta agg
aag agc tgc aaa gac cta acg cct tct ttg acg tgt ctc cgt ctt gat
act gac agt tcc cac att gga gtt tgg cag aaa cgg gcc ggg tcg aaa
aca agt ccg act tgg gtc atg cgc ctc gaa ctt ggg aac gta gtc aac
gaa agt gcg gtt gac tta ggg ttg act acg atg aac aaa caa aac gtt
gag aaa gaa gaa gaa gaa gaa gaa gct att att agt gat gag gat cag
tta gct atg gag atg atc gag gag ttg ctg aat tgg agt tga
SEQ ID NO: 40
atg gga agg tct cct tgc tgt gag aaa gac cac aca aac aaa gga gct
tgg act aag gaa gaa gac gat aag ctc atc tct tac atc aaa gct cac
ggt gaa ggt tgt tgg cgt tct ctt cct aga tcc gcc ggt ctt caa cgt
tgc gga aaa agc tgt cgt ctc cga tgg att aac tat ctc cga cct gat
ctc aag agg ggt aac ttc acc ctc gaa gaa gat gat ctc atc atc aaa
cta cat agc ctt ctc ggt aac aag tgg tct ctt att gcg acg aga tta
cca gga aga aca gat aac gag att aag aat tac tgg aac aca cat gtt
aag agg aag cta tta aga aaa ggg att gat ccg gcg act cat cga cct
atc aac gag acc aaa act tct caa gat tcg tct gat tct agt aaa aca
gag gac cct ctt gtc aag att ctc tct ttt ggt cct cag ctg gag aaa
ata gca aat ttc ggg gac gag aga att caa aag aga gtt gag tac tca
gtt gtt gaa gaa aga tgt ctg gac ttg aat ctt gag ctt agg atc agt
cca cca tgg caa gac aag ctc cat gat gag agg aac cta agg ttt ggg
aga gtg aag tat agg tgc agt gcg tgc cgt ttt gga ttc ggg aac ggc
aag gag tgt agc tgt aat aat gtg aaa tgt caa aca gag gac agt agt
agc agc agt tat tct tca acc gac att agt agt agc att ggt tat gac
ttc ttg ggt cta aac aac act agg gtt ttg gat ttt agc act ttg gaa
atg aaa tga
SEQ ID NO: 41
atg gga agg tca ccg tgc tgt gag aaa gct cac aca aac aaa gga gca
tgg acg aaa gaa gag gac gag agg ctc gtc gcc tac att aaa gct cat
gga gaa ggc tgc tgg aga tct ctc ccc aaa gcc gcc gga ctt ctt cgc
tgt ggc aag agc tgc cgt ctc cgg tgg atc aac tat ctc cgg cct gac
ctt aag cgt gga aac ttc acc gag gaa gaa gac gaa ctc atc atc aag
ctc cat agc ctt ctt ggc aac aaa tgg tcg ctt att gcc ggg aga tta
ccg gga aga aca gat aac gag ata aag aac tat tgg aac acg cat ata
cga aga aag ctt ata aac aga ggg att gat cca acg agt cat aga cca
atc caa gaa tca tca gct tct caa gat tct aaa cct aca caa cta gaa
cca gtt acg agt aat acc att aat atc tca ttc act tct gct cca aag
gtc gaa acg ttc cat gaa agt ata agc ttt ccg gga aaa tca gag aaa
atc tca atg ctt acg ttc aaa gaa gaa aaa gat gag tgc cca gtt caa
gaa aag ttc cca gat ttg aat ctt gag ctc aga atc agt ctt cct gat
gat gtt gat cgt ctt caa ggg cat gga aag tca aca acg cca cgt tgt
ttc aag tgc agc tta ggg atg ata aac ggc atg gag tgc aga tgc gga
aga atg aga tgc gat gta gtc gga ggt agc agc aag ggg agt gac atg
agc aat gga ttt gat ttt tta ggg ttg gca aag aaa gag acc act tct
ctt ttg ggc ttt cga agc ttg gag atg aaa taa
SEQ ID NO: 42
atg gga aga tct cct tgc tgc gag aaa gaa cac atg aac aaa ggt gct
tgg act aaa gaa gaa gat gag aga cta gtc tct tac atc aag tct cac
ggt gaa ggt tgt tgg cga tct ctt cct aga gcc gct ggt ctc ctt cgc
tgc ggt aaa agc tgc cgt ctt cgg tgg att aac tat ctc cga cct gat
ctc aaa aga gga aac ttt aca cat gat gaa gat gaa ctt atc atc aag
ctt cat agc ctc cta ggc aac aag tgg tct ttg att gcg gcg aga tta
cct gga aga aca gat aac gag atc aag aac tac tgg aac aca cat ata
aag agg aag ctt ttg agc aaa ggg att gat cca gcc act cat aga ggg
atc aac gag gca aaa att tct gat ttg aag aaa aca aag gac caa att
gta aaa gat gtt tct ttt gtg aca aag ttt gag gaa aca gac aag tct
ggg gac cag aag caa aat aag tat att cga aat ggg tta gtt tgc aaa
gaa gag aga gtt gtt gtt gaa gaa aaa ata ggc cca gat ttg aat ctt
gag ctt agg atc agt cca cca tgg caa aac cag aga gaa ata tct act
tgc act gcg tcc cgt ttt tac atg gaa aac gac atg gag tgt agt agt
gaa act gtg aaa tgt caa aca gag aat agt agc agc att agc tat tct
tct att gat att agt agt agt aac gtt ggt tat gac ttc ttg ggt ttg
aag aca aga att ttg gat ttt cga agc ttg gaa atg aaa taa
SEQ ID NO: 43
atg gta cag cca aag aag aag ttt cgt gga gtc agg cag cgg cac tgg
ggc tcc tgg gtc tct gag atc aga cac ccc ctc ctt aaa agg agg gtg
tgg ctg ggc acc ttt gag acg gcc gag gag gct gcg cga gcc tac gat
gag gct gct gtg ctg atg agt ggc cgc aac gcc aag acc aac ttc ccc
gtg cag agg aac tcc acc ggt gat ctc gcc acg gcc gca gac cag gac
gcc cgt agc aat ggc ggt agc agg aac tcc tcc gcg ggc aac ctg tca
cag att ctc agt gct aag ctc cgc aag tgc tgc aag gcg cca tct ccg
tcc tta acc tgc ctc cgc ctc gac ccc gag aag tcc cac att ggc gtg
tgg caa aag cgc gca ggg gcc cgt gct gac tcc aac tgg gtg atg acg
gtg gag ctc aac aaa gag gta gaa cca act gaa cct gca gct cag ccc
aca tca aca gca aca gct tcg caa gtg aca atg gat gat gag gaa aag
att gcg ctg caa atg atc gag gag ttg ctg agc agg agc agt cca gct
tca ccc tca cat gga gag gga gag ggt agc ttt gtc atc tga
SEQ ID NO: 44
atg gga cag tcg aag aag aag ttc cgc gga gtc agg cag cgc cac tgg
ggc tcc tgg gtc tcc gag atc agg cac cct ctc ctt aag agg agg gtg
tgg ctg ggt acc ttt gag acg gcg gag gag gcg gcg cgg gcg tac gac
gag gcc gcc atc ctg atg agc ggc cgc aac gcc aag acc aac ttc cca
gtc gcg agg aac gcc acg ggg gag ctc aca ccg gcg gct gcg gtg gca
ggg cgg gat ggc cgt gtc ggc ggc ggc agc ggc agc tcg tcc tca atg
acg gcc aac ggc ggc ggg aac agc ctg tct cag atc ctc agc gcc aag
ctc cgc aag tgc tgc aag acg ccg tcg ccg tcg ctc acc tgc ctc cgc
ctt gac ccg gag aag tcc cac att ggc gtc tgg cag aag cgc gcc ggc
gca cgc gct gac tcc agc tgg gtc atg acc gtc gag ctc aac aag gac
acg gcc gtg tcg tcg gct gcg acg gtg gca gca gca aca gca gtg tcg
tcc agc gac cag ccg act ccg agt gac agc aca gtc aca acg acg tcc
acg tcc acc acg ggc tcg ccg tcg cca cca cct ccg gca atg gac gac
gag gag agg atc gcg ctg cag atg atc gag gag ctg ctg ggc agg agc
ggc ccg ggc tcg ccg tca cat ggg ctg ctg cac ggt ggt gaa ggt agc
ctc gtc atc tga
SEQ ID NO: 45
atg ggg agg tcg ccg tgc tgc gag aag gag cac act aac aag ggc gcg
tgg acc aag gag gag gac gag cgc ctc gtc gcc tac atc cgc gcc cac
ggc gag ggc tgc tgg cgc tcg ctc ccc aag gcc gcc ggc ctc ctc cgc
tgc ggc aag agc tgc cgc ctc cgc tgg atc aac tac ctc cgc ccc gac
ctc aag cgc ggc aac ttc acc gcc gac gag gac gac ctc atc atc aag
ctc cac agc ctc ctc ggc aac aag tgg tct ctg atc gcg gcg agg ctg
ccg ggg agg acg gac aac gag atc aag aac tac tgg aac acg cac atc
cgc cgg aag ctt ctc ggc agg ggg atc gac ccc gtc acg cac cgc ccc
gtc aac gcc gcc gcc gcc acc atc tcc ttc cat ccc cag ccg ccg cca
acg acg aag gag gag cag ctc ata ctc agc aag ccg ccc aag tgc ccc
gac ctc aac ctg gac ctc tgc atc agc ccg ccg tcg tgc cag gaa gaa
gac gat gac tat gag gcg aag ccg gcg atg atc gtg agg gcg ccg gag
ctg cag cgc cgc cgc ggc ggc ctc tgc ttc ggc tgc agc ctc ggc ctc
cag aag gag tgc aag tgc agc ggc ggc ggc gcc ggc gcc ggc gcc ggc
aac aac ttc ctc ggc ctc agg gct ggc atg ctc gac ttc aga agc ctc
ccc atg aaa tga
SEQ ID NO: 46
atg ggg agg tca ccg tgc tgc gag aag gca cac acc aac aag gga gca
tgg acc aag gag gaa gat gac cgg ctc att gcc tac atc aag gcg cac
ggc gaa ggt tgc tgg cga tcg ctg ccc aag gcc gcc ggc ctc ctc cgc
tgt ggc aag agc tgc cgc ctc cgg tgg atc aac tac ctc cgg cct gac
ctc aag cgc ggc aac ttc acc gag gag gag gat gag ctg atc atc aag
ctt cac agc ctt tta ggc aac aaa tgg tct ctg ata gcc ggg agg ttg
cca gga aga acg gac aac gag atc aag aac tac tgg aac acg cac atc
agg agg aag ctg ctg agc cgt ggc atc gac ccg gtg aca cac cgg ccg
atc aac gac agc gcg tcc aac atc acc ata tca ttc gag gcg gcc gcg
gcg gcg gcg agg gac gac aag gcc gcc gtg ttc cgg cga gag gac cat
cct cat cag ccg aag gcg gtg aca gtg gca cag gag cag cag gca gcc
gcc gat tgg ggc cat ggg aag cca ctc aag tgc cct gac ctc aat ctg
gac ctc tgc atc agc ctc cct tcc caa gaa gag ccc atg atg atg aag
ccg gtg aag agg gag acc ggc gtc tgc ttc agc tgc agc ctg ggg ctc
ccc aag agc aca gac tgc aag tgc agc agc ttc ctg gga ctc agg aca
gcc atg ctc gac ttc aga agc ttg gaa atg aaa tga
SEQ ID NO: 47
atg gga agg tct cct tgc tgt gaa aaa gct cat aca aac aaa ggc gca
tgg act aag gaa gaa gat gat cgc ctt att gct tac att aga acc cac
ggt gaa ggt tgc tgg cgt tca ctt cct aaa gct gct ggc ctt cta aga
tgc ggc aag agc tgc aga ctt cgt tgg atc aac tat tta aga cct gac
ctt aaa cgt ggc aat ttt act gaa gaa gaa gat gag ctc att atc aaa
ctc cat agt ctc ctc ggc aac aaa tgg tca ctt ata gcc gga agg tta
cca ggg aga aca gat aat gag ata aag aat tat tgg aac aca cat ata
aga agg aag ctc ttg aat aga ggc ata gat cct gcg act cat agg cca
ctc aat gaa cca gcc caa gaa gct tca aca aca ata tct ttc agc act
act acc tca gtt aaa gaa gag tcg ttg agt tct gtt aaa gag gaa agt
aat aag gag aag ata att agc gca gct gct ttt ata tgc aaa gaa gag
aaa acc cca gtt caa gaa agg tgt cca gac ttg aat ctt gaa ctt aga
att agc ctt cct tgc caa aac cag cct gat cgt cac cag gca ttc aaa
act gga gga agt aca agt ctt tgt ttt gct tgc agc ttg ggg cta caa
aac agc aag gac tgc agt tgc agt gtc att gtg ggt act att gga agc
agc agt agt gct ggc tcc aaa act ggc tat gac ttc tta ggg atg aaa
agt ggt gtg ttg gat tat aga ggt ttg gag atg aaa tga
SEQ ID NO: 48
atg gga agg tct cct tgc tgt gaa aaa gcc cat aca aac aag ggt gcg
tgg acc aag gag gaa gac gat cgc ctt gtt gct tac att aga gct cac
ggt gaa ggt tgc tgg cgc tca ctt cct aaa gcc gct ggc ctt ctt aga
tgt ggc aag agt tgc aga ctt cgt tgg atc aac tat tta aga cct gac
ctt aaa cgt ggc aat ttc acc gaa gca gaa gat gag ctc att atc aaa
ctc cat agc ctc ctt gga aac aaa tgg tca ctc ata gct gga aga tta
cca ggg aga aca gat aat gag ata aag aat tat tgg aac aca cat ata
aga agg aag ctt ttg aac aga ggc ata gat ccc gca act cat agg cca
ctc aac gaa cca gca gta caa gaa gcc aca aca aca ata tct ttc acc
acg act act act tca gta ctt gaa gaa gag tct ctg ggt tct ata att
aaa gag gaa aat aaa gag aag ata att agc gca act gct ttc gta tgc
aaa gaa gag aaa acc caa gtt caa gaa agg tgt cca gac ttg aat ctc
gag ctt gga att agc ctt cct tcc caa aac cag cct gat cat cac cag
cca ttc aaa act gga gga agt aga agt ctt tgt ttt gct tgc agt ttg
ggg cta caa aac agc aag gat tgc agc tgc aat gtt att gtg agc act
gtt ggg agc agt ggc agc act agc aca aag act ggt tat gac ttc ttg
ggc atg aaa agt ggt gtt ttg gat tat aga agt tta gag atg aaa taa
SEQ ID NO: 49
atg aca gag aat ctc cac tcc aag aaa atg gta cag cca aag aag ttt
cgt gga gtc cgg cag cgc cac tgg ggt tcc tgg gtc tcc gag atc agg
cat ccc ctc ctt aag agg agg gtc tgg ctg ggc acc ttc gag acc gct
gag gag gca gcg aga gca tat gac gag gct gcc gtg ctg atg agc ggc
cgc aac gcc aag acc aac ttc ccg gtc caa agg agc agc aca ggg gag
cca acc cca gct gcg gga agg gac gct cac agc aac gcc ggc agc ggc
tcc tct acc gcc aac ctg tcc cag att ctc agt gcg aag ctc cgc aaa
tgc tgc aag gcg cca tcg ccc tcc ctg acc tgt ctc cgc ctt gac cct
gag aag tcc cac att ggt gtt tgg cag aag cgt gca gga gcc cgt gct
gac tcc aac tgg gtc atg acc gtg gag ctc aac aaa ggt gca gca tcc
act gat gct gca tca cag tcc aca tca gca aca act gct cca cca gcc
acc ccg atg gat gac gag gag agg atc gcc ctg caa atg atc gaa gag
ttg ctg agc agc agc agc cca gct tca ccc tcg cac gga gat gac caa
ggt cgc ttc atc atc tga
SEQ ID NO: 50
atg gtg caa tca aag aag aag ttc cgc ggc gtc agg cag cgc cac tgg
ggc tcc tgg gtc tcc gag atc agg cac ccg ctg ctt aag agg agg gtg
tgg ctg ggc acc ttc gag acg gca gag gag gcg gcg cgg gcg tac gac
gag gcc gcc gtc ctc atg agc ggc cgc aac gcc aag acc aac ttc ccc
gtc cca agg acc gcc acc ggg gag ctg gcc ccc gtg ccg gcc gcg cgg
gac gca cgt ggc ggc ggc ggc tcg tcc tcc gcg gca gca gcg ccc ggc
ggc ggc acc agc aac ctg tcg cag atc ctc agc gcc aag ctc cgc aag
tgc tgc aag acg ccg tcg ccg tcg ctc acc tgc ctc cgc ctc gac ccg
gag aag tcc cac att ggc gtc tgg cag aag cgc gcg ggc gcg cgc gcc
gac tcc agc tgg gtc atg acc gtc cag ctc aac aag gac gtg ccg ccg
ccg gcg tcc tcc tcc ggc gag gag ccg gtg ccc agc gac gga ggc gca
gcg gcc acc acg ccc acg tcc act tcc acg tcg tcc acg gtc acg acg
acc ggc tcg cct cca cct gcg atg atg atg gac gac gag gag agg att
gcg ctg cag atg atc gag gag ctg ctg ggc agc tcg cac tca cat ggg
atg ttc cag ggt gca gcg ggc agc atc gtc atc tga
SEQ ID NO: 51
atg ggg cgg tcg ccg tgc tgc gag aag gcg cac acg aac aag ggc gcg
tgg acc aag gag gag gac gac cgc ctg gtg gcg tac atc cgc gcg cac
ggc gaa ggg tgc tgg cgg tcg ctg ccc aag gcg gcc gga ctg atg cgc
tgc ggc aag agc tgc cgc ctc cgc tgg atc aac tac ctc cgc ccc gac
ctc aag cgc ggc aac ttc acc gcc gac gag gac gac ctc atc atc aag
ctg cac agc ctc ctc ggc aac aag tgg tcg ctc atc gcc gcg cgg ctc
ccg ggg cgg acg gac aac gag atc aag aac tac tgg aac acg cac atc
cgg cgg aag ctg ctt ggc agg ggc atc gac ccc gtc acg cac cgc ccc
atc gcc gac gcc ggc gcc ggc acc gtc acc acc atc tcg ttc cag ccc
aac aaa ccc aac gcc gcc gtc gca gcg cag gcg cca caa cat cag ccg
atc aag gcg gtg gcg acg gcc gtc gtt aag gtg ccc agg tgc ccc gac
ctc aac ctc gat ctc tgc atc agc ccg ccg tgc caa cag aag gaa gac
gag gag ctg gac ctc aag ccc gcc gtc gtc gtc aag cgg gag gtg ctg
cag gcc ggc cat ggc ggc agc ctc tgc ttc ggc tgc agc ctg ggc atc
caa aaa gga gcc ccc ggg tgc agc tgc agc agc agc aac agc cac cac
cgc ttc ttg ggg ctc cgg tcc ggc atg ctc gac ttc aga ggc ctc gag
atg aag tga
SEQ ID NO: 52
atg ggg agg tcg ccg tgc tgc gag aag gcg cac acc aac aag ggc gcg
tgg acc aag gag gag gac gac cgc ctc gtg gcg tac atc aag gcg cac
ggc gag ggt tgc tgg cgc tcg ctg ccc aag gcc gcc ggc ctc ctg cgc
tgc ggc aag agc tgc cgc ctc cgg tgg atc aac tac ctc cgc ccc gac
ctc aag cgc ggc aac ttc acg gaa gag gag gac gag ctc atc atc aag
ctc cac agc ctc ctc ggc aac aaa tgg tcc ctg atc gct gga agg ctg
ccg gga agg acg gac aac gag atc aag aac tac tgg aac acg cac atc
cgg agg aag ctg ctg agc agg ggg atc gac ccg gtg aca cac cgc ccc
atc aac gag cac acg tcc aac ata acc atc tcg ttc gag gcg gcg gcg
gcc gcg cgt gac cgt gag gag aat aag ggc gcc gtg ttc cgg ctg gag
gag cac aac aag gcg acg gcg gcg gcg gcc gcc gcg atc ggc cgc gat
cat cat cag aac cac cac ccc gcc ggc gac tgg ggc cag ggg aag ccg
ctc aag tgc ccc gac ctc aac ctg gac ctc tgc atc agc ccg ccg gcg
gcg ccg tgc cag gag gag aag gcc atg gtg acg atg aag ccc gtg aag
cgg gag gcc ggg ctc tgc ttc agc tgc agc ctg ggc ctc ccc aag agc
gcc gac tgc aag tgc agc aac ttc ctc gga ctc agg acc gcc atg ctc
gac ttc aga agc ctc gag atg aaa tga
SEQ ID NO: 53
atg aca gag aat ctc cac tcc agg aaa atg gta cag cca aag aag ttt
cgt gga gtc cgg cag cgc cac tgg ggc tcc tgg gtc tct gag atc agg
cat ccc ctc ctt aag agg agg gtc tgg ctg ggt acc ttt gag acg gct
gag gag gca gcg aga gca tat gat gag gct gct gtg ctg atg agc gga
cgc aac gcc aag acc aac ttc cca atc caa aga agc agc aca ggg gag
cct acc cca gct gcg gga agg gac gcc cgc agc aac ttc agc agc ggc
tcc tct acc acc aac ctg tcc cag att ctc agt gcg aag ctc cgc aaa
tgc tgc aag gcg cca tca ccg tcc ctg acc tgt ctc cgc ctt gac cct
gag aag tcc cac att ggt gtt tgg cag aag cgt gca gga gcc cgt gct
gac tcc aac tgg gtc atg aca gtg gag ctc aac aaa gat gca gca tcc
act gat gct gca tca cag tcc aca tca gca aca act gct cca cca gcc
acg ccg atg gat gag gag gag agg atc gca ctg caa atg atc gaa gag
ttg ctg agc agc agc agc cca gct tca ccc tca aac gga gat gac caa
ggt cgc ttc atc atc tga
SEQ ID NO: 54
atg ggg cgg tcg ccg tgc tgc gag aag gcg cac acc aac agg ggc gcg
tgg acc aag gag gag gac gag cgg ctg gtg gcc tac gtc cgc gcg cac
ggc gaa ggg tgc tgg cgc tcg ctg ccc agg gcg gcg ggc ctg ctg cgc
tgc ggc aag agc tgc cgc ctg cgc tgg atc aac tac ctc cgc ccg gac
ctc aag cga ggc aac ttc acc gcc gac gag gac gac ctc atc gtc aag
ctg cac agc ctc ctc ggg aac aag tgg tcg ctc atc gcc gcg cgg ctc
ccg ggg cgg acg gac aac gag atc aag aac tac tgg aac acg cac atc
cgg cgc aag ctg ctg ggc agc ggc atc gac ccc gtc acg cac cgc cgc
gtc gcg ggg ggc gcc gcg acc acc atc tcg ttc cag ccc agc ccc aac
tcc gcc gcc gcc gcc gcc gcc gca gaa aca gca gcg cag gcg ccg atc
aag gcc gag gag acg gcg gcc gtc aag gcg ccc agg tgc ccc gac ctc
aac ctg gac ctc tgc atc agc ccg ccg tgc cag cat gag gac gac ggc
gag gag gag gac gag gag ctg gac ctc aag ccc gcc ttc gtc aag cgg
gag gcg ctg cag gcc ggc cac ggc cac ggc cac ggc ctc tgc ctc ggc
tgc ggc ctg ggc gga cag aag gga gcg gcc ggg tgc agc tgc agc aac
ggc cac cac ttc ctg ggg ctc agg acc agc gtg ctc gac ttc aga ggc
ctg gag atg aag tga
SEQ ID NO: 55
atg ggg agg tcg ccg tgc tgc gag aag gcg cac acc aac aag ggc gcg
tgg acc aag gag gag gac gag cgc ctg gtc gcg cac atc agg gcg cac
ggc gag ggg tgc tgg cgc tcg ctg ccc aag gcc gcc ggc ctc ctg cgc
tgc ggc aag agc tgc cgc ctc cgc tgg atc aac tac ctc cgc ccc gac
ctc aag cgc ggc aac ttc acg gag gaa gag gac gag ctc atc gtc aag
ctg cac agc gtc ctc ggc aac aag tgg tcc ctg atc gcc gga agg ctg
ccc ggc agg acg gac aac gag atc aag aac tac tgg aac acg cac atc
cgg agg aag ctg ctg agc agg ggg atc gac ccg gtg acg cac cgc ccg
gtc acg gag cac cac gcg tcc aac atc acc ata tcg ttc gag acg gaa
gtg gcc gcc gct gcc cgt gat gat aag aag ggc gcc gtc ttc cgg ttg
gag gac gag gag gag gag gag cgc aac aag gcg acg atg gtc gtc ggc
cgc gac cgg cag agc cag agc cac agc cac agc cac ccc gcc ggc gag
tgg ggc cag ggg aag agg ccg ctc aag tgc ccc gac ctc aac ctg gac
ctc tgc atc agc ccg ccg tgc cag gag gag gag gag atg gag gag gct
gcg atg aga gtg aga ccg gcg gtg aag cgg gag gcc ggg ctc tgc ttc
ggc tgc agc ctg ggg ctc ccc agg acc gcg gac tgc aag tgc agc agc
agc agc ttc ctc ggg ctc agg acc gcc atg ctc gac ttc aga agc ctc
gag atg aaa tga
SEQ ID NO: 56
atg ggg cga tcg ccg tgc tgc gag aag gcg cac acg aac aag ggc gcc
tgg acc aag gag gag gac gac cgc ctc gtt gcc tac atc cgg gcg cac
ggc gag ggg tgc tgg cgc tcc ctc ccc aag gcc gcg ggc ctg ctg cgc
tgc ggc aag agc tgc cgc ctg cgc tgg atc aac tac ctc cgc ccg gac
ctc aag cgc ggc aac ttc acc gcc gac gag gac gac ctc atc gtc aag
ctc cac agc ctc ctc ggc aac aag tgg tcg ctc atc gcc gcg cgc ctc
ccc ggc cgc acc gac aac gag atc aag aac tac tgg aac acg cac atc
aag cgc aag ctc ctc agc cgc ggc atc gac ccc gtc aca cac cgc ccc
atc gcc gac gca gcc aga aac gtc acc atc tcc ttc cag ccc gac gcg
ccg tcg cag cag cag ctc agc gac gac gcc gag gcg ccg ccg ccg ccg
ccg ccg cag cag cag cag cag ctc aag ccg ccg ccc agg tgc ccc gac
ctc aat ctc gac ctc tgc atc agc ccg ccc tgc cac aag gaa gaa gag
gac cag gag ctc gtc aag ccc gcc gcc gtc aag cgc gag atg ctg cag
gcc ggc cac ggc act cta gga ctc tgc ttc ggc tgc agc ctg ggc ctc
cag aag ggc gcc gcc ggg tgc acc tgc agc agc aac agc cac ttc ctg
ggg ctc agg gtc ggc atg ctc ctc gac ttc aga ggc ctc gag atg aag

TABLE 2
Examples of Vascular Xylem Tissue Targeting Promoters for Gene Combination

| Referenced | | | | Sequence | Protein | | |
|---|---|---|---|---|---|---|---|
| expression | | Gene | SEQ | Feature and | family | | |
| database¹ | Expression² | name | ID NO: | Location | (Pfam) | Species | Gene ID |
| Arabidopsis | 14279.9 | AtCTL2 | 21 | promoter | PF00182 | Arabidopsis | AT3G16920 |
| root | | | | (1) . . . (1000); | | | |
| transcripts | | | | 5′UTR | | | |
| | | | | (1001) . . . (1014) | | | |
| Arabidopsis | 11468.5 | AtLAC4 | 22 | promoter | PF00394 | Arabidopsis | AT2G38080 |
| root | | | | (1) . . . (1000); | | | |
| transcripts | | | | 5′UTR | | | |
| | | | | (1001) . . . (1296) | | | |
| Arabidopsis | 9693.64 | AtCesA4 | 23 | promoter | PF03552 | Arabidopsis | AT5G44030 |
| root | | | | (1) . . . (1000) | | | |
| transcripts | | | | | | | |
| Arabidopsis | 9591.84 | AtCesA8 | 24 | promoter | PF03552 | Arabidopsis | AT4G18780 |
| root | | | | (1) . . . (1000); | | | |
| transcripts | | | | 5′UTR | | | |
| | | | | (1001) . . . (1167) | | | |
| Arabidopsis | 8423.1 | AtFLA11 | 25 | promoter | PF02469 | Arabidopsis | AT5G03170 |
| root | | | | (1) . . . (1000); | | | |
| transcripts | | | | 5′UTR | | | |
| | | | | (1001) . . . (1019) | | | |
| Arabidopsis | 6779.81 | AtCesA7 | 26 | promoter | PF03552 | Arabidopsis | AT5G17420 |
| root | | | | (1) . . . (1000); | | | |
| transcripts | | | | 5′UTR | | | |
| | | | | (1001) . . . (1093) | | | |
| Arabidopsis | 6471.5 | AtIRX9 | 27 | promoter | PF03360 | Arabidopsis | AT2G37090 |
| root | | | | (1) . . . (1000); | | | |
| transcripts | | | | 5′UTR | | | |
| | | | | (1001) . . . (1146) | | | |
| Rice mas | 15061.86 | OsFLA9 | 28 | promoter | PF02469 | Rice | LOC_Os05g07060/ |
| transcripts | | | | (1) . . . (948); | | | Os05g0163300 |
| | | | | 5′UTR | | | |
| | | | | (949) . . . (1000) | | | |
| Rice mas | 10893.78 | OsCTL1 | 29 | promoter | PF00182 | Rice | LOC_Os09g32080/ |
| transcripts | | | | (1) . . . (677); | | | Os09g0494200 |
| | | | | 5′UTR | | | |
| | | | | (678) . . . (1000) | | | |
| Rice mas | 8754.03 | OsCesA4 | 30 | promoter | PF03552 | Rice | LOC_Os01g54620/ |
| transcripts | | | | (1) . . . (1000); | | | Os01g0750300 |
| | | | | 5′UTR | | | |
| | | | | (1001) . . . (1141) | | | |
| Rice mas | 7977.04 | OcCesA7 | 31 | promoter | PF03552 | Rice | LOC_Os10g32980/ |
| transcripts | | | | (1) . . . (1000) | | | Os10g0467800 |
| Rice mas | 7972.41 | OsLac10 | 32 | promoter | PF00394 | Rice | LOC_Os03g16610/ |
| transcripts | | | | (1) . . . (845); | | | Os03g0273200 |
| | | | | 5′UTR | | | |
| | | | | (895) . . . (1000) | | | |
| Rice mas | 7099.66 | OsGT43J | 33 | promoter | PF03360 | Rice | LOC_Os06g47340/ |
| transcripts | | | | (1) . . . (1000); | | | Os06g0687900 |
| | | | | 5′UTR | | | |
| | | | | (1001) . . . (1247) | | | |
| Maize leaf | 446.24 | ZmCesA10 | 34 | promoter | PF03552 | Maize | GRMZM2G445905 |
| gradient | | | | (1) . . . (977); | | | |
| transcripts | | | | 5′UTR | | | |
| | | | | (978) . . . (1000) | | | |
| Maize leaf | 123.92 | ZmCesA12 | 35 | promoter | PF03552 | Maize | GRMZM2G142898 |
| gradient | | | | (1) . . . (1000) | | | |
| transcripts | | | | | | | |
| Maize leaf | 44.89 | ZmCesA11 | 36 | promoter | PF03552 | Maize | GRMZM2G037413 |
| gradient | | | | (1) . . . (1000) | | | |
| transcripts | | | | | | | |

¹The Bio-Analytic Resource for Plant Biology, available online at http://bar.utoronto.ca/ and described in Toufighi et al, “The Botany Array Resource: e-Northerns, Expression Angling, and Promoter Analyses,” The Plant Journal 43: 153-63 (2005), each of which is hereby incorporated by reference in its entirety.
²Relative gene expression value in vascular tissues or xylem-related organ.

The sequences referenced in Table 2 are set forth below.

SEQ ID NO: 21
acgtacctcg tgtccaccgg tgactctatc cccggcgtta gaagtgatga tagtctcgtt
cccaagggaa atcagccttc gaattggaat tgatccctcc ggacattttg tgccgttcgt
gtgccagact tgccatccat ataatgcatc ttcttctttt tttcccgcag atggcatgtc
cgttggtctt tcctgtatca tttatttaca aaagaaaaat aaattaaaca tttattaagt
tccccccgta aaaaaaaaat atatatatat atatatatat aacacatgca tcataattgg
tatgtccgta ggtgtttcct tatcataact gaaccattgg taaactatcg gttccgttaa
agcataagac tagaaaaggc tcggtgcgac tcgctaccac gtttctaaag attttattta
gcaaattaac cccaatatat attttgctat gagggtctaa acaaactggt atatgagcca
tttacttacc acttattagt tccaagtatt tattttttgg gttaattaat gtttaaatta
ttggttgaca aaaaatataa aaataatggt taagttattg aaatgacttg agcaatctga
tgcaactgcg gataacatga actcattcga agtgacgtcc caaatatttg attctttgtt
tttattcctt tttgtcaagg tcaagattgg ccaaacattt tcaatatcta aatatattga
cattcatagc ctggaaaaga aaaaatatat ggttaaatta gttccaaagt attctagcag
caacaaaacc gctccaataa acgatttcca atttctatct caaacttgtt tcccaatcat
tagttataat ccgtccccta aaccaaaaaa aaatctaatt gtaaaggtgt tgcaatagat
taaccatttt tattttattt tggtaaaata gattaaccat tttgttagaa aaatgaagtt
taaaacattt acagttctac gtgtacatgc ttcgaccaat atgcttcgac caat
SEQ ID NO: 22
atacatgtca tgattttata attatgtata tataaatact aattgatgta tgaagtacgt
agataatgtt acgatctatt aatctattta cattaacttt taattagtgt tgagtaggga
aaattaacat ataaaccttt agcagttggt tgtattatta aaaataattt gaacttaaaa
tccaccttcg aaaagataaa tcaaacaagt ataaaaaatg ctataaatcc agaatattta
cctaaggttt ttattcttct acttaataat gtaagataaa accggcacaa tacttgttac
gtatgcatgg taggtaccgc aattgtgtaa gcaaatcggc acaatactaa ggttacatat
actaactaaa taaaacaatc tgatttcagt gacaccgtat atctaacctt tattcaaatc
caagggaaca tgacttgact tcttctgttg gaactaactc gatccctcaa ccatctccag
ggatagaaga gttagtaaaa tcaaacttga agtgaggaag taagcagttt aacgactcca
tatgactaca gttatataca aagttgggca caaagtacaa gtactaaata ctcaaagtca
gataataatt ttaataagta caaactatat atatgcagta caattattga gtatatataa
acgagactgg tgatttgggg cattgtccac cagggtgtta tatcccaatt gaaatttgaa
aatttaagtg tgtgagtgtt acgacaaaaa aaagtgtgtg aattgtaggc gcggtgaaaa
ggtaaattaa gattggaact agaaaaatag ttgaatatcc tttactaaaa gttgtcaatt
ccggttttag taaaaaaaaa ttttaaaata gaaattttat ccaaaagact tcaaacacac
atattcgcat atataacata agatatcatt ttttgtaaac agttaaaaag aaaaacacat
gttttttttt ttaatttaga aaaaaacatg ttattataca aaacagagtt ttgcccactt
ttaatatgtt atgaaaagaa aaatgatttt cttgggtttg gtcagagaga ttggttgtgg
taagaatggg aatcttaatt acaaagaatt ggattttggg tcgacctacc acctaaaacg
acgtcgcctc catctctggt ttccaaatct ctttctcctc tccctttata agcttgcgtt
ggccagtcgc tcatctcgaa aacagagaga aaaagactaa aaacacagtt taagaagaag
gagagataga gagagaagag aaagatagag agggag
SEQ ID NO: 23
aactagaaca cttcagataa attttgtcgt tctgttgact tcatttattc tctaaacaca
aagaactata gaccataatc gaaataaaaa ccctaaaaac caaatttatc tatttaaaac
aaacattagc tatttgagtt tcttttaggt aagttattta aggttttgga gactttaaga
tgttttcagc atttatggtt gtgtcattaa tttgtttagt ttagtaaaga aagaaaagat
agtaattaaa gagttggttg tgaaatcata tttaaaacat taataggtat ttatgtctaa
tttggggaca aaatagtgga attctttatc atatctagct agttcttatc gagtttgaac
tcgggttatg attatgttac atgcattggt ccatataaat ctatgagcaa tcaatataat
tcgagcattt tggtataaca taatgagcca agtataacaa aagtatcaaa cctatgcagg
ggagaagatg atgaaaagaa gagtgtgagc caatacaaag cagatttgag gacatggctt
acaagtcttg ggtacagagt ttggggagtg atgggtgcac aatggaacag cttctctggt
tgtccagttc ccaagagaac cttcaagctc cctaactcca tctactatgt cgcctgatta
aatcttattt actaacaaaa caataagatc agagtttcat tctgattctt gagtcttttt
tttctctctc cctcttttca tttctggttt atataaccaa ttcaaatgct tatgatccat
gcatgaacca tgatcatctt tgtgtttttt tttccttctg tattaccatt ttgggccttt
gtgaaattga ttttgggctt ttgttatata atctcctctt tctctttctc tacctgattg
gattcaagaa catagccaga tttggtaaag tttataagat acaaaatatt aagtaagact
aaagtagaaa tacataataa cttgaaagct actctaagtt
SEQ ID NO: 24
tacatcagtt tcatcatcta tcttgtttct tatagaagct cacaatcttc ttcctggtcg
agtttagaaa tgtcagagag agttgtttcc acagagacgt agaaacccat aactttagta
ttcttcaacc cttacaactt atctgagcaa aatcagaagg tcgaatttga tggatggttt
tgctgtattt ggtcaacggt tttatttgag acagtagacc agaggaaact cagatgtgat
gatgcaaaga ctgaattggt taagagtgta gattgatttg ttctaacatt gcaaatgtag
agtagaatta tgcaaaaaac gttaatgaac agagaagtga ttaagcagaa acaaaattag
agaagtgata ttatatctca aaatttattt ttggtacagc taaagctcaa attgttatag
agattagaga tattaaacca aatgacgagt gttttcttta gtagtaaacg gtgaaaattc
tcttctgaca aagacaatta aaattttagg tttaagactt taatatttgt cacaaattgt
catttaccta aataaaaaaa aaactaaata ttttttttag atacatatgt gtcttataat
tttaactata aattttaatt ttatgtctta aataattgtt tacactataa atttaaatat
tttaatgcta aaattaattt gattcaaaaa agtgatttta attcttattt ttcttataga
aagttggtga ttgaaaagat ttacttaaaa attataacaa cttcaatggt gaataacccg
acccgaataa accggatata acaacttcaa tgttagcttg atatagaaag tacggtgacg
cttaggaggc aagcaagcta gtatctgccg ctggttagag acaaagaaca tgtgtcactc
ctctcaacta aaactttcct tcactttccc gcaaaatcat ttcaaaaaag ctccaaattt
agcttaccca tcagctttct cagaaaacca gtgaaagaaa cttctcaact tccgattttt
cacaatccac caaacttttt ttaataactt tttttcctct tattacaaaa cctccactct
catggcttct caaacttgtt atccatccaa atctcaatcc ctaattaggg ttcatttctc
tgtttctcca aacaggggaa ttcgaag
SEQ ID NO: 25
tcttcttgca tcaatgatat caacaacaat gggtaataaa gaagctactt cgaaattata
tattttttcg tattctatat tgatcatcag tcttaagtgg tttggtttgt tgcagtgaag
aagaactatg tatggatcta cgccaccgtt cagttcggtt ttgtggtcct tttcgctcag
cttttctaca gagttgtaag atttgatgta atgtcacaga gaaaccttac tttgttgtca
cagagaaacc ttactttgtt gaagagtttt tgattcctca cactctctct cattaacttg
tgtgtaggtg aagcagccgg taatgtgcat tgtcttagcc actatgatcg gatttggact
caccatgacc ggcacaacag ctattaacga gtatttgaaa tggaggagaa gcaattccca
cctgccagaa gagccagcaa gtactcaggt ggtttgacag cagcgtagat cttttgagtg
aagctagagt ccctaaaggg ttggatcggt tttcaattaa ccggtcggga ttcggttttc
ggtttagctt taatcgactt gtctaggttg agatcagatt tggttttcaa tacttccaag
tctttttttt tttgccaact aaaatataag gaatgatgat aggcacacac atgacacata
aaatcataat gaacagtagt atgattagca atccatattt cttggataac acttcttcac
agcttttttg acaggtcact ataacacctt tttcagttca tttttcattt tcaatcctca
cccacccaaa ctctcccttc aaagcaatgt ctctcctctc tctttctcaa ttcaaacaaa
ctttattaaa cctaaaagaa acatttccaa tctctaatga cttagttgat agaatctcat
ttagttacct agtaataatc ttcacactag taagagaatc ctactcttca ccaaactaca
tctctctcta tataacaaac cccaaaacat ctcaacatac acacacaaca actacaaca
SEQ ID NO: 26
tgcgaacagt ttgattctgt ttttcttttt cctttttttg ggtaattttc ttataacttt
tttcatagtt tcgattattt ggataaaatt ttcagattga ggatcatttt atttatttat
tagtgtagtc taatttagtt gtataactat aaaattgttg tttgtttccg aatcataagt
tttttttttt tttggttttg tattgatagg tgcaagagac tcaaaattct ggtttcgatg
ttaacagaat tcaagtagct gcccacttga ttcgatttgt tttgtatttg gaaacaacca
tggctggtca aggcccagcc cgttgtgctt ctgaacctgc ctagtcccat ggactagatc
tttatccgca gactccaaaa gaaaaaggat tggcgcagag gaattgtcat ggaaacagaa
tgaacaagaa agggtgaaga agatcaaagg catatatgat ctttacattc tctttagctt
atgtatgcag aaaattcacc taattaagga cagggaacgt aacttggctt gcactcctct
caccaaacct taccccctaa ctaattttaa ttcaaaatta ctagtatttt ggccgatcac
tttatataat aagataccag atttattata tttacgaatt atcagcatgc atatactgta
tatagttttt tttttgttaa agggtaaaat aataggatcc ttttgaataa aatgaacata
tataattagt ataatgaaaa cagaaggaaa tgagattagg acagtaagta aaatgagaga
gacctgcaaa ggataaaaaa gagaagctta aggaaaccgc gacgatgaaa gaaagacatg
tcatcagctg atggatgtga gtgatgagtt tgttgcagtt gtgtagaaat ttttactaaa
acagttgttt ttacaaaaaa gaaataatat aaaacgaaag cttagcttga aggcaatgga
gactctacaa caaactatgt accatacaga gagagaaact aaaagctttt cacacataaa
aaccaaactt attcgtctct cattgatcac cgttttgttc tctcaagatc gctgctaatc
tccggccgtc cct
SEQ ID NO: 27
tctctaattg tcaagtatct tagtctagag ttaattactt aaatactaaa aggctgtcga
caaaatcaag cttgaatctc cttgtggtat cttcaactct tcgttgtctg cttacgagtg
gtttactcag taattatcta taatatgtta ttttttttcc ctcatctttt agttgttgtt
tcattacatt gaaaagcttg taatgtcttt atatggtata tatggatctt atgagtgagg
caagatccat gatgtttttg atcttagaat gtatatgatg atcttagaat gtatttgacc
gcccacaaat tattgttcat tgggattata tctctagtcc aactccaagc aatcgaaatg
ggtcctgctt ttaagaacaa cagtatatgt ttaagaataa taactttata tattctcgat
tttaagatct tttgacaaaa cctccttttc gttaggagcg tactaatttc caagtgtttg
attagtgggg tctccgtaaa tttatttaga gtttctatct atttattaat agctcaatta
attaatctat actgtatcta aacatcaatt tatatattta ctcttgagac caaaactgtc
aatttataac attggatagt ttcttaattc ttattatata tttttcaaac acttttcaag
actaatctcc acattaggta ctctctctag agataaaaat atttatcaaa aacattttta
tttatttatt aagtagtaga taaactactg tggcaaaatc gtaaatgtct aaatgctgat
gaattttttt tgctgctcca atctggttta gtgctccata tacatccacg gccaaaatga
atctatggcg gcattaagat tcattagtaa gcaacgatta tattaatata attgttttta
gcaatgattt tccgtaattt cccaaatatg tttcagttaa tgtgttccaa tcccaacaac
tggttgttgc aaaagaccac caacgcaagc aatcatcaaa catcaaaata atcttacctt
agcgaacaaa caataactac acaattctca taaagctctt atatatcact aacttcacac
agggtt ccacaaaaat aaaaacggaa ctcactcaag aaaccttctt ccttgaagag
attttgtttt
SEQ ID NO: 28
tacagggtct caagccagga tgacctcctt tgaaacgtac gagtggtaaa acagtacgaa
gaacatcaaa ttttcatgag aattttcata ggagacaggt taagagagaa cttcaagaga
ttggacctta tgttaacttc ttctagagat tggaccttat gttaactttc ttctataaaa
tattagtgaa gtgaggaaac ttctaaaaca attatatgga gtgatgaaaa aaattatttg
gtcagacggt aactatgagt actccataat ccgtataaga taataacatg gtaattctat
taggcgttcg ccaacgaagc cccaagcagc cacccaaggt agctaggcgg tgcctttgtc
cgtgtatcaa aaatctccat gcacgggagc cattccaaat aaaattttga agctccaagt
ttttgttccg aaggatcaaa tagaaaaatt atccgtagaa attgaatcct aacaaaaatt
ccccacaatt cctctaatta aaacgaggcc gaagcggctt cctgatcgga cggctggaag
gccatacatg tcctggcatt aattatcact caccttagat tattacagct cggagctaga
aagccctgca agttgcaatt aatggtgagt atgatctgat ctgcagcgaa atgatctatc
gatgtcccta gttaagcagt cattgtgtcc cttacccacc taaatccacg agtgtcagag
ctaagcgcga tcccgatcct taaaccccaa ccccactctc ttgctgctcc atcaagcaac
caaccccaat ccacacacca tacataatta catactacca gctaattaat tactaataat
gacttaatat tccatcatct cccagctcag ttatcacttc ttgatcacac cccctaccat
tgattaacct cttccatctc actctacacg cctatataat tagcctaatg atctcacttt
gcaaagcatc tcattgcact aaactgctca ctgcatttgc
SEQ ID NO: 29
actcgattcc attagattat tcacaaaccc atgtgaaccg tgactgtcag tcaggtgagg
aaacagatat tccaaaaatc atattctttc cttttgtatt aaccaaattc acacaaaggt
gatatttaaa actgacgaag atgaaaatta agttgacgca cgtaaaatga aaaagctatc
ggcatatgat taattaaatt taattattac aaacttgata aatgaatata tttgatattt
taaggcaact tctatataaa acttttttat atgaagagga ctgctacaaa acgggatccc
gttgcaacgg gataaggcat attaactatt ctcttgcatg agtatcacag atatcaggcc
ctaagtatca taggtaccag gtaacaggta tcacaggtat cgggtaccat acagctcgta
ccacaacagg tatcaggtac tatctcataa aaggtaacgt gataccgttg tctaggtttt
ctgttatatg aaacatattg tagtttaaaa aacgtgccaa cgaaaataga gataaaaatc
tgaatcttga tgagaaaatc atgcccaaat ttcaccctaa aacagtcaat ttcccgcgaa
aaaaagcaaa aaaaaaaaaa ctccagacag ttgttaaaag gggaaaaaaa aagacagaat
gctcagccgt cgagacacac acaacggcaa cgtcttacca gctcggagct ctctcgcttg
ctgcctcttc tcttcttcct cccgccaccg acaccacctc caccagcagc ttcgccttcg
ccgcgtcggc atctgcagtt gccacttcgc tttcttccac tccctcctcc tcctcgctta
cctcacactc ctccccccca atttcatccc ccacccacca ccagatcccc caccacctgc
cgcattctcc cccccaagat ccagatcgcc ggtcaccccc acgaactcgc tcgagatcca
gagagagaga gagaggcagt ttcttggttg attttcgagg
SEQ ID NO: 30
GAGTTAAATTGATATGGTTTTGTCTGTCAAGGTGCCGTTTCTATGGTTGGAGGAGGAAAATGAAATT
AGGGGAATAGAACAGTCGCAATCCAAAAGCTATTGTTCTCTCTTCTACGGTAGTTGATAGTTCGATA
CGTGTGTTTGATGTGAGAGACTGAGAGGAGTGCACGGTGCACCTGCCCCGTAAGGACGCGGAACTAC
ATTATCAGAGGCTAGGCCGGTACTGTTATACGCGACGTTCACGAATCACGATGCGTAAAAAAGTGAA
GCATGAAACAGTACTAGGCTCTCCACGGGCCACATCATGAAAAAACTGGTGCGACGCCACGCTTAAC
AATTTGTCGGTCGTAAACTCGTAAAGTTAAAAGCCCCACGACATTGTCAACCAATATCTCAGCACAT
GAACTTCTCTAACGAGTACAACGAAACCGCATCCGCAAAAGCGCCGTGAACCAAAGCTCTTGCCGTG
CTGTGCCGTGCCGGTGGCCGTGAACATGTGGAACACGAAGAACTCTTGCGCGAGATCGGAGCACCTG
ACCTCCCACCTCGCGTCCGGCCCGTCGCCGTCGCAGCAAGCGGGCTGTCAAAAACGACGCCACAGCG
CGAGCGCTCTCGCCGATCCGGCGGACCCACCCTCCTCACCTCGCGCACCAACCGCCCGTTCGCTAGT
CCGATCCCCCACCCCTCATCCCCCCTACGCCTTGCAGGTTACGCGCCTCGCCGCGGCCAACGCAAAC
CAAACCAAATCCCCCGTCACCTTCGCTTCGAAACCCCGCAAAACCCCATGGAAGAAAACACCGAACA
CCTGCCGCGCGCACGCCTCCTCCTCCCCCGCCTCTCCTCCTCTCTCCCGTTCCATGTCCGCTCAACC
TTGCTTCCATTCTTTCCATCCACCCGCCGATCGACGCGATGCCGACGCCCCAACCCCACCCACCGCC
TGCCAGCGCCACCCCACCTCGCGCCTCTGCGGCTATGGCTATATCACCATGCCTCCAACCTCCGGTA
CGCTTAGCCTCTCTCTCTCTCCCCCTCCCATTCTGCGCATTGCTCTCTGCGCGCGGTCGCGTGCTGC
TGCTCGCGGCGCCCCGGAGCGTCTCCTTTGGGGGAGAGGAGAGGAGAGGAGAGGAGAGGGGGGTGAG
CC
SEQ ID NO: 31
ttcaatgcag gatgacgcca agagggagaa accaagcaga ggtggacggt acaacgtgta
agtcaccgca aaacgttgca gcttggatag tggccatcga gtggtgtgcc gataccggcg
cctgttcttt acagcctcag ctagtgttgt tgtccgaggc aatttttccg acctattgtg
ttgctttcct ctctgatagc ttatggtaaa agatacaaag atgttgagga gtttgtacgc
cacttaattt tgctcgtaac atacattgac aatcaagagg agccatggca ttgcgatctg
cttacacggc atattcttac tggatggtgt acactactta ccctttttaa tgcaagcatc
aatccattgc ttttctcact gcacacctga ttcgtactga aaacgtgaaa cataaaaaaa
aacaaaaatc tagctgatgt tggctctcgg ggcctcgagt ctagtttgtc ctagatggct
aacctgatat gtgttggtca cgctcacgtt tgaaccgaga aagagtgtgt gtgtgtgtgt
gtcggcgtgc tgctacacca gagcctccct gaatcgcaat gcgtgttaac gccagcatcg
caggatttca tctcacttga caggttcaga tggccttcct cctaccgtct gccatttata
cacgcagtga cttaacgctt acacgagccg gatggcccgg atctcccccc tgcaccatct
caccagaaaa acggtgaggc gtcaccgcaa cccacccacc aaacacatcc acgtcccttc
accgttggcc ttcgattttg cttcagctgc actacgaccc ctccaacaca tttccctcgc
gtctcgttgc gatctcacct tacgacgatc tcgttccagc agcagcagca tcggcagcgg
cggcttgctt ccgaagcgag caatgcatgg cgcgcgcggc cgcgtgcgtg cgtgccttgg
cttgcgctct aatcaaaccg ggacgcccca actcacggtt
SEQ ID NO: 32
acaacccctg ttgcaccaaa cttgcttttt taagttttaa ctgaaattag gatagcaaag
agagtacttt aggcttcatg ctacgagctg cctacgacca tagacaggct taatcttgtc
ttatagttgt atcttgttga tcattaggtg cctaactgcc tacataggca taatgcatcc
tttcatctga tctttgtcac tgaccccatg tacagagtcc tataagttgc aatgttctaa
tccttgtttc gtcagtcatt tcgtacacat ggatgaaagt ctggggttta accaccgatg
cgatccaatc ttctcctaca gtcgatgata aggaaatcgt aacaaagaat gtgaattttt
ttgacgctac aaagaatgtg aatgatctga ccagtctatc tttcaccgaa ctgaagcata
tactctgaat gtctaagatc atacttagac tgaactatat actctgaata tctaaagatc
tcatacattc atacttacgc tgcaggttgc aaattctagt cattattaca ctcgagacct
aaattatgat tagtggggtg tactccgata gaacagttta cagttcagaa ctcaaaagct
acgaatgaat tcatgacaaa aggcgacaag tgatacgtat tcgagaataa atgtgtgaac
aaaggccgtc tcaaaaaaaa aaaagaaaaa aaaaaagaga atccctttgc ctgcactcta
aaacccagcc cgacccaact ctttgtacat gaccagcaaa agcaccgtct gctgcgactt
tttttctctt gtgcaatcta ttgtcgggaa aaaagagagg agaattatca tatcatcacc
taataaattg caaccaccag aggtactgtc ctctctatat aactctttct cgggcatttt
gctggcactt gcctgttcta gtatctatag ctagctaact gttactgtac ctcctcccat
atatcatctt catatttttg cagatcgata agcgagaaaa
SEQ ID NO: 33
CATACTTTACCTTGTTGTATAACTGCATGCATAAGAATCTGAGAGCCATTGCTCAATTCTTTTCAACGAA
GATGTGAACTGTTGGAAGGCAATGTAAAACGGGAAGCGCTGTATGAAAGAATATGACGCACATCGTCTTT
TGTTTTTTAAGAATTGAGTATATATTCGTTGTGGGGAACAGCCTGATGATGGGCCCCGGGAATTAACGCT
CGAGCAACGTTGGACCATTCTGACATCGCGTTTCCTGATTAGCACAATGTTTCGTTTTATTTGGAAATTG
AATTGAATGTTTCTACTGTTATTAATTGCAGACAGTACACCAAACGACCAAATCTATCTGCAAACAATTA
ACCAAGACCAACTGGAGAATTTACAGATGAATCACTGTGTTACACCTGTAAACTGTGGCTCCTTTGAGAA
TTGAGTTACAACAAGAGTTTGGAGATGAACTTGTAGTTCATCTATATCATCTTAATTAAACAATAATATT
TATTCAGGAATGCAGTTCAGAGACTGCTTAACACACACACACACAAAAAAAAAACCTAAACCTGAGGCTT
GTACTGGAACAAGGTCATTAGCAAGGTGTCCTCTAGACTCCCGGACCGACACTACCCTTGGAAGTCAAAC
GCAGCTGGCACAAACAAACGGAGCCTCGGTGACGCCGGTAAACCGCACCAATCATTGTTAAACCAAAAAA
CGTGAACAACAAACCAAAAAGAAACTAAAAAACCGCTAAAAAGACGCAAAAGAGAGAGAAAAAAAATGAA
AGAAGAAAAGAAACGACGCGGACTCGCTGACGACCCGCGGCCCGGTCCAACCCAACCCCCACCGCCTCTC
TCGCCGACCCCGTCCACTCCGCCGAATTCCCCCCCAAACCCAAACCCAACGCGACCTCACCTCACCCGCA
CGACGACGGCACGACGCGACGCGTTGCCCGAGCTGACGGCTTGACGACGCCTCCGTCCCCGTCCGGCACC
AACCCATCCCAACCCAACGCTCCCCTTTTCCACTGACCAATTGATAGCCCAACCTCACCTCTCCTCTCCT
CCTCCCCCCTCCTCTCCTCCTCGCCTCCGCACCAGCAGTTTCGTGCACCGCACTTCACCCACCTACCTCC
CCCAACCTCCCCATCAAAAACTAGTAGTAGCAGTATCCACCCATCCACGCACGCGACCGAGCGCGATCGA
TTCGAGGCGGCGGCGGCGGAGGAGGAGGAGGGGGAGTAGATCCGGCGGGCGGCAGCG
SEQ ID NO: 34
tttgtgctga gatcggcacc agctttcatt taatacagcc tcagcttacc tgaggcaatt
ttcgcacctg ttatgatgtt gttttgctct cagataggtt tatgtagcac aagaaagata
tgttggagac gttgacgatt ttgtatgcaa ctaaatttct atcttaatat gccccgattc
aacagcaccc agtcgagtca ttgcgttctg gagattcttg cagcgcattt ccatgtttaa
gaccttatta tgaaatgtct ggcattcgtg gatccactga gcttctttct gcgaatgtgc
catatcgtgg cattggccga agcaaccaaa catttgttgc ccttttgtgt cggtgtttta
taaagtacct caatgacgat acagcctcag ggcgcttcct gcttttgcac ttattcggag
ttcaggcgag ttaacgaagt tcagacggtt ctgaagagag gccgtgttgt gttttgtcgg
cgtggtatcg cgcaagcaca tgtgtctttg gtaagatggt ctggatggct gtcctaccac
ctgccattta tacacacact gacttcaccg tcacactggc acgacatgag ctcgccatcc
taccagaaac gctgagacgt caccggcaac cacccctctc gctcgctctg gcctctgctc
ctgatttgat ttggacagaa aactgggcag ggcagggcgc gctcagcacg tttgcttcgg
aaacactgcg agtgtgcgac acatttcccg gcttgatctc gaagcgagcc ctgatgtgtt
tgtcatgcac ctgcctgcct tggcttgtgc tctaatcaac gccggactcc ccaactcacg
gttggtgcgg gacgccaccc cgccacctta ccgcccgcct cggcgcctca ccagtcacca
cacctcgcgc ctgccatcag ctatatcacc gtggccactt ccgtgtccct tcacggatac
ctcaccccca cagcccccgg tcgatcgctc ggcaatcggc
SEQ ID NO: 35
acatgtatat aagtattcaa tacataaatc agttctggta aagagtgaat taagttaatg
gacgtgctga aaatggtttg gcttagtctt gttgtgtttt atggaaaaat tgtgtaggtc
acgattactt ctcaatgcaa ttgagaaaag ttttaattgc aagccattta tggttattta
ttaaaaaaac aaggagtaac acgtcattgt tcaaggcgcc aagctaccac atcactatga
ttcaaagaac cacttcagat tttactcaag attggaaatt gaatggtttt gatatttaag
gttacttttt accgaaaaat attggaaacg atcagactga aatcgacttg atctagaaat
tatgatagaa tctcttgttt catagcgggt ccggtgggaa gacaaaatgt gtaatcccgt
cgatatgtgc tttctaaatg ctaaaaacga tccaatatac gacgtgtgct ttctagcctg
tttgtttgtc tttaatctgt actagtttct atgttttttt tctcattgaa ttacagctac
agtagtctaa acaacagcgg gctttaattc gaagcgaaca acacctgctg agtaagcaaa
cccacgctga atagtttcag aaatgttttc tggatgaata gcaaaattgt agtagcaaca
ggatagacgg cgggaaagcc aagtctcggt tggtccggcc gtccggacgc agcctgacaa
gggcagcagc atagcaatag catcaggcgc aagccagcgc aggcggcttt cgcttcactt
agcggcaacg gggacgcagc gcccgcaccc aaccaacacg agctcctctc ctcacccgcc
gcgacacgcg cgcggctcca acgcctccat ctccatcgcg cgcaccaaat cgcactccgt
ccgccccgtc gatcgaacag ccaccgctca cctctctcac ccgccaaaac ctcctcccct
ggcctcctct catactcata tagctgagca gcccctgcca
SEQ ID NO: 36
catgtatata agtattcaat acataaatca gttctggtaa agagtgaatt aagttaatgg
acgtgctgaa aatggtttgg cttagtcttg ttgtgtttta tggaaaaatt gtgtaggtca
cgattacttc tcaatgcaat tgagaaaagt tttaattgca agccatttat ggttatttat
taaaaaaaca aggagtaaca cgtcattgtt caaggcgcca agctaccaca tcactatgat
tcaaagaacc acttcagatt ttactcaaga ttggaaattg aatggttttg atatttaagg
ttacttttta ccgaaaaata ttggaaacga tcagactgaa atcgacttga tctagaaatt
atgatagaat ctcttgtttc atagcgggtc cggtgggaag acaaaatgtg taatcccgtc
gatatgtgct ttctaaatgc taaaaacgat ccaatatacg acgtgtgctt tctagcctgt
ttgtttgtct ttaatctgta ctagtttcta tgtttttttt ctcattgaat tacagctaca
gtagtctaaa caacagcggg ctttaattcg aagcgaacaa cacctgctga gtaagcaaac
ccacgctgaa tagtttcaga aatgttttct ggatgaatag caaaattgta gtagcaacag
gatagacggc gggaaagcca agtctcggtt ggtccggccg tccggacgca gcctgacaag
ggcagcagca tagcaatagc atcaggcgca agccagcgca ggcggctttc gcttcactta
gcggcaacgg ggacgcagcg cccgcaccca accaacacga gctcctctcc tcacccgccg
cgacacgcgc gcggctccaa cgcctccatc tccatcgcgc gcaccaaatc gcactccgtc
cgccccgtcg atcgaacagc caccgctcac ctctctcacc cgccaaaacc tcctcccctg
gcctcctctc atactcatat agctgagcag cccctgccac

Twenty-five independent plasmids suitable for Agrobacterium-mediated plant transformation were generated (see Table 3). Nine (9) combinations (construct 001 to construct 009 shown in Table 3) and one vector control were integrated into a dicot binary vector for alfalfa, canola, and other dicot transformation, and sixteen (16) combinations (construct 010 to construct 025 shown in Table 3) and one vector were generated into a monocot binary vector for sorghum, switchgrass, and other monocot transformation.

TABLE 3
List of Constructs

| | | Promoter and 5′UTR | | | Coding sequence | | | |
|---|---|---|---|---|---|---|---|---|
| Construct | SEQ | Gene | SEQ | | Gene | SEQ ID NO: | | |
| number | ID NO: | name | ID NO: | Gene ID | name | AA | NT | Gene ID |
| 001 | 57 | AtCTL2 | 21 | AT3G16920 | AtMYB32 | 4 | 40 | At4g34990 |
| 002 | 58 | AtLAC4 | 22 | AT2G38080 | AtMYB32 | 4 | 40 | At4g34990 |
| 003 | 59 | AtCesA4 | 23 | AT5G44030 | AtMYB32 | 4 | 40 | At4g34990 |
| 004 | 60 | AtCesA8 | 24 | AT4G18780 | AtMYB32 | 4 | 40 | At4g34990 |
| 005 | 61 | AtFLA11 | 25 | AT5G03170 | AtMYB32 | 4 | 40 | At4g34990 |
| 006 | 62 | AtCesA7 | 26 | AT5G17420 | AtMYB32 | 4 | 40 | At4g34990 |
| 007 | 63 | AtIRX9 | 29 | AT2G37090 | AtMYB32 | 4 | 40 | At4g34990 |
| 008 | 64 | AtCesA4 | 23 | AT5G44030 | AtMYB4 | 5 | 41 | At4g38620 |
| 009 | 65 | AtCesA8 | 24 | AT4G18780 | AtMYB4 | 5 | 41 | At4g38620 |
| 010 | 66 | ZmCesA12 | 35 | GRMZM2G142898 | ZmMYB31 | 19 | 55 | GRMZM2G050305 |
| 011 | 67 | ZmCesA11 | 36 | GRMZM2G037413 | ZmMYB31 | 19 | 55 | GRMZM2G050305 |
| 012 | 68 | OsCesA4 | 30 | LOC_Os01g54620/ | ZmMYB31 | 19 | 55 | GRMZM2G050305 |
| | | | | Os01g0750300 | | | | |
| 013 | 69 | OcCesA7 | 31 | LOC_Os10g32980/ | ZmMYB31 | 19 | 55 | GRMZM2G050305 |
| | | | | Os10g0467800 | | | | |
| 014 | 70 | ZmCesA12 | 35 | GRMZM2G142898 | ZmMYB42 | 19 | 55 | GRMZM2G419239 |
| 015 | 71 | ZmCesA11 | 36 | GRMZM2G037413 | ZmMYB42 | 19 | 55 | GRMZM2G419239 |
| 016 | 72 | OsCesA4 | 30 | LOC_Os01g54620/ | ZmMYB42 | 19 | 55 | GRMZM2G419239 |
| | | | | Os01g0750300 | | | | |
| 017 | 73 | OcCesA7 | 31 | LOC_Os10g32980/ | ZmMYB42 | 19 | 55 | GRMZM2G419239 |
| | | | | Os10g0467800 | | | | |
| 018 | 74 | ZmCesA12 | 35 | GRMZM2G142898 | PvMYB4 | 20 | 56 | Pavir. J16675 |
| 019 | 75 | ZmCesA11 | 36 | GRMZM2G037413 | PvMYB4 | 20 | 56 | Pavir. J16675 |
| 020 | 76 | OsCesA4 | 30 | LOC_Os01g54620/ | PvMYB4 | 20 | 56 | Pavir. J16675 |
| | | | | Os01g0750300 | | | | |
| 021 | 77 | OcCesA7 | 31 | LOC_Os10g32980/ | PvMYB4 | 20 | 56 | Pavir. J16675 |
| | | | | Os10g0467800 | | | | |
| 022 | 79 | ZmCesA12 | 35 | GRMZM2G142898 | OsSHN1 | 7 | 43 | LOC_Os06g40150/ |
| | | | | | | | | Os06g0604000 |
| 023 | 79 | ZmCesA11 | 36 | GRMZM2G037413 | OsSHN1 | 7 | 43 | LOC_Os06g40150/ |
| | | | | | | | | Os06g0604000 |
| 024 | 80 | OsCesA4 | 30 | LOC_Os01g54620/ | OsSHN1 | 7 | 43 | LOC_Os06g40150/ |
| | | | | Os01g0750300 | | | | Os06g0604000 |
| 025 | 81 | OcCesA7 | 31 | LOC_Os10g32980/ | OsSHN1 | 7 | 43 | LOC_Os06g40150/ |
| | | | | Os10g0467800 | | | | Os06g0604000 |

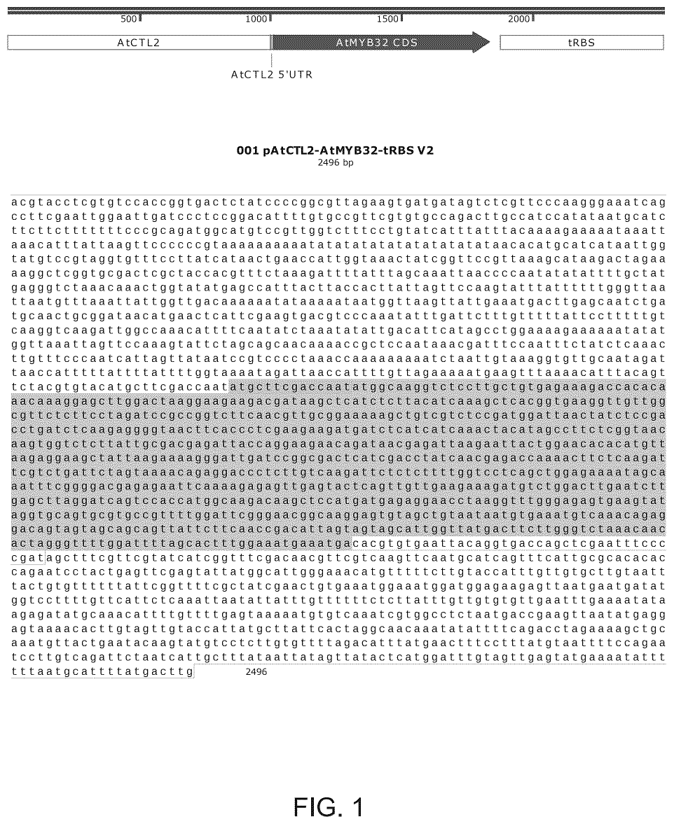

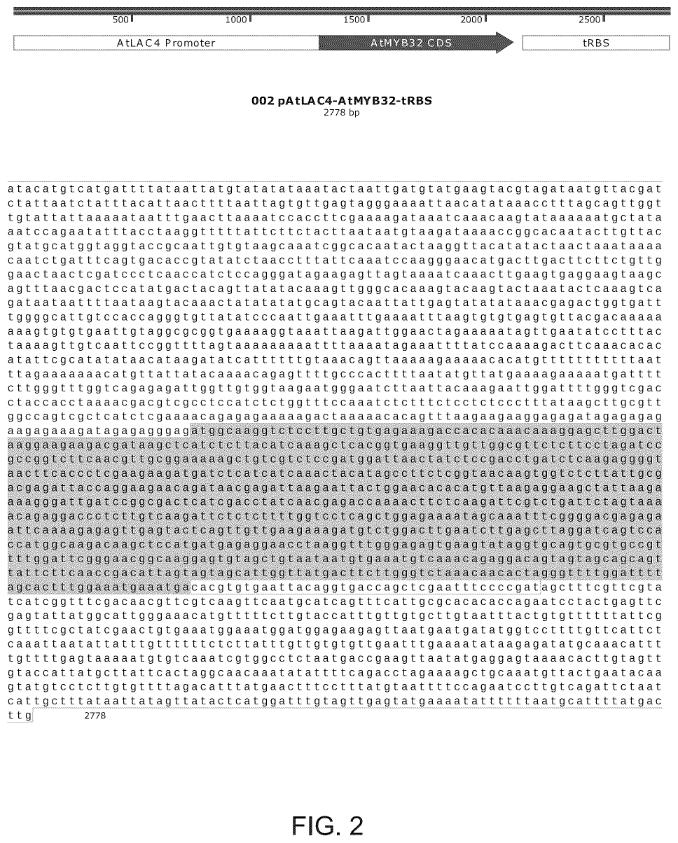

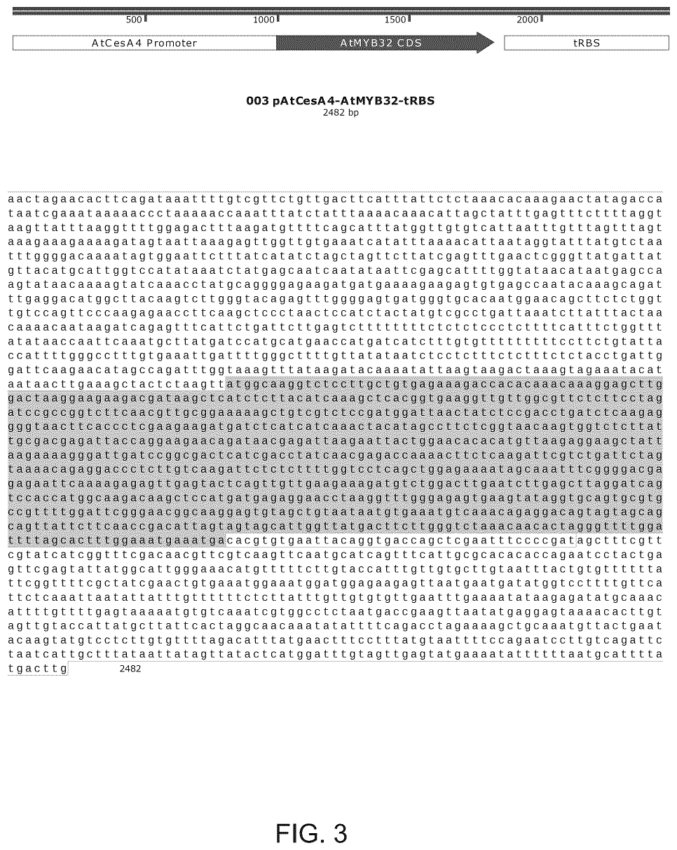

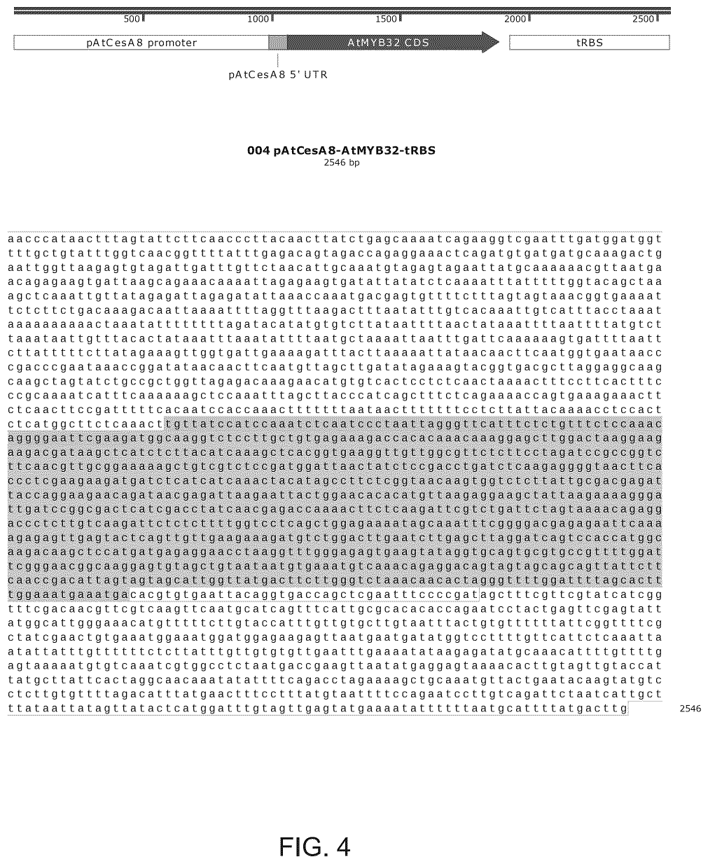

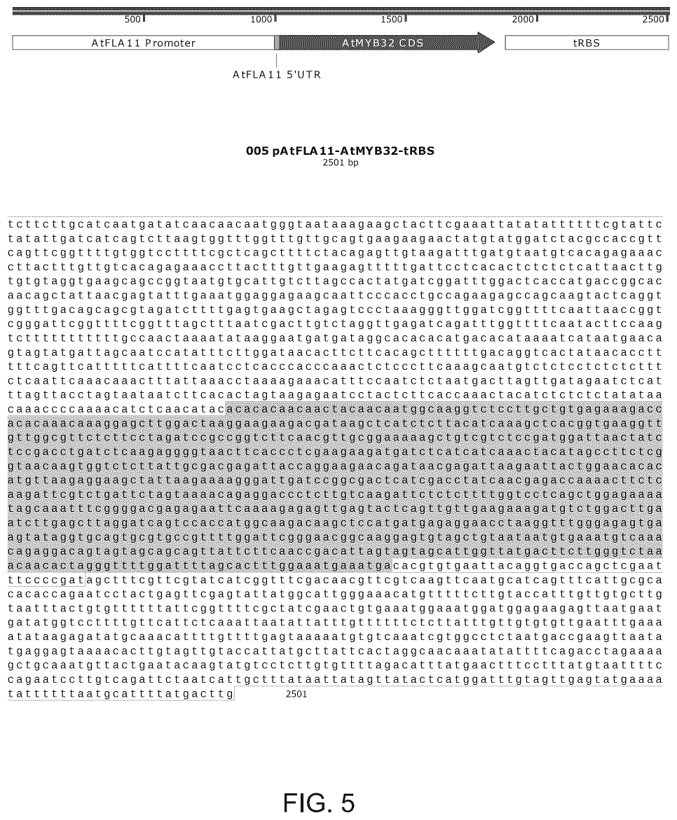

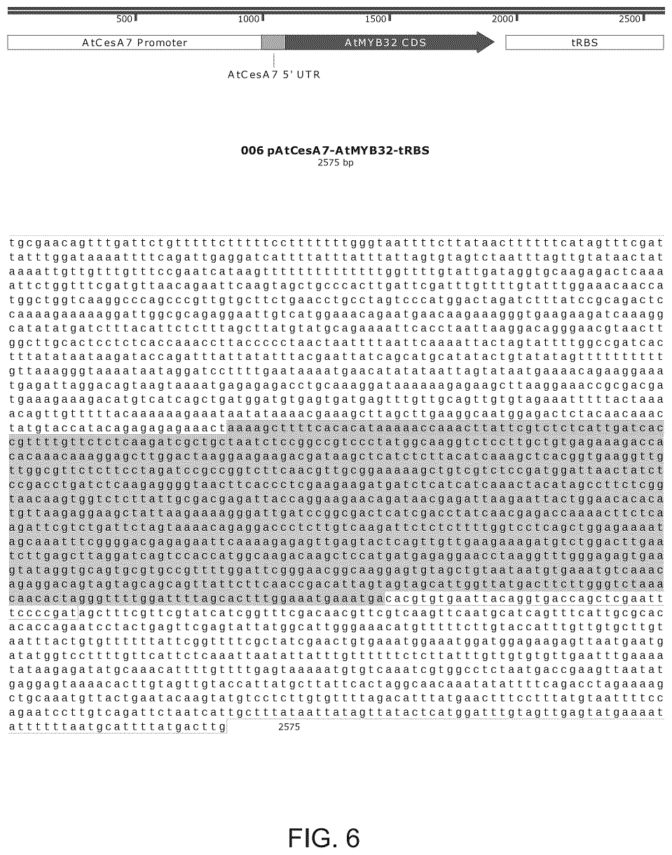

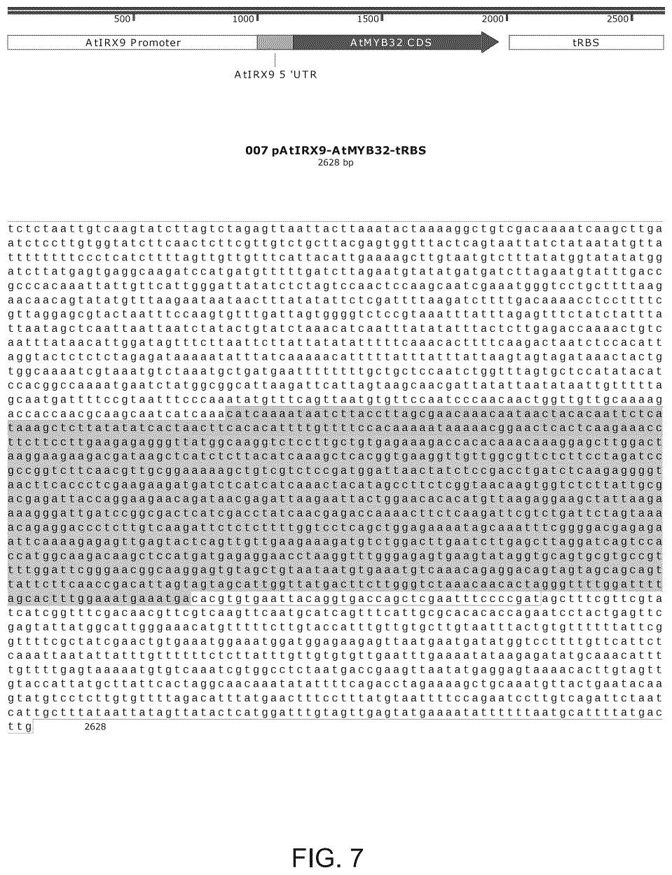

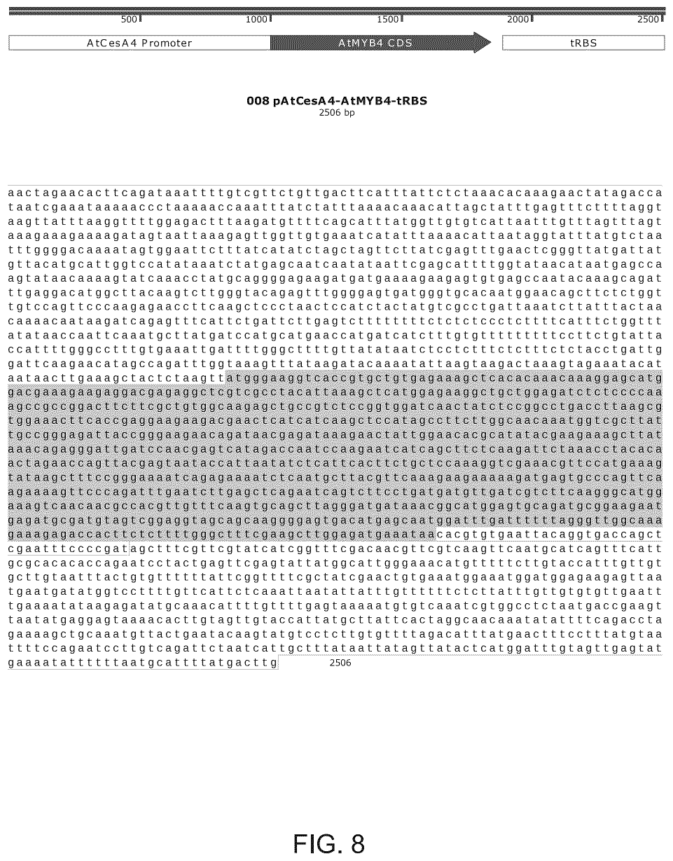

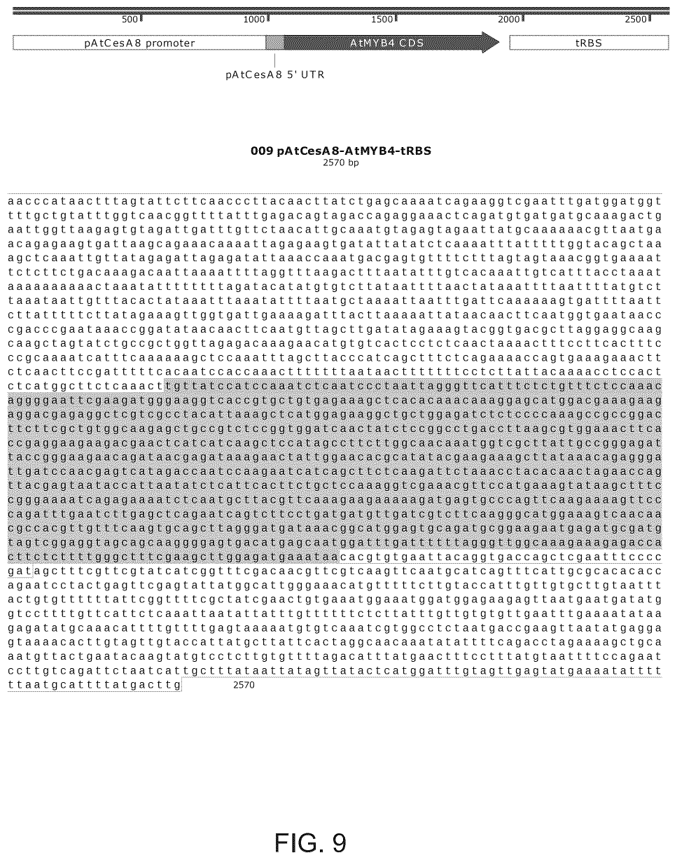

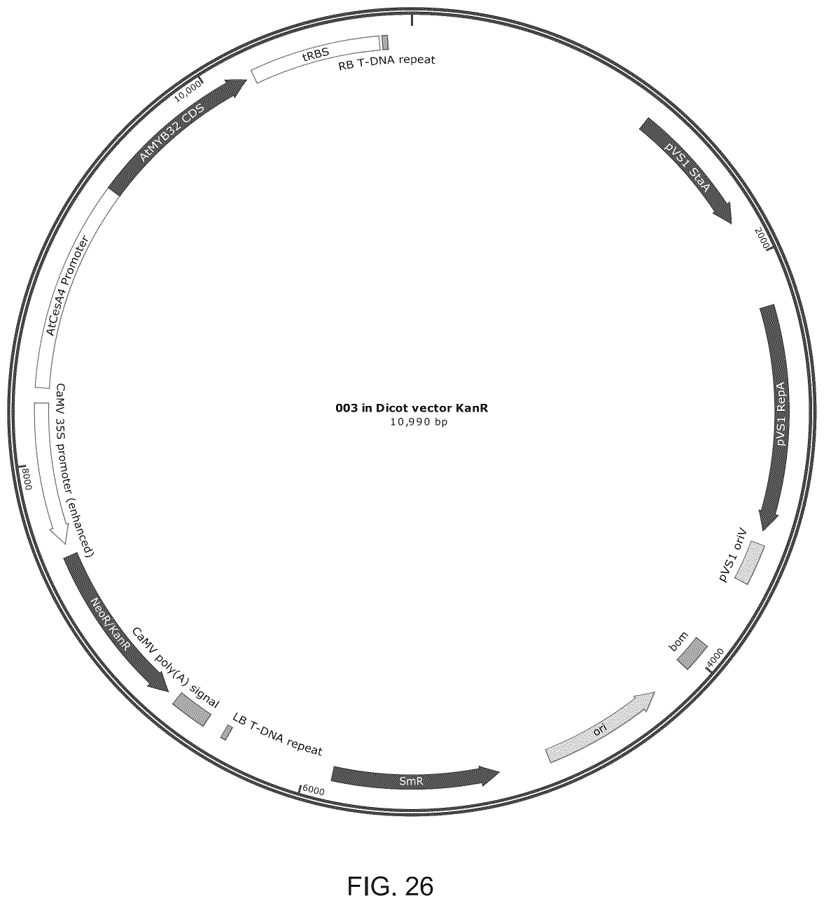

A similar approach was used for preparation of each of the constructs and plasmids. Briefly, approximately 1.0 kb of genome sequences upstream of the respective AtCTL2 (AT3G16920), AtLAC4 (AT2G38080), AtCesA4 (AT5G44030), AtCesA8 (AT4G18780), AtFLA11 (AT5G03170), AtCesA7 (AT5G17420), and AtIRX9 (AT2G37090) start codons were amplified by PCR from Arabidopsis thaliana genomic DNA; and the nucleotide sequences from start codon to stop codon of transcription factor AtMYB32 (At4g34990) and AtMYB4 (At4g38620) coding region were amplified by PCR from Arabidopsis thaliana cDNA derived from reverse transcription reaction using stem tissue RNA. The two nucleotide sequences (promoter and coding regions) were then combined with RBS terminator region into a commonly used binary vector plasmid (including Kanamycin selection marker) through Gibson cloning (Gibson et al., “Enzymatic Assembly of DNA Molecules up to Several Hundred Kilobases,” Nature Methods 6(5):343-345 (2009), which is hereby incorporated by reference in its entirety). The result was assembly of constructs 001-009: pAtCTL2-AtMYB32-tRBS (FIG. 1), pAtLAC4-AtMYB32-tRBS (FIG. 2), pAtCesA4-AtMYB32-tRBS (FIG. 3), pAtCesA8-AtMYB32-tRBS (FIG. 4), pAtFLA11-AtMYB32-tRBS (FIG. 5), pAtCesA7-AtMYB32-tRBS (FIG. 6), pAtIRX9-AtMYB32-tRBS (FIG. 7), pAtCesA4-AtMYB4-tRBS (FIG. 8), and pAtCesA8-AtMYB4-tRBS (FIG. 9), respectively. A representative plasmid map for construct 003 is shown in FIG. 26. Similar dicot-functional plasmids containing constructs 001, 002 and 004-009 were also prepared.

Using this same approach, approximately 1.0 kb of genome sequences upstream of the respective ZmCesA12 (GRMZM2G142898) ZmCesA11 (GRMZM2G037413), OsCesA4 (LOC_Os01g54620), and OcCesA7 (LOC_Os10g32980) start codons were amplified by PCR from corn and rice genomic DNA; and the nucleotide sequences from start codon to stop codon of transcription factors ZmMYB31 (GRMZM2G050305), ZmMYB42 (GRMZM2G419239), PvMYB4 (Pavir. J16675), and OsSHN1 (LOC_Os06g40150) were amplified by PCR from corn, rice, and switchgrass cDNA derived from reverse transcription reaction using stem tissue RNA.

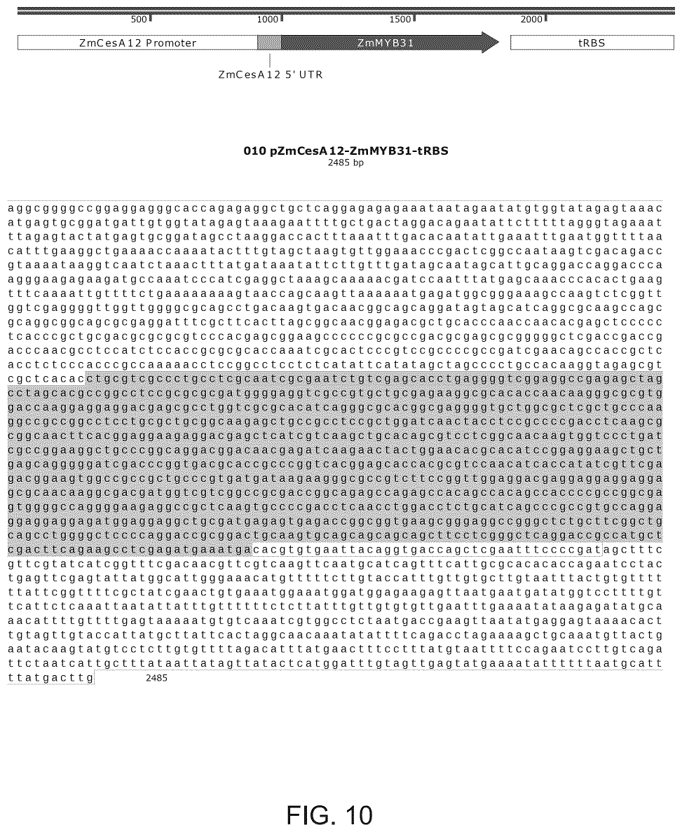

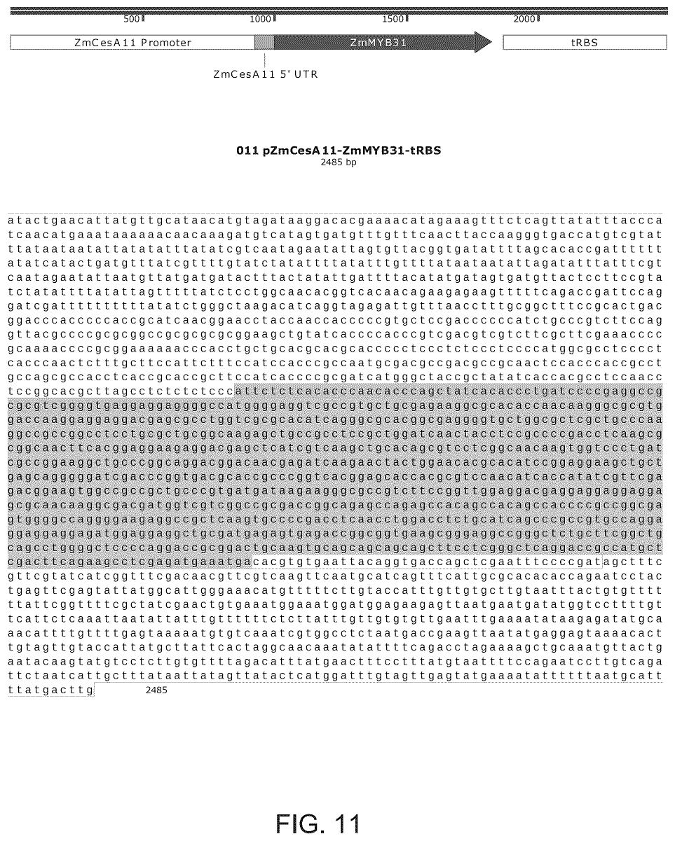

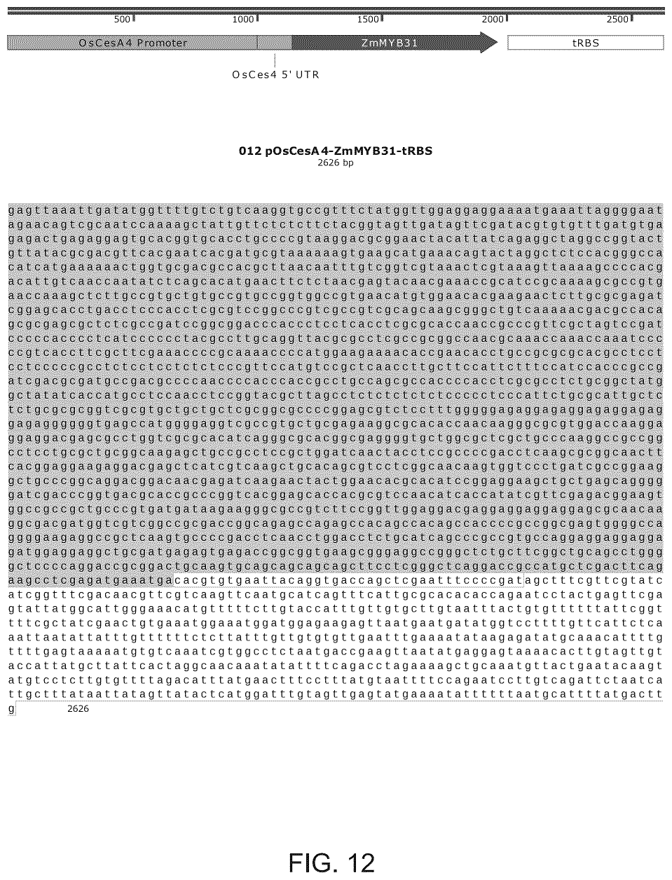

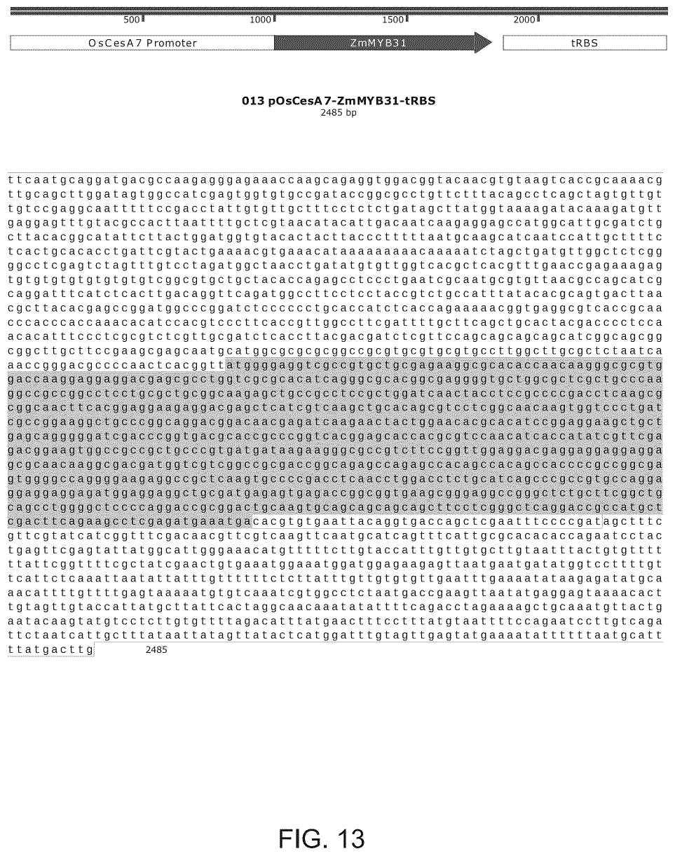

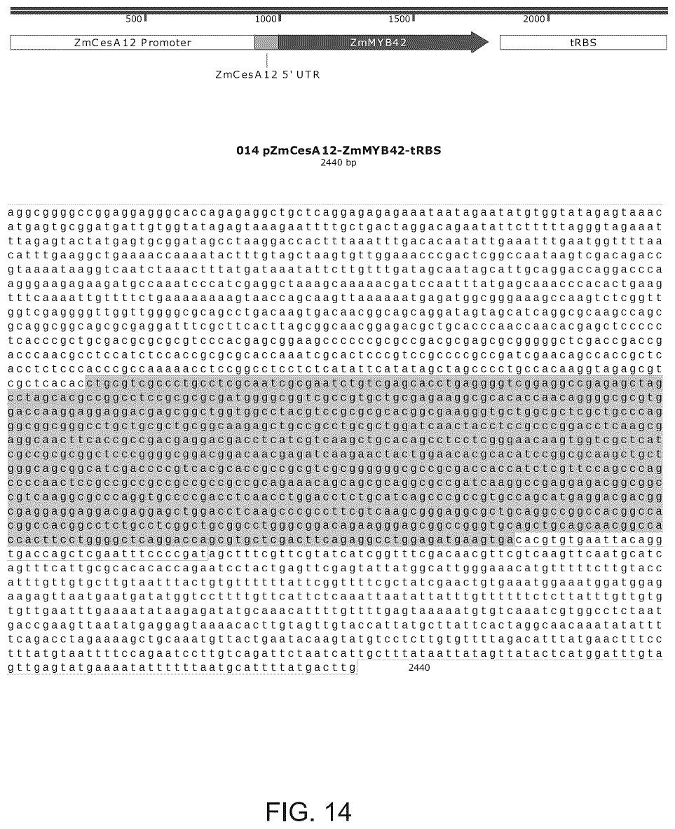

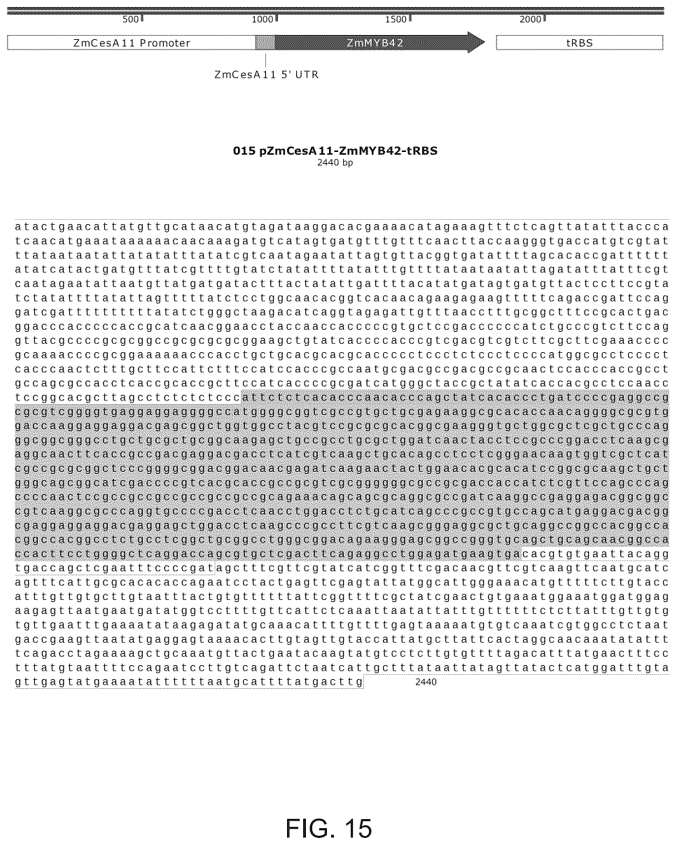

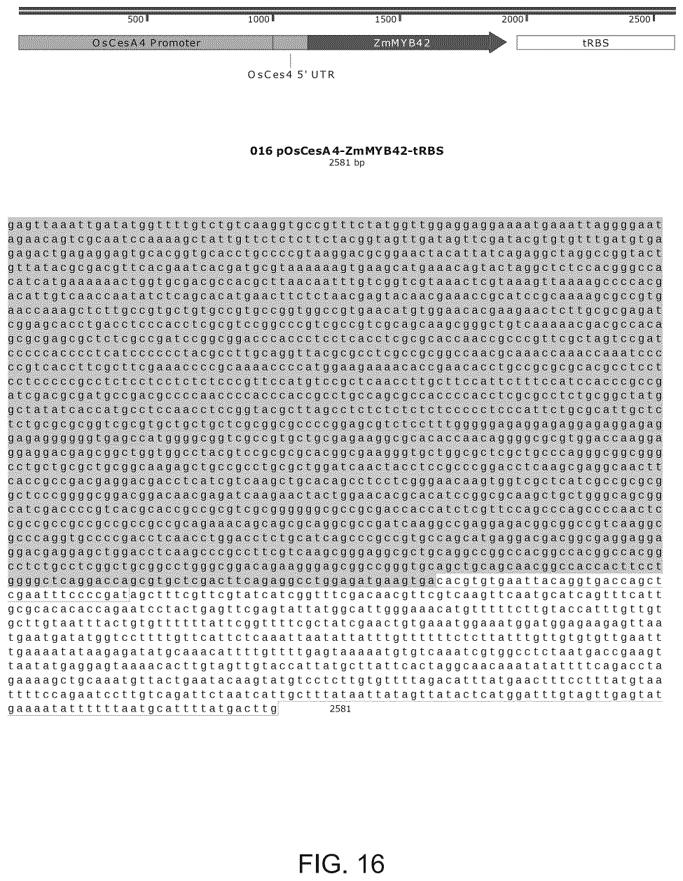

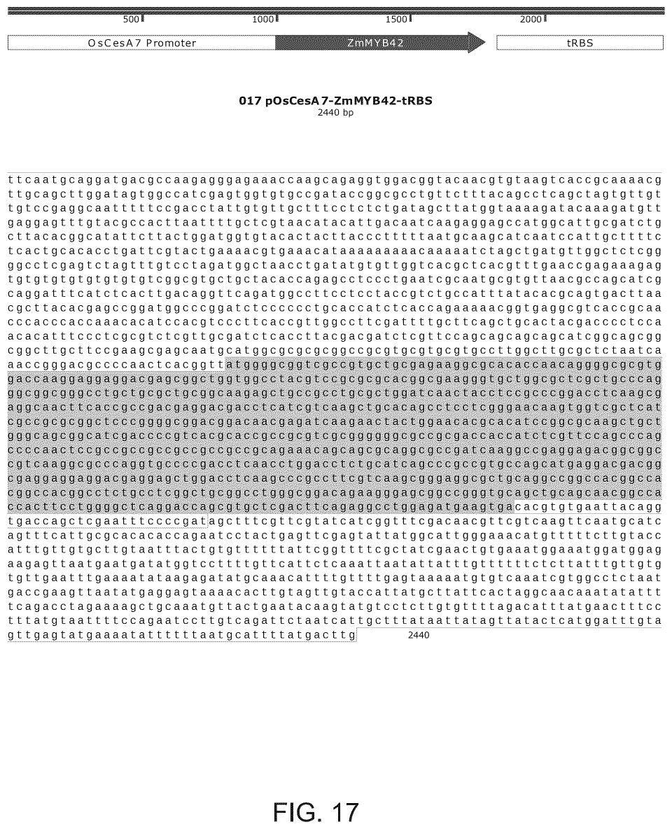

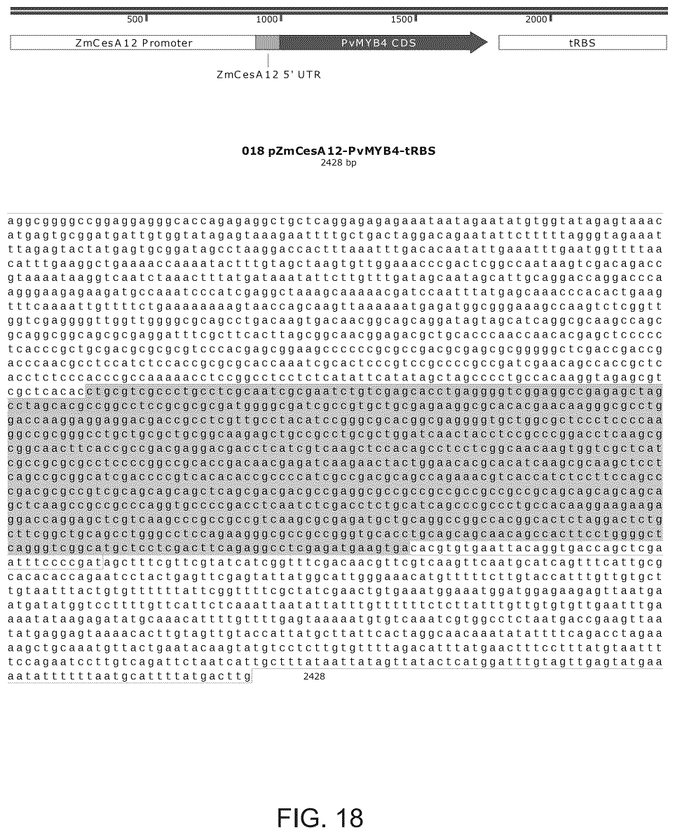

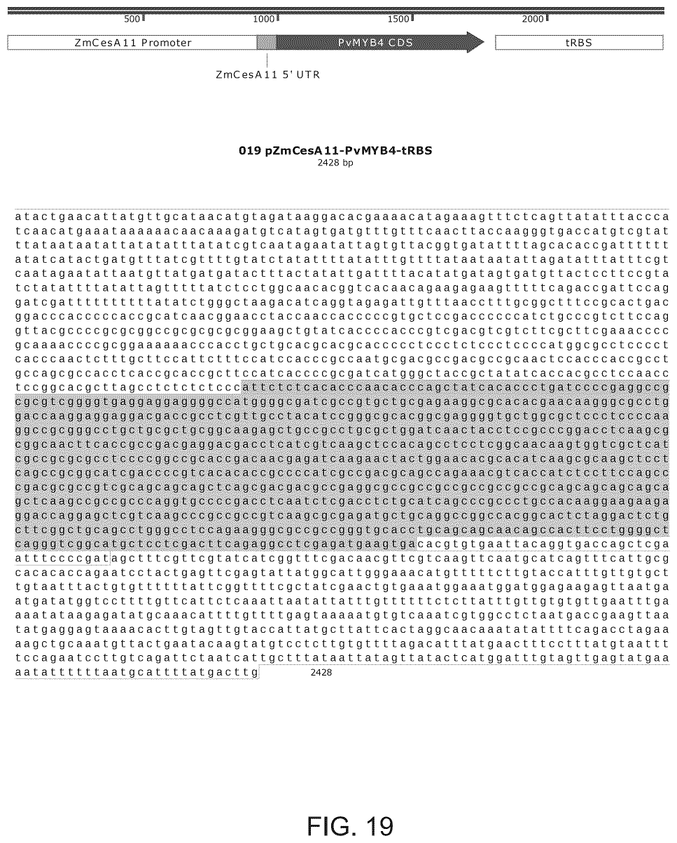

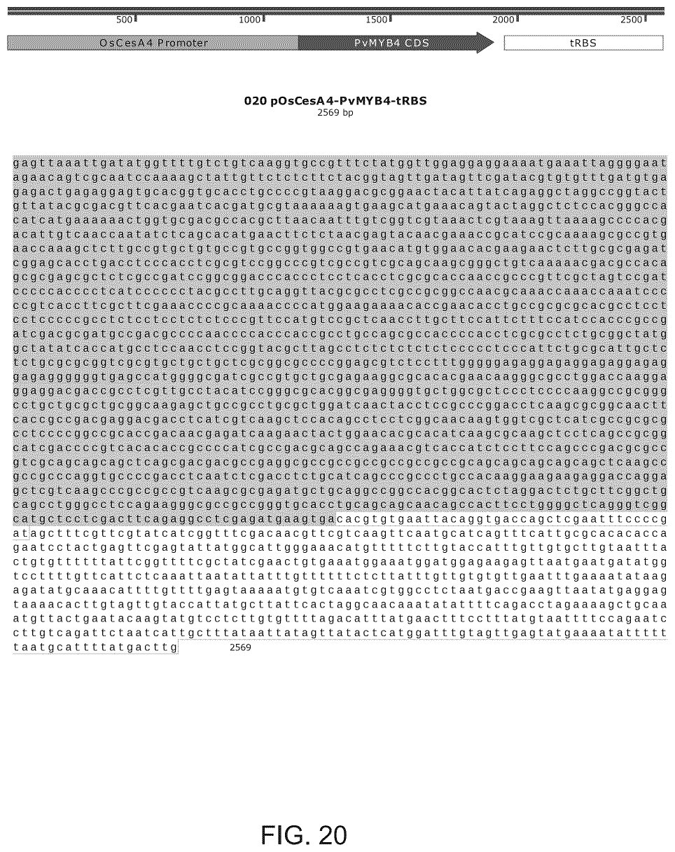

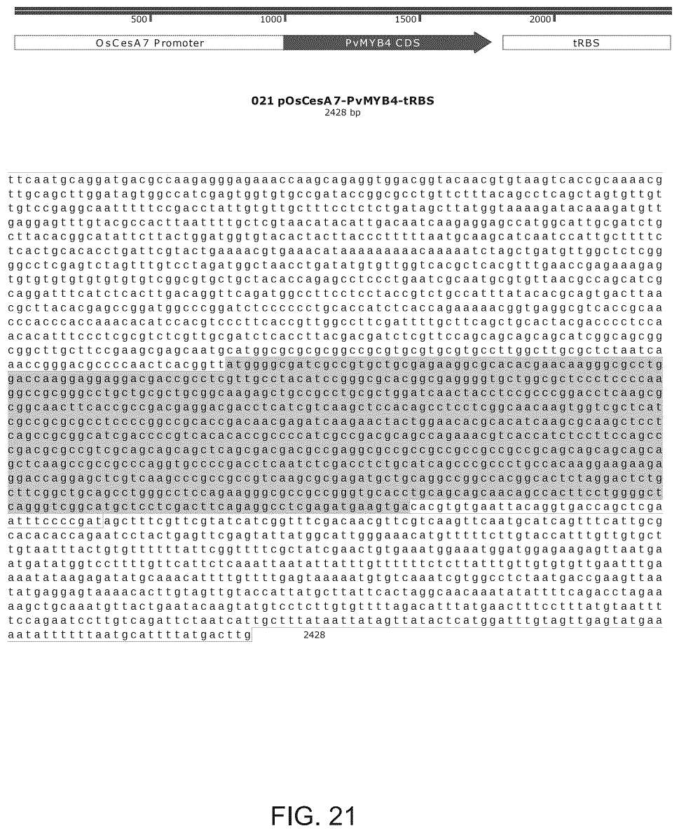

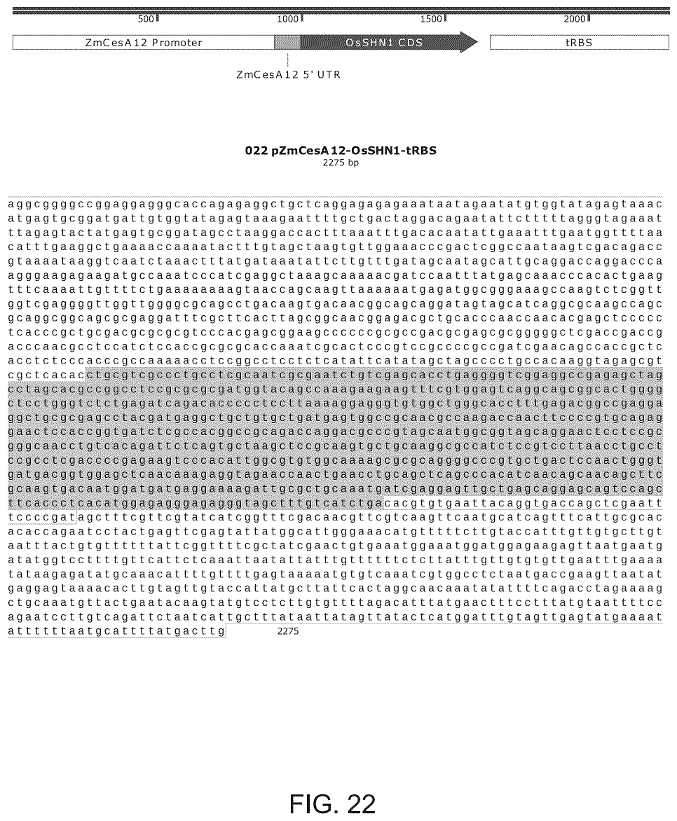

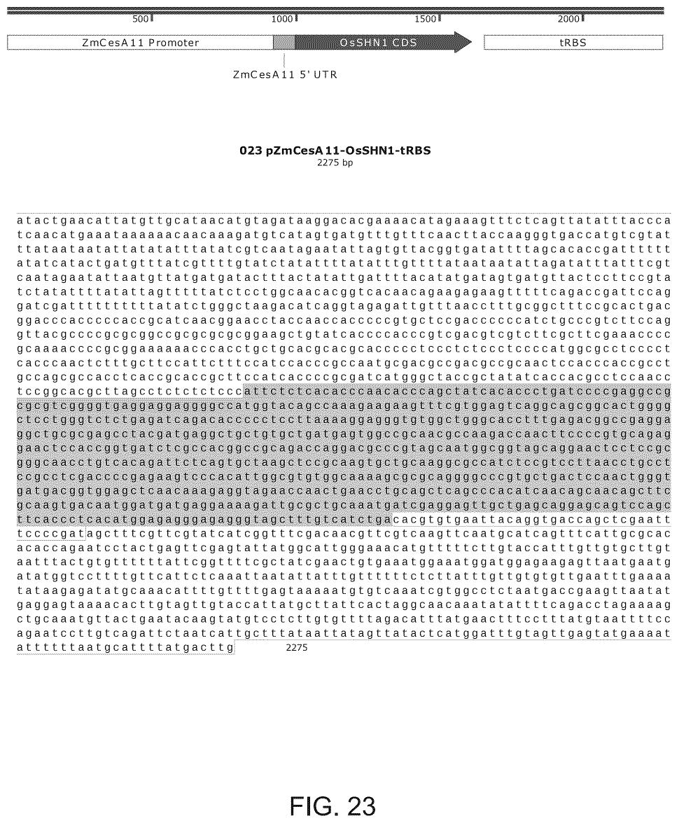

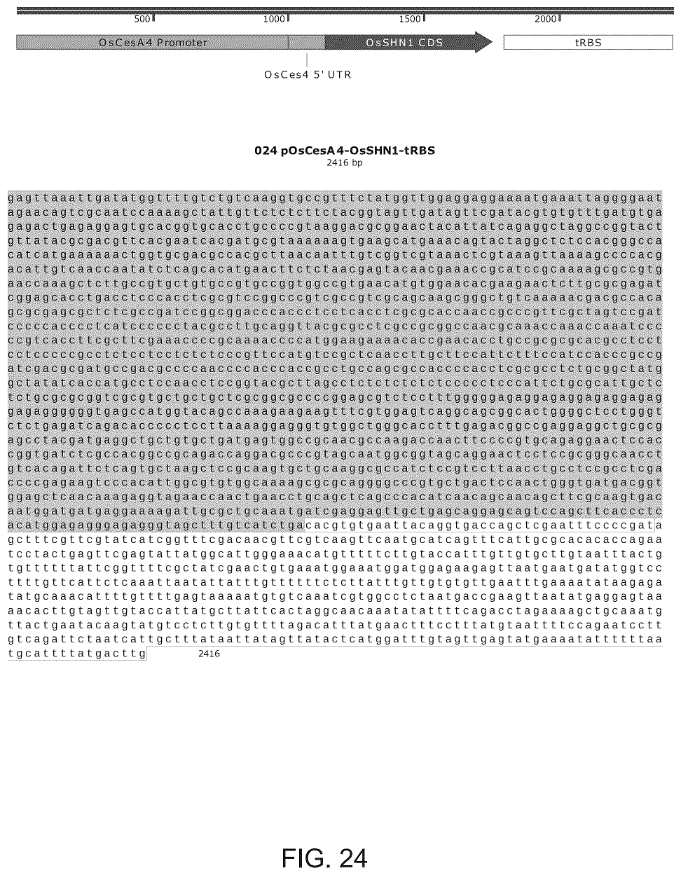

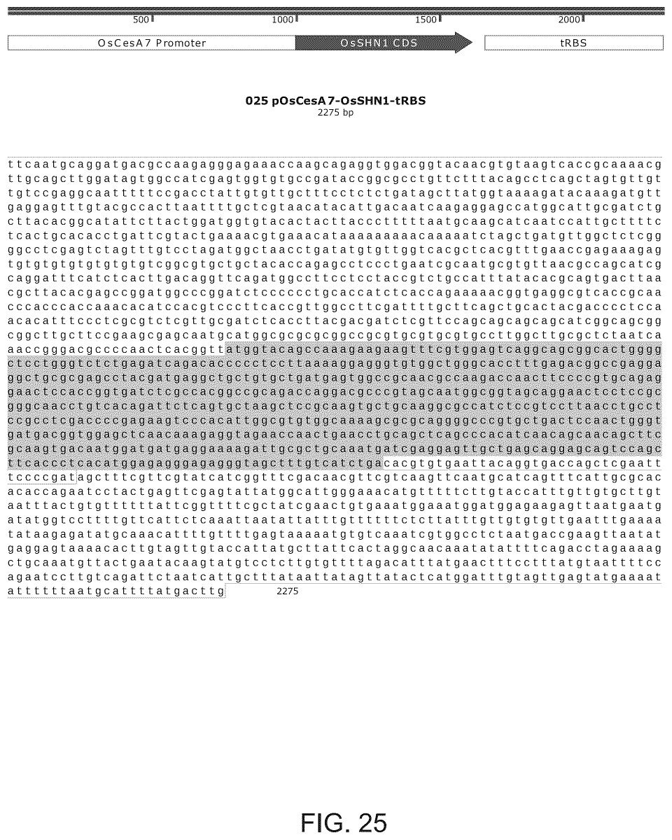

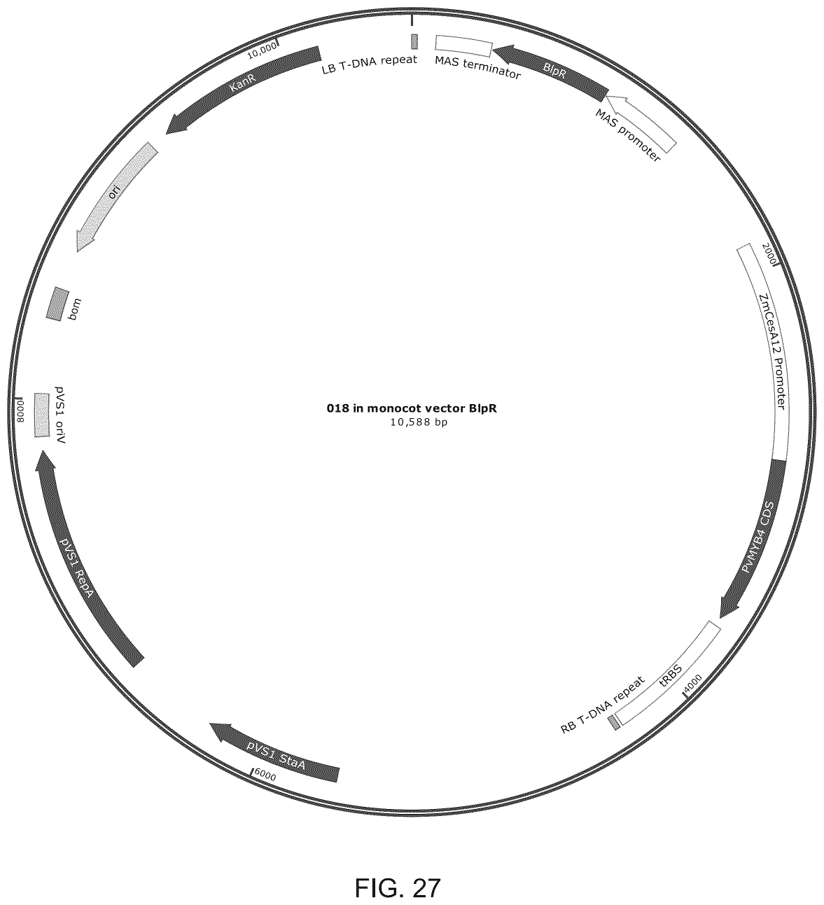

The two nucleotide sequences (promoter and coding regions) were then combined with RBS terminator region into a commonly used binary vector plasmid (including Kanamycin selection marker) through Gibson cloning (Gibson et al., “Enzymatic Assembly of DNA Molecules up to Several Hundred Kilobases,” Nature Methods 6(5):343-345 (2009), which is hereby incorporated by reference in its entirety). The result was assembly of constructs 010-025: pZmCesA12-ZmMYB31-tRBS (FIG. 10), pZmCesA11-ZmMYB31-tRBS (FIG. 11), pOsCesA4-ZmMYB31-tRBS (FIG. 12), pOsCesA7-ZmMYB31-tRBS (FIG. 13), pZmCesA12-ZmMYB42-tRBS (FIG. 14), pZmCesA11-ZmMYB42-tRBS (FIG. 15), pOsCesA4-ZmMYB42-tRBS (FIG. 16), pOsCesA7-ZmMYB42-tRBS (FIG. 17), pZmCesA12-PvMYB4-tRBS (FIG. 18), pZmCesA11-PvMYB4-tRBS (FIG. 19), pOsCesA4-PvMYB4-tRBS (FIG. 20), pOsCesA7-PvMYB4-tRBS (FIG. 21), pZmCesA12-OsSHN1-tRBS (FIG. 22), pZmCesA11-OsSHN1-tRBS (FIG. 23), pOsCesA4-OsSHN1-tRBS (FIG. 24), and pOsCesA7-OsSHN1-tRBS (FIG. 25). A representative plasmid map for construct 018 is shown in FIG. 27. Similar monocot-functional plasmids containing constructs 010-017 and 019-025 were also prepared.

Empty vectors corresponding to those shown in FIGS. 26 and 27, but lacking the constructs 001-025, were used as controls in the following molecular biology analysis and phenotypic analysis described in the following Examples.

### Example 2—Introduction of Construct Nos. 003 and 008 into Alfalfa (Medicago sativa L. Cv Regen S)

The vectors containing construct Nos. 003 and 008, along with the control vector, were used to generate transgenic alfalfa (Medicago sativa L. cv Regen S) using tissue culture and the Agrobacterium-mediated transformation method. After selection of primary transgenic plants using kanamycin, multiple events were obtained from the regeneration medium, cloned and propagated vegetatively, and transferred to soil after roots were developed. Introduction of the transgene cassette in the genome of the regenerated plants was confirmed by PCR using genomic DNA extracted from leaves.

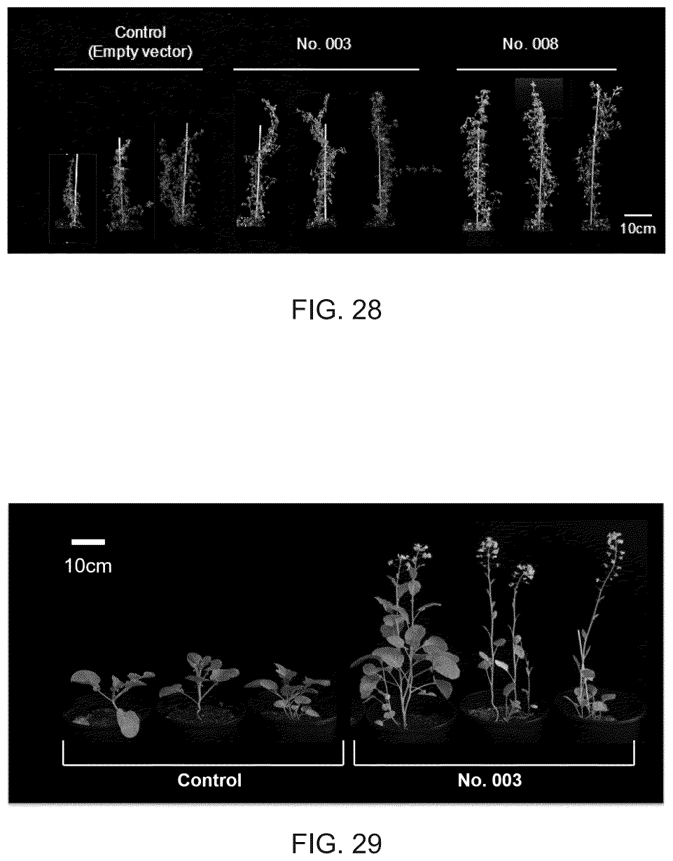

The experimentally confirmed and propagated plants were grown in individual pots inside growth chambers with 18/6-hour light/dark cycles. Table 4 shows biomass yield and stem internode length at approximately 250 days after propagation along with similar data for the control alfalfa plant with the construct. Engineered alfalfa shows approximately 15-18% longer internodes and 58-63% increased yields compared to the control plant. Representative images of control and inventive vector-transformed alfalfa plants are shown in FIG. 28.

TABLE 4
Biomass yield and stem length in alfalfa plants

| | Events | | Biomass yield (g) | |
|---|---|---|---|---|
| Construct | (n) | Stem length | Fresh weight | Dry weight |
| Control | 8 (17) | 57.56 ± 5.42 | 11.42 ± 2.99 | 2.20 ± 0.51 |
| No. 008 | 8 (21) | 66.47 ± 7.77*** | 17.74 ± 5.80*** | 3.49 ± 1.10*** |
| No. 003 | 7 (16) | 68.37 ± 6.28*** | 17.47 ± 3.95*** | 3.60 ± 0.73*** |

***p < 0.001

RNA was extracted from the leaves and stems in the engineered alfalfa lines for quantitative RT-PCR. Results show that the expression level of the target TF was similar to that of the native UbiQ gene. Moreover, transcript levels of the target TF were approximately 50 times higher in stem-enriched tissues compared to leaves, which highlights the tissue-preferential expression pattern enabled by the promoter used to drive expression of the TF. During the course of propagation, engineered alfalfa No. 003 also showed better development not only in stems but also significantly in roots. Table 5 shows regeneration efficiency and root length of alfalfa control and engineered No. 003 at 13 days after initiation of the regeneration step using sectioned internodes.

TABLE 5
Regeneration efficiency of new roots from sectioned stems

| | Prepared | Number | Rooting | Regenerated |
|---|---|---|---|---|
| | stem | of rooted | efficiency | root |
| Construct | fragments (n) | stems | (%) | length (mm) |
| Control | 59 | 10 | 16.95 | 1.91 ± 2.47 |
| | | | | (Max: 19.0) |
| No. 003 | 62 | 24 | 38.71 | 8.25** ± 5.90 |
| | | | | (Max: 29.0) |

**p < 0.01

The construct No. 003 and No. 008 alfalfa lines also showed a reduction of insoluble lignin and ash content (Table 6) and changes in lignin monomeric composition (Table 7), which indicate the inventive constructs also enhance biomass degradability through reduced lignin composition. Moreover, 12% and 16% less insoluble lignin content and 20% and 38% less ash content were observed in the No. 003 and No. 008 alfalfa lines, respectively, compared to control lines.

TABLE 6
Ash and insoluble lignin content in cell wall biomass

| Construct | Events (n) | Insoluble lignin (%) | Ash (%) |
|---|---|---|---|
| Control | 8 (8) | 12.96 ± 1.94 | 0.26 ± 0.09 |
| No. 008 | 11 (11) | 10.93* ± 0.86 | 0.16 ± 0.04 |
| No. 003 | 10 (10) | 11.45* ± 0.57 | 0.21* ± 0.04 |

*p < 0.05

TABLE 7
Lignin monomeric composition in cell wall biomass

| Construct | | | | |
|---|---|---|---|---|
| (events) | H unit (%) | G unit (%) | S unit (%) | S/G ratio |
| Control (3) | 1.77 ± 0.74 | 63.58 ± 1.24 | 34.65 ± 0.50 | 0.55 ± 0.02 |
| No. 008 (3) | 1.48 ± 0.34 | 48.39 ± 1.47 | 50.13 ± 1.76 | 1.04** ± 0.07 |
| No. 003 (3) | 1.48 ± 0.39 | 51.08 ± 3.28 | 46.99 ± 3.08 | 0.93* ± 0.11 |

**p < 0.01,
*p < 0.05

Table 8 summarizes the composition of saccharide released from cell wall fraction in the control lines and construct No. 003 and No. 008 lines. After a mild thermochemical treatment, glucose, xylose, and arabinose saccharides from constructs No. 003 and No. 008 biomass cell wall fraction were released two to three times more efficiently than the control lines.

TABLE 8
Composition of saccharide released from cell wall biomass

| Construct (events) | Glucose (%) | Xylose (%) | Arabinose (%) |
|---|---|---|---|
| Control (11) | 4.71 ± 2.46 | 8.01 ± 3.32 | 1.56 ± 0.18 |
| No. 008 (11) | 14.14** ± 3.79 | 15.78** ± 3.57 | 1.82** ± 0.18 |
| No. 003 (11) | 14.11** ± 4.05 | 15.84** ± 3.48 | 1.98*** ± 0.23 |

***p < 0.001,
**p < 0.01

### Example 3—Introduction of Construct No. 004 into Canola (Brassica napus L. Cv Westar)

The vector containing construct No. 004 and the control vector were used to generate transgenic canola (Brassica napus L. cv Westar) using tissue culture and the Agrobacterium-mediated transformation method. After selection of primary transgenic plants using kanamycin, multiple events were obtained on the regeneration agar plates and transferred to soil after vegetative tissue and roots were developed and cloned by vegetative propagation.

Table 9 summarizes the measured heights of five (5) transformed control canola lines compared to the four (4) No. 004 lines. The height of the plants transformed with the inventive method is approximately two times taller than the height of the control plants at 130 days after regeneration. These results indicate the inventive constructs enhance stem internode development and may increase biomass yield. Representative images of control and inventive vector-transformed canola plants are shown in FIG. 29.

TABLE 9
Plant height over 30-day period after regeneration

| | Events | Days after regeneration | | | |
|---|---|---|---|---|---|
| Construct | (n) | 100 | 110 | 120 | 130 |
| Control | 5 (5) | 18.58 ± 1.78 | 19.52 ± 1.82 | 20.38 ± 1.70 | 20.26 ± 1.75 |
| No. 004 | 4 (4) | 18.90 ± 0.70 | 22.35* ± 1.43 | 27.48** ± 3.23 | 46.45*** ± 4.18 |

***p < 0.001,
**p < 0.01,
*p < 0.05

The measured number of branches and flowers for five (5) control lines compared to the four (4) lines engineered with inventive constructs are shown in Table 10. Among the examined lines at day 150 after regeneration, two control lines and three No. 004 lines possessed grain pods, although grain in mature pods was observed in only No. 004 lines as shown in Table 11. All of the results indicate the inventive constructs enhance vegetative growth, root redevelopment as well as reproductive tissue development, and may increase overall yields.

TABLE 10
Numbers of Branch and Flower at
130 and 140 days after regeneration (DAR)

| | Events | Number of Branch | | Number of Flower | |
|---|---|---|---|---|---|
| Construct | (n) | 130 DAR | 140 DAR | 130 DAR | 140 DAR |
| Control | 5 (5) | 1.00 ± 0.00 | 1.40 ± 0.48 | 0.00 ± 0.00 | 0.00 ± 0.00 |
| No. 004 | 4 (4) | 4.00** ± 1.00 | 4.00* ± 1.00 | 12.00** ± 4.50 | 41.50*** ± 8.75 |

***p < 0.001,
**p < 0.01,
*p < 0.05

TABLE 11
Number of seed pods and seeds at 150 days after regeneration

| | Number of | Number | | Number |
|---|---|---|---|---|
| | plant with | of grain | Diameter of | of grain |
| Construct | grain pods | pods per event | pods (cm) | per pod |
| Control | 2 (2) | 5.50 ± 1.50 | 1.60 ± 0.10 | 0.00 ± 0.00 |
| No. 004 | 3 (3) | 25.67** ± 15.6 | 3.17** ± 1.38 | 6.67** ± 4.44 |

**p < 0.01

### Example 4—Introduction of Construct No. 018 into Sorghum (Sorghum bicolor P898012)

The vector containing construct No. 018 and the control vector were used to generate transgenic sorghum (Sorghum bicolor P898012) using tissue culture and the Agrobacterium-mediated transformation method. After selection of primary transgenic plants using glufosinate, multiple lines were obtained on the regeneration agar plates and transferred to soil after vegetative issue and roots were developed and cloned by vegetative propagation. Introduction of the transgene cassette was confirmed by PCR using genomic DNA extracted from the regenerated plant's leaves as the template.

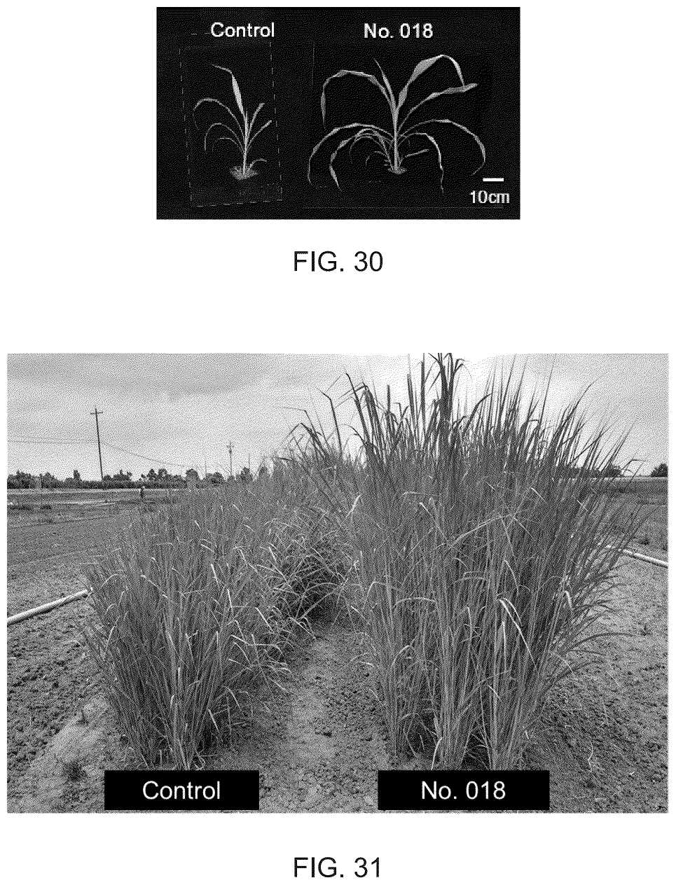

The experimentally confirmed and propagated plants were grown in individual pots inside the greenhouse with 18/6-hour light/dark cycles. Table 12 shows biomass yield, number of branches and plant height at approximately 250 days after propagation, along with similar data for sorghum control lines. The engineered sorghum lines showed approximately 80% more dry weight than the control lines, probably due to enhanced branching specific to the engineered lines. The obtained grain number and weight data, summarized in Table 13, indicate improved grain production. Representative images of control and inventive vector-transformed sorghum plants are shown in FIG. 30.

TABLE 12
Biomass yield and related-morphology data from engineered sorghum

| | Events | Plant height | Number of | Biomass yield |
|---|---|---|---|---|
| Construct | n) | (cm) | branches | (Dry weight: g) |
| Control | 8 (8) | 128.81 ± 15.98 | 0.00 ± 0.00 | 100.94 ± 25.14 |
| No. 018 | 8 (8) | 134.29 ± 16.41 | 6.57*** ± 1.63 | 183.29*** ± 14.17 |

***p < 0.001

TABLE 13
Grains from engineered sorghum

| | Events | Number of | Average grain |
|---|---|---|---|
| Construct | (n) | grains | weight (mg) |
| Control (8) | 8 (8) | 975 ± 84 | 40 |
| No. 018 (8) | 8 (8) | 4082* ± 526 | 40 |

*p < 0.05

### Example 5—Introduction of Construct No. 018 into Switchgrass (Panicum virgatum Alamo)

The vector containing construct No. 018 and the empty vector control were used to generate transgenic switchgrass (Panicum virgatum Alamo) using tissue culture and the Agrobacterium-mediated transformation method. After selection of primary transgenic plants using hygromycin, multiple events were obtained on the regeneration agar plates and transferred to soil after vegetative tissue and roots were developed and cloned by vegetative propagation. Introduction of the transgene cassette was confirmed by PCR using genomic DNA extracted from the regenerated plant's leaves as the template.

The experimentally confirmed and propagated plants were grown in individual pots with 18/6 hours light/dark cycles at a green house facility. RNA was extracted from the engineered switchgrass leaves and stems for quantitative RT-PCR. The analysis confirmed the target TF genes are mainly expressed in the stem rather than the leaves, suggesting that the tissue-preferred expression was enabled by the used cellulose synthase gene promoters.

During the course of transgenic plant generation, the engineered switchgrass with the gene cassette tended to show better differentiation and vegetative development. Table 14 compares plant height and number of tillers at 40 days after ratooning. In comparison to the control lines, approximately double the growth and 2-3× times more tillers were observed in the engineered lines. Representative images of control and inventive vector-transformed switchgrass plants are shown in FIG. 31.

TABLE 14
Plant height and number of tillers (40 days after ratooning)

| Construct | Event (n) | Plant height (cm) | Number of tillers |
|---|---|---|---|
| Control | 3 (3) | 83.00 ± 9.42 | 6.00 ± 2.83 |
| No. 010 | 5 (5) | 159.00** ± 18.14 | 22.80** ± 9.66 |
| No. 012 | 8 (8) | 178.00*** ± 10.09 | 32.88*** ± 8.33 |
| No. 013 | 5 (5) | 161.60*** ± 4.63 | 25.20** ± 10.72 |
| No. 020 | 7 (7) | 139.71** ± 23.14 | 24.00** ± 7.80 |
| No. 022 | 8 (8) | 164.13** ± 15.81 | 16.38** ± 4.47 |

***p < 0.001,
**p < 0.01

The switchgrass engineered by MYB TFs showed not only faster growth but also approximately 10-20% less insoluble lignin content (see Table 15: Constructs No. 010, No. 012, No. 013, and No. 020). Plants engineered by ERF TFs, however, maintained a similar amount of insoluble lignin (see Table 15: Construct No. 022), suggesting that the R2R3-MYB subfamily 4 and ERF/AP2 subfamily B-6 contribute to faster growth through distinguished mechanisms.

TABLE 15
Insoluble lignin content in upper and lower stem biomass

| | | Insoluble lignin content (%) | |
|---|---|---|---|
| Construct | Event (n) | Upper stem biomass | Lower stem biomass |
| WT | 6 | 17.46 ± 0.59 | 21.13 ± 0.46 |
| Control | 3 (11) | 17.06 ± 0.41 | 20.00 ± 0.90 |
| No. 010 | 5 (5) | 15.92** ± 0.32 | 18.62** ± 0.54 |
| No. 012 | 5 (5) | 15.95** ± 0.92 | 18.33** ± 0.90 |
| No. 013 | 4 (4) | 15.15** ± 0.25 | 17.44** ± 0.34 |
| No. 020 | 4 (4) | 15.51** ± 0.41 | 16.96** ± 0.16 |
| No. 022 | 7 (7) | 17.59 ± 1.47 | 21.18 ± 0.85 |

**p < 0.01

Faster development in the reproductive phase was also observed in the engineered lines. Mature seeds produced after the completion of reproductive development were harvested and quantitatively analyzed (Table 16). Among engineered lines, construct No. 012, No. 013, and No. 018 switchgrass produced large quantities of seeds, approximately 6-7× more seeds than the control lines.

TABLE 16
Number of mature seeds produced

| Construct | Event (n) | Number of seeds produced |
|---|---|---|
| Control | 11 (11) | 165.4 ± 39.9 |
| No. 012 | 12 (12) | 915.3** ± 153.7 |
| No. 013 | 5 (5) | 1012.0** ± 153.2 |
| No. 018 | 7 (7) | 1109.0* ± 451.4 |

**p < 0.01,
*p < 0.05

Germinating efficiency from the obtained T1 seeds was examined by using a 96 well format system. Two containers that included pre-soaked sponges with 96 well halls were prepared for seed planting and used for the gemination process in dark conditions at 25° C. Time-course observation of the germinated seedlings confirmed that the seeds from construct No. 012 and No. 018 switchgrass enhances not only the gemination rate but also seedling development (seedling length) in comparison to the control lines (Table 17).

TABLE 17
Time course of T1 seedling length (cm)

| | Days after seed planting | | | | | | |
|---|---|---|---|---|---|---|---|
| Construct | 0 | 8 | 10 | 13 | 16 | 20 | 23 |
| Control | 0.00 ± | 0.19 ± | 0.49 ± | 1.35 ± | 1.42 ± | 1.75 ± | 3.31 ± |
| Container 1 | 0.00 | 0.48 | 0.84 | 1.14 | 1.17 | 1.20 | 1.78 |
| Control | 0.00 ± | 0.00 ± | 0.29 ± | 1.13 ± | 1.45 ± | 2.06 ± | 3.76 ± |
| Container 2 | 0.00 | 0.00 | 0.51 | 1.32 | 1.37 | 1.50 | 2.49 |
| No. 012 | 0.00 ± | 2.22** ± | 3.57** ± | 4.40** ± | 4.61** ± | 4.92** ± | 5.48** ± |
| Container 1 | 0.00 | 1.92 | 2.36 | 2.61 | 2.70 | 2.56 | 2.43 |
| No. 012 | 0.00 ± | 2.22** ± | 3.25** ± | 4.03** ± | 4.19** ± | 4.78** ± | 5.74** ± |
| Container 2 | 0.00 | 1.57 | 1.85 | 1.34 | 1.22 | 0.99 | 0.96 |
| No. 018 | 0.00 ± | 1.77** ± | 2.20** ± | 3.10** ± | 3.80** ± | 4.77** ± | 6.35** ± |
| Container 1 | 0.00 | 1.72 | 2.32 | 2.35 | 2.52 | 2.48 | 2.94 |
| No. 018 | 0.00 ± | 2.07** ± | 2.61** ± | 3.37** ± | 4.16** ± | 5.06** ± | 6.50** ± |
| Container 2 | 0.00 | 1.97 | 2.59 | 2.63 | 2.82 | 2.67 | 2.54 |

**p < 0.01

Growth and morphology of the engineered lines were also examined in a field environment. Switchgrass plantlets were grown under greenhouse conditions and transplanted to field plots with a total of 1,000 square feet. A total of 30 plantlets was distributed to each plot, and a total of 120 plantlets were planted per construct. Table 18 shows biomass data for constructs No. 012 and No. 018 switchgrass that yielded approximately 35% more than wild-type and control lines.

TABLE 18
Biomass yield (total dry weight) from the field test

| | | Number | Biomass yield (kg) |
|---|---|---|---|
| Construct | n per plot | of plots | from one plot |
| WT | 30 | 4 | 8.82 ± 1.04 |
| Control | 30 | 4 | 8.44 ± 1.09 |
| No. 012 | 30 | 4 | 12.14** ± 1.29 |
| No. 018 | 30 | 4 | 12.96** ± 1.21 |

**p < 0.01

Construct No. 012 and No. 018 plants grown in field conditions also showed cell wall characteristics similar to those observed in laboratory-grown plants. As shown in Table 19, the constructs had 10% less insoluble lignin content and higher S/G unit composition ratios in comparison to wild-type switchgrass, suggesting the engineered switchgrass could be useful as potential forage with better digestibility and/or as less recalcitrant feedstock for a cost-effective biorefinery process.

TABLE 19
Lignin characteristics from the field test.

| | Insoluble | | | |
|---|---|---|---|---|
| Construct | lignin (%) | G unit (%) | S (%) | S/G ratio |
| WT | 22.74 ± 1.94 | 75.01 ± 0.05 | 24.99 ± 0.47 | 0.33 ± 0.0008 |
| No. 012 | 20.48** ± 1.58 | 69.47** ± 0.70 | 30.53** ± 0.53 | 0.44** ± 0.01 |
| No. 018 | 20.90** ± 0.78 | 65.49** ± 0.53 | 34.51** ± 0.54 | 0.53** ± 0.01 |

**p < 0.01

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method of producing a transgenic plant having improved traits as compared to a non-transgenic or wild-type control plant of the same plant species, the method comprises:
a. introducing into a plant cell a nucleic acid construct consisting of a heterologous plant tissue-specific promoter which is operably linked to a polynucleotide encoding a polypeptide of subgroup 4 R2R3-MYB transcription factor that shares at least 60% sequence similarity with SEQ ID NO: 04, and obtaining transformed plant cells;
b. obtaining transgenic plants expressing the polypeptide from the transformed plant cells of step a); and
c. selecting a transgenic plant from the transgenic plants of step b) that expresses the polypeptide, and wherein expression of the polypeptide in the selected transgenic plant exhibits both increased biomass yield and lower insoluble lignin content, as compared to the control plant.

2. The method according to claim 1, wherein the polypeptide is SEQ ID NO: 04, 05, 19, or 20.

3. The method according to claim 1, wherein the polypeptide is SEQ ID NO: 04, and wherein the selected transgenic plant further exhibits at least one of i) increased plant height; ii) increased number of tillers/branches; iii) enhanced root development; iv) increased seed production, v) increased ratio of syringyl to guaiacyl monomeric units in lignin; and vi) increased saccharide release from plant cell wall, as compared to the control plant.

4. The method according to claim 3, wherein the tissue-specific promoter is SEQ ID NO: 23 or 24.

5. The method according to claim 1, wherein the polypeptide is SEQ ID NO: 05, and wherein the selected transgenic plant further exhibits at least one of i) increased plant height; ii) increased number of tillers/branches; iii) increased ratio of syringyl to guaiacyl monomeric units in lignin; and iv) increased saccharide release from plant cell wall, as compared to the control plant.

6. The method according to claim 5, wherein the tissue-specific promoter is SEQ ID NO: 23.

7. The method according to claim 1, wherein the polypeptide is SEQ ID NO: 19, and wherein the selected transgenic plant further exhibits at least one of i) increased plant height; ii) increased number of tillers/branches; iii) increased seed production; iv) enhanced germination and seedling development; and v) increased ratio of syringyl to guaiacyl monomeric units in lignin, as compared to the control plant.

8. The method according to claim 7, wherein the tissue-specific promoter is SEQ ID NO: 35, 30, or 31.

9. The method according to claim 1, wherein the polypeptide is SEQ ID NO: 20, and wherein the selected transgenic plant further exhibits at least one of i) increased plant height; ii) increased number of tillers/branches; iii) increased seed production; iv) enhanced germination and seedling development; and v) increased ratio of syringyl to guaiacyl monomeric units in lignin, as compared to the control plant.

10. The method according to claim 9, wherein the tissue-specific promoter is SEQ ID NO: 35 or 30.

11. The method according to claim 1, wherein the nucleic acid construct further comprises a 3′ transcription termination site, wherein the 3′ transcription termination site is operably linked to the polynucleotide.

12. The method according to claim 1, wherein the nucleic acid construct is present on a plasmid capable of being transformed into a plant.

13. The method according to claim 1, wherein the nucleic acid construct is present in Agrobacterium for plant transformation.

14. The method according to claim 1, wherein the nucleic acid construct is introduced into the plant cell by infective transformation, electroporation, direct uptake, microinjection, or biolistic transformation.

15. The method according to claim 1, wherein the nucleic acid construct is introduced into the plant cell by transfection.

16. The method according to claim 1, wherein the nucleic acid construct is introduced into the plant cell by transfection into a bacterium for plant transformation.
